(19) United States
(12) Patent Application Publication
(10) Pub. No.: US 2025/0283016 A1
(43) Pub. Date: Sep. 11, 2025

(54) Polypeptides Having Beta-Glucanase Activity, Polynucleotides Encoding Same and Uses Thereof In Cleaning and Detergent Compositions

(71) Applicant: Novozymes A/S, Bagsvaerd (DK)

(72) Inventors: Iben DAMAGER, Vaerloese (DK); Morten GJERMANSEN, Greve (DK)

(73) Assignee: Novozymes A/S, Bagsvaerd (DK)

(21) Appl. No.: 19/220,781

(22) Filed: May 28, 2025

Related U.S. Application Data
(62) Division of application No. 17/601,027, filed on Oct. 1, 2021.

(30) Foreign Application Priority Data
Apr. 3, 2019 (EP) 19167059.5
Jan. 31, 2020 (EP) 20154957.3

(51) Int. Cl. C11D 3/386; C12N 9/20; C12N 9/22; C12N 9/24; C12N 15/75

(52) U.S. Cl. CPC C11D 3/38636; C11D 3/38618; C11D 3/38627; C11D 3/38645; C12N 9/20; C12N 9/22; C12N 9/2402; C12N 9/2405; C12N 9/2488; C12N 15/75; C12Y 302/01001; C12Y 302/01004; C12Y 302/01006; C12Y 302/01015; C11D 2111/12; C11D 2111/14

(57) ABSTRACT

The invention relates to cleaning or detergent compositions comprising polypeptides exhibiting beta-glucanase activity, optionally comprising one or more amylases and/or one or more proteases and uses thereof in cleaning or detergent applications and processes such as cleaning hard-surfaces, dish wash and laundering. The present invention relates to polypeptides having beta-glucanase activity, catalytic domains, beta-glucan binding domains and polynucleotides encoding the polypeptides, catalytic domains or beta-glucan binding domains. The invention also relates to nucleic acid constructs, vectors, and host cells comprising the polynucleotides as well as methods of producing and using the polypeptides, catalytic domains or beta-glucan binding domains.

### CROSS-REFERENCE TO RELATED APPLICATIONS

This application is a divisional of U.S. application Ser. No. 17/601,027 filed on Oct. 1, 2021, now pending, which is a 35 U.S.C. 371 national application of international application no. PCT/EP2020/059359 filed Apr. 2, 2020 and published on Oct. 8, 2020 as WO 2020/201403, which claims priority or the benefit under 35 U.S.C. 119 of European application no. 19167059.5 filed Apr. 3, 2019 and European application no. 20154957.3 filed Jan. 31, 2020. The disclosure of each application is fully incorporated herein by reference.

### REFERENCE TO A SEQUENCE LISTING

This application contains a Sequence Listing in computer readable form, which was created on May 27, 2025, is named SQ.xml and is 130,458 bytes in size. The Sequence Listing is hereby incorporated by reference in its entirety.

### BACKGROUND OF THE INVENTION

### Field of the Invention

The present invention relates to cleaning or detergent compositions comprising polypeptides exhibiting beta-glucanase activity and one or more amylases and/or one or more proteases and uses thereof in cleaning or detergent applications and processes such as cleaning hard-surfaces, dish wash and laundering. The present invention further relates to polypeptides having beta-glucanase activity and polynucleotides encoding the polypeptides. The invention also relates to nucleic acid constructs, vectors, and host cells comprising the polynucleotides as well as methods of producing and using the polypeptides, e.g., in cleaning or detergent applications and processes such as cleaning hard-surfaces, dish wash and laundering.

### Description of the Related Art

Beta-glucans are polysaccharides consisting of glucose units linked by beta-glycosidic bonds. Cellulose is one type of beta-glucan, in which all of the glucose units are linked by beta-1,4-glucosidic bonds. This feature results in the formation of insoluble cellulose micro-fibrils. Enzymatic hydrolysis of cellulose to glucose requires the use of endo beta-glucanases (e.g., EC 3.2.1.4), cellobiohydrolases (e.g., EC 3.2.1.91) and beta-glucosidases (e.g., EC 3.2.1.21).

Beta-glucans can also be linked by beta-1,3-glucosidic bonds (e.g., as found in the cell walls of baker's yeast, Saccharomyces cerevisiae), beta-1,6-glucosidic bonds as well as combinations of beta-1,3-, beta-1,4- and beta-1,6-glucosidic bonds. The combination of beta-1,3- and beta-1,4-glucosidic bonds can be found, e.g., in the soluble fibre from cereals such as oats and barley. In addition, storage polysaccharides found in algae contain 1,3-linked beta-D-glucose residues with various degrees of 1,6-branching. A subgroup of beta-glucanases, also known as laminarinases, can be classified as endo-1,3-beta-glucanases (EC 3.2.1.6 and EC 3.2.1.39) or exo-1,3-beta-glucanases (EC 3.2.1.58). Laminarinases can be used to catalyse the hydrolysis of the beta-1,3-glucosidic bonds, or beta-1,4-glucosidic bonds when the glucose residue whose reducing group is involved in the linkage to be hydrolysed is substituted at C3 to release glucose or oligosaccharides. These enzymes can act on laminarin, lichenin and cereal beta-D-glucans, but not on substrates containing only 1,4-bonds.

Other beta-glucanases (e.g., EC 3.2.1.4) can, for example, perform endohydrolysis of (1,4)-beta-D-glucosidic linkages in cellulose, lichenin and cereal beta-D-glucans and will also hydrolyze 1,4-linkages in beta-D-glucans containing 1,3-linkages. Still other beta-glucanases (e.g., licheninases EC 3.2.1.73)) can hydrolyze (1,4)-beta-D-glucosidic linkages in beta-D-glucans containing (1,3)- and (1,4)-bonds, but not on substrates containing only 1,3- or only 1,4-bonds.

The removal of cereal stains as oat and barley containing stains in dish wash and laundry is a recognised problem, and there is a considerable interest in finding enzymes that can degrade the beta-glucans found therein.

The present invention provides polypeptides of glycoside hydrolyase family 16 (GH16) having beta-glucanase activity (e.g., comprising or consisting of laminarinase (EC 3.2.1.6, EC 3.2.1.39, and/or EC 3.2.1.58) activity) and polynucleotides encoding said polypeptides, which are highly active in degrading different types of beta-glucans (e.g., linear or branched beta-1,3-glucans), and therefore could be used in the aforementioned applications, e.g., in cleaning or detergent applications and processes such as cleaning hard-surfaces, dish wash and laundering. The existing products comprising beta-glucanases have very low effect on this type of beta-glucan as their main enzymatic substrate is cellulose. Therefore, the present invention provides novel beta-glucanases with improved properties (e.g., with significant improvement of performance and/or stability under alkaline conditions; and optionally, beta-glucanases without cellulase activity (e.g., not having endo-cellulase activity on β-1,4 linkages between D-glucose units) (e.g., EC 3.2.1.6, EC 3.2.1.39, and/or EC 3.2.1.58 activity). A difference between use of cellulases and laminarinases on textile in laundry is that the laminarinases do not degrade the fibers of the textile.

Furthermore, some particular solid detergents have pH above 10. The known beta-glucanases are not suitable for these very high pH detergents. The present invention provides novel beta-glucanases with improved properties (e.g., with significant improvement of performance and/or stability under alkaline conditions).

An uncharacterized protein from Paenibacillus sp. SMB1 (TREMBL:A0A2W1L111) is 79.1% identical to the beta-glucanase shown in SEQ ID NO: 3.

An uncharacterized protein from Paenibacillus sp. FSL A5-0031 (SWISSPROT:A0A1R0ZTD2) is 98.3% identical to the beta-glucanase shown in SEQ ID NO: 6.

A protein from Cohnella sp. A01 (SWISSPROT:A0A173DRP6) is 85.6% identical to the beta-glucanase shown in SEQ ID NO: 9.

A protein from Paenibacillus elgii(TREMBL:A0A2T6FW69) is 97.3% identical to the beta-glucanase shown in SEQ ID NO: 12.

A protein from Bacillus sp. C1-1 (TREMBL:A0A3N9Q4J6) is 89.4% identical to the beta-glucanase shown in SEQ ID NO: 15.

A protein from Bacillus patagoniensis 065DS (AHGP:EFP7Q40PC) is 92.5% identical to the beta-glucanase shown in SEQ ID NO: 18.

### SUMMARY OF THE INVENTION

The invention relates to cleaning composition comprising a polypeptide having beta-glucanase activity, wherein the polypeptide is a gram-positive bacteria of order Bacillales and comprises a motif selected from the group consisting of NXAXGG (SEQ ID NO: 30), GEXDXME (SEQ ID NO: 28), GXGNXEXXXY (SEQ ID NO: 29), YTS[G/A][K/R](SEQ ID NO: 31) and combinations thereof, and at least one cleaning component, preferably selected from a surfactant, a builder, a bleach component, a polymer, a dispersing agent and/or an additional enzyme.

In one aspect, the composition comprises a polypeptide, wherein said beta-glucanase activity is laminarinase activity EC 3.2.1.6, EC 3.2.1.39, or EC 3.2.1.58, preferably EC 3.2.1.6. In one aspect, the composition comprises a polypeptide, wherein the polypeptide has endo-1,3-beta-glucanase activity, e.g., EC 3.2.1.6 or EC 3.2.1.39.

In one aspect, the composition comprises a polypeptide, wherein the polypeptide is obtained from a strain of Bacillus, e.g., Bacillus sp., a strain of Paenibacillus, e.g., Paenibacillus elgii or Paenibacillus sp., a strain of Thermobacillus, e.g., Thermobacillus sp. or from a strain of Cohnella, e.g., Cohnella sp.

In one aspect, the composition comprises a polypeptide, which comprises a motif selected from the group consisting of [L/M]NXAXGG, LNXAXGG (SEQ ID NO: 43), GEIDIME (SEQ ID NO: 32), G[F/W]GNXEX[Q/E]XY (SEQ ID NO: 33), and combinations thereof. In one embodiment, the composition comprises a polypeptide which comprises each of the motifs LNXAXGG (SEQ ID NO: 43), GXGNXEXXXY (SEQ ID NO: 29), and GEXDXME (SEQ ID NO: 28).

In one aspect, the composition comprises a polypeptide which comprises, consists, or consists essentially of a polypeptide having an amino acid sequence selected from the group consisting of SEQ ID NO: 12, SEQ ID NO: 3, SEQ ID NO: 6, SEQ ID NO: 9, SEQ ID NO: 15, and SEQ ID NO: 18, or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto.

In one aspect the composition is a cleaning composition such as a laundry or dish wash composition.

In one aspect, the composition of comprises (i) one or more polypeptides having amylase activity, such as alpha-amylase activity; and/or (ii) one or more polypeptides having protease activity.

In one aspect, the composition comprises one or more additional enzymes, such as cellulases, DNases, lipases, mannanases, pectinases, as well as combinations of these, also optionally combined with amylases or proteases.

The invention also relates to polypeptide of a gram-positive bacteria of order Bacillales and comprises a motif selected from the group consisting of NXAXGG (SEQ ID NO: 30), GEXDXME (SEQ ID NO: 28), GXGNXEXXXY (SEQ ID NO: 29), YTS[G/A][K/R](SEQ ID NO: 31) and combinations thereof.

In one aspect, the polypeptide has laminarinase activity EC 3.2.1.6, EC 3.2.1.39, or EC 3.2.1.58, preferably EC 3.2.1.6. In one aspect, the polypeptide has endo-1,3-beta-glucanase activity, e.g., EC 3.2.1.6 or EC 3.2.1.39.

In one aspect, the polypeptide is obtained from a strain of Bacillus, e.g., Bacillus sp., a strain of Paenibacillus, e.g., Paenibacillus elgii or Paenibacillus sp., a strain of Thermobacillus, e.g., Thermobacillus sp. or from a strain of Cohnella, e.g., Cohnella sp.

In one aspect, the polypeptide comprises a motif selected from the group consisting of [L/M]NXAXGG, LNXAXGG (SEQ ID NO: 43), GEIDIME (SEQ ID NO: 32), G[F/W]GNXEX[Q/E]XY (SEQ ID NO: 33), and combinations thereof. In one embodiment, the polypeptide comprises each of the motifs LNXAXGG (SEQ ID NO: 43), GXGNXEXXXY (SEQ ID NO: 29), and GEXDXME (SEQ ID NO: 28).

In one aspect, the polypeptide comprises, consists, or consists essentially of a polypeptide having an amino acid sequence selected from the group consisting of SEQ ID NO: 12, SEQ ID NO: 3, SEQ ID NO: 6, SEQ ID NO: 9, SEQ ID NO: 15, and SEQ ID NO: 18, or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto.

One aspect of the invention relates to a polynucleotide encoding a polypeptide of the invention. The invention further relates to nucleic acid construct or expression vector comprising the polynucleotide. The invention further relates to a host cell comprising a polypeptide of the invention.

One aspect relates to the use of a polypeptide of the invention for reducing or preventing soil redeposition; removal of cereal containing soil, especially dried-on cereal containing soil, preferably oat flakes containing soil, especially dried-on oat flakes containing soil and/or cooked oats containing soil, and/or cooked and burned-in oats containing soil, and/or uncooked oats containing soil; removal of chocolate containing soil, especially chocolate porridge oats containing soil, and/or chocolate milkshake containing soil, and/or chocolate drinks containing soil; removal of cosmetics and/or personal care containing soil; removal of tomato containing soil, especially tomato soup containing soil, and/or tomato sauce, such as spaghetti sauce containing soil; facilitating removal of starch-containing soil in the presence of one or more amylases and/or for enhancing amylase related cleaning performance; facilitating removal of protein-containing soil in the presence of one or more proteases and/or for enhancing protease related cleaning performance; facilitating removal of carbohydrase-containing soil in the presence of one or more other carbohydrases and/or enhancing carbohydrase related cleaning performance; reducing or removing a biofilm from an item, such as textile, preferably in a cleaning process such as laundry; and/or cleaning, e.g., deep cleaning of an item, wherein the item is a textile or a surface.

One aspect relates to a method of producing the polypeptide of the invention, comprising: (a) cultivating the recombinant host cell under conditions conducive for production of the polypeptide; and (b) recovering the polypeptide.

The invention further relates to a cleaning or laundering method for cleaning or laundering an item comprising the steps of (a) exposing an item to a wash liquor comprising a polypeptide of the invention or a detergent composition comprising the polypeptide of the invention; (b) completing at least one wash cycle; and optionally rinsing the item.

### Overview of Sequence Listing

SEQ ID NO: 1 is the DNA sequence of the beta-glucanase as isolated from a strain of a Thermobacillus sp.

SEQ ID NO: 2 is the amino acid sequence of the beta-glucanase as deduced from SEQ ID NO: 1.

SEQ ID NO: 3 mature polypeptide obtained from Thermobacillus sp.

SEQ ID NO: 4 is the DNA sequence of the beta-glucanase as isolated from a strain of a Paenibacillus sp.

SEQ ID NO: 5 is the amino acid sequence of the beta-glucanase as deduced from SEQ ID NO: 4.

SEQ ID NO: 6 mature polypeptide obtained from Paenibacillus sp.

SEQ ID NO: 7 is the DNA sequence of the beta-glucanase as isolated from a strain of a Cohnella sp. SEQ ID NO: 8 is the amino acid sequence of the beta-glucanase as deduced from SEQ ID NO: 7.

SEQ ID NO: 9 mature polypeptide obtained from Cohnella sp.

SEQ ID NO: 10 is the DNA sequence of the beta-glucanase as isolated from a strain of a Paenibacillus elgii.

SEQ ID NO: 11 is the amino acid sequence of the beta-glucanase as deduced from SEQ ID NO: 10.

SEQ ID NO: 12 mature polypeptide obtained from Paenibacillus elgii.

SEQ ID NO: 13 is the DNA sequence of the beta-glucanase as isolated from a strain of a Bacillus species A.

SEQ ID NO: 14 is the amino acid sequence of the beta-glucanase as deduced from SEQ ID NO: 13.

SEQ ID NO: 15 mature polypeptide obtained from Bacillus species A.

SEQ ID NO: 16 is the DNA sequence of the beta-glucanase as isolated from a strain of a Bacillus species B.

SEQ ID NO: 17 is the amino acid sequence of the beta-glucanase as deduced from SEQ ID NO: 16.

SEQ ID NO: 18 mature polypeptide obtained from Bacillus species B.

SEQ ID NO: 19 is a polypeptide secretion signal Bacillus clausii.

SEQ ID NO: 20 is an artificial N-terminal poly-histidine affinity purification tag sequence.

SEQ ID NO: 21 is His-tagged recombinant mature beta-glucanase protein from Thermobacillus sp.

SEQ ID NO: 22 is His-tagged recombinant mature beta-glucanase protein from Paenibacillus sp.

SEQ ID NO: 23 is His-tagged recombinant mature beta-glucanase protein from Cohnella sp.

SEQ ID NO: 24 is His-tagged recombinant mature beta-glucanase protein from Paenibacillus elgii.

SEQ ID NO: 25 is His-tagged recombinant mature beta-glucanase protein from Bacillus species A.

SEQ ID NO: 26 is His-tagged recombinant mature beta-glucanase protein from Bacillus species B.

SEQ ID NO: 27 is beta-glucanase from Thermotoga maritima.

motif
SEQ ID NO: 28
GEXDXME
motif
SEQ ID NO: 29
GXGNXEXXXY
motif
SEQ ID NO: 30
NXAXGG
motif
SEQ ID NO: 31
YTS[G/A][K/R]
motif
SEQ ID NO: 32
GEIDIME
motif
SEQ ID NO: 33
G[F/W]GNXEX[Q/E]XY
motif
SEQ ID NO: 34
GFGNXEXQXY
motif
SEQ ID NO: 35
GFGNXEXEXY
motif
SEQ ID NO: 36
GWGNXEXQXY
motif
SEQ ID NO: 37
GWGNXEXEXY
motif
SEQ ID NO: 38
YTSGK
motif
SEQ ID NO: 39
YTSGR
motif
SEQ ID NO: 40
YTSAK
motif
SEQ ID NO: 41
YTSAR
motif
SEQ ID NO: 42
[L/M]NXAXGG
motif
SEQ ID NO: 43
LNXAXGG
motif
SEQ ID NO: 44
MNXAXGG

SEQ ID NO: 45 is an artificial amylase protein sequence.

SEQ ID NO: 46 is an amylase protein sequence from Bacillus sp.

SEQ ID NO: 47 is an amylase protein sequence from Bacillus sp.

SEQ ID NO: 48 is a polypeptide corresponding to SEQ ID NO: 2 of WO 95/10603.

SEQ ID NO: 49 is a polypeptide corresponding to SEQ ID NO: 6 in WO 02/010355.

SEQ ID NO: 50 is a polypeptide corresponding to a hybrid polypeptide comprising residues 1-33 of SEQ ID NO: 6 of WO 2006/066594 and residues 36-483 of SEQ ID NO: 4 of WO 2006/066594.

SEQ ID NO: 51 is a polypeptide corresponding to SEQ ID NO: 6 of WO 02/019467.

SEQ ID NO: 52, SEQ ID NO: 53 and SEQ ID NO: 54 are polypeptides respectively corresponding to SEQ ID NO: 1, SEQ ID NO: 2 or SEQ ID NO: 7 of WO 96/23873.

SEQ ID NO: 55 is a polypeptide corresponding to SEQ ID NO: 2 of WO 08/153815 SEQ ID NO: 56 is a polypeptide corresponding to SEQ ID NO: 10 of WO 01/66712.

SEQ ID NO: 57 is a polypeptide corresponding to SEQ ID NO: 2 of WO 09/061380.

SEQ ID NO: 58 is an amylase protein sequence from Bacillus sp.

SEQ ID NO: 59 is an amylase protein sequence from Bacillus sp.

SEQ ID NO: 60 is an amylase protein sequence from Bacillus sp.

SEQ ID NO: 61 is an amylase protein sequence from Cytophaga sp.

SEQ ID NO: 62 is an amylase protein sequence from Bacillus sp.

SEQ ID NO:63 is an amylase protein sequence from Bacillus sp.

SEQ ID NO: 64 is an amylase protein sequence from Bacillus halmapalus.

SEQ ID NO: 65 is an artificial amylase protein sequence.

SEQ ID NO: 66 is an amylase protein sequence from Bacillus sp.

SEQ ID NO: 67 is a protease protein sequence from Bacillus lentus.

SEQ ID NO: 68 is an artificial protease protein sequence.

SEQ ID NO: 69 is an artificial protease protein sequence.

SEQ ID NO: 70 is an artificial protease protein sequence.

SEQ ID NO: 71 is a cellulase from Bacillus sp.

SEQ ID NO: 72 is a cellulase from Humicola insolens

SEQ ID NO: 73 is a cellulase from Humicola insolens

SEQ ID NO: 74 is a cellulase from Thielavia terrestris

SEQ ID NO: 75 is a cellulase from Paenibacillus polymyxa

SEQ ID NO: 76 is a cellulase from Melanocarpus albomyces

SEQ ID NO: 77 is a lipase from Thermomyces lanuginosus

SEQ ID NO: 78 is a mannanase from Bacillus bogoriensis

SEQ ID NO: 79 is a mannanase from Paenibacillus sp.

SEQ ID NO: 80 is a mannanase from Bacillus hemicellulosilyticus

SEQ ID NO: 81 is a mannanase from Paenibacillus woosongensis

SEQ ID NO: 82 is a mannanase from Paenibacillus woosongensis

SEQ ID NO: 83 is a mannanase from Paenibacillus illinoisensis

SEQ ID NO: 84 is a mannanase from Paenibacillus illinoisensis

SEQ ID NO: 85 is a mannanase from Neobulgaria sp.

SEQ ID NO: 86 is a mannanase from Preussia aemulans

SEQ ID NO: 87 is a mannanase from Yunnania penicillate

SEQ ID NO: 88 is a mannanase from Myrothecium roridum

SEQ ID NO: 89 is a mannanase from Chaetomium brasiliense

SEQ ID NO: 90 is a mannanase from Ascobolus stictoideus

SEQ ID NO: 91 is a mannanase from Chaetomium virescens

SEQ ID NO: 92 is a pectinase from Bacillus subtilis

SEQ ID NO: 93 is a DNase from Bacillus cibi

SEQ ID NO: 94 is a DNase from Aspergillus oryzae

### Definitions

Anti-redeposition: The term “anti-redeposition” or “anti-redeposition effect” means the reduction or prevention of soil from depositing back onto the textile, fabric or hard surface. The anti-redeposition effect can be determined using the Mini-LOM or Mini-TOM wash assay as described in the examples herein (e.g., as in example 14).

Synergistic effect: The term “synergistic effect” means a cooperative action of polypeptides such that a total combined effect of said polypeptides is greater than the sum of the individual enzymatic effects of said polypeptides. Non-limiting examples of synergistic effect include REM synergistic effect of a beta-glucanase polypeptide of the invention and one or more alpha-amylase (and/or one or more proteases).

REM synergistic effect: REM synergistic effect of polypeptides as used herein can be measured based on the analysis of stain removal carried out by using any suitable wash performance methodology (e.g., Wascator bottle wash method). A preferred method for determining the REM synergistic effect is disclosed in Examples disclosed herein, e.g., Example 7.

Beta-glucanase: The term “beta-glucanase” as used herein means an endo-acting enzyme that catalyzes the hydrolysis of a beta-1,3-, beta-1,6- and/or beta-1,4-bonds connecting two glucosyl residues in a beta-glucan. Non-limiting examples of beta-glucanases as defined herein include cellulases (e.g., EC 3.2.1.4, e.g., having endo-cellulase activity on β-1,4 linkages between D-glucose units and laminarinases (e.g., EC 3.2.1.6, EC 3.2.1.39, and/or EC 3.2.1.58), as described below. For purposes of the present invention, beta-glucanase activity is determined according to the procedure described in the Examples. In one aspect of the invention, the polypeptides of the present invention have at least 20%, e.g., at least 40%, at least 50%, at least 60%, at least 70%, at least 80%, at least 90%, at least 95%, or at least 100% of the beta-glucanase activity of the polypeptide having the sequence selected from the group consisting of: SEQ ID NO: 3, SEQ ID NO: 6, SEQ ID NO: 9, SEQ ID NO: 12, SEQ ID NO: 15, SEQ ID NO: 18. Beta-glucanase activity can suitably be measured using beta-glucan as substrate. A preferred assay for determining beta-glucanase activity is disclosed in Example 1 (AZCL-curdlan or pachyman beta-glucan assay). A further subgroup of beta-glucanases as defined herein, also known as a laminarinases (e.g., EC 3.2.1.6, EC 3.2.1.39, and/or EC 3.2.1.58), can also be used to catalyse the hydrolysis of the beta-1,3-glucosidic bonds, or beta-1,4-glucosidic bonds when the glucose residue whose reducing group is involved in the linkage to be hydrolysed is substituted at C3 to release glucose or oligosaccharides. As used herein the term “beta-glucanase activity” comprises laminarinase (e.g., EC 3.2.1.6, EC 3.2.1.39, and/or EC 3.2.1.58) activity.

Beta-glucan: The term “beta-glucan” as used herein means a polysaccharide that only contain glucose as structural components, and in which the glucose units are linked by beta-glycosidic bonds. Non-limiting examples of beta-glucans include beta-D-glucans, beta-1,3-1,4-glucans, mix-linkage beta-glucans, barley beta-glucans, oatmeal beta-glucans, beta-1,3-glucans and beta-1,3-1,6-glucans.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Amylase: The term “amylase” (EC 3.2.1) refers to enzymes which catalyze the hydrolysis of starch, glycogen, and related polysaccharides to oligosaccharides, maltose, or glucose. Amylases are glycoside hydrolases and act on α-1,4-glycosidic bonds. The amylases suitable in the cleaning compositions of the invention are preferably alpha amylases. Alpha-amylases (EC 3.2.1.1) includes 1,4-α-D-glucan glucanohydrolase and glycogenase and are calcium metalloenzymes. By acting at random locations along the starch chain, alpha-amylase breaks down long-chain carbohydrates, ultimately yielding maltotriose and maltose from amylose, or maltose, glucose and “limit dextrin” from amylopectin. Suitable amylases of the present invention are preferably microbial, e.g., obtained from bacterial or fungal sources. The term “alpha-amylase activity” means the activity of alpha 1,4-glucan 4 glucanohydrolases, E.C. 3.2.1.1, which constitute a group of enzymes, which catalyze hydrolysis of starch and other linear and branched 1,4 alpha-glucosidic oligo and poly-saccharides. Alpha-amylase activity may be determined by Assay II as described in the Examples herein

Biofilm: The term “biofilm” means any group of microorganisms in which cells stick to each other on a surface, such as a textile, dishware or hard surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from free-floating bacteria of the same species, as the dense and protected environment of the film allows them to cooperate and interact in various ways. One effect of this environment is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

Carbohydrate binding module: The term “carbohydrate binding module” means the region within a carbohydrate-active enzyme that provides carbohydrate-binding activity (Boraston et al., 2004, Biochem. J. 383: 769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. Some CBMs are known to have specificity for cellulose.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellulolytic enzyme or cellulase: The term “cellulolytic enzyme” or “cellulase” means one or more (e.g., several) enzymes that hydrolyze a cellulosic material. Such enzymes include endoglucanase(s) (e.g., EC 3.2.1.4), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman No. 1 filter paper as the substrate. The assay was established by the International Union of Pure and Applied Chemistry (IUPAC) (Ghose, 1987, Pure Appl. Chem. 59: 257-68). Cellulase activity may be determined by Assay III as described in the Examples herein.

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Deep cleaning: By the term “deep cleaning” is meant reduction, disruption or removal of components, which may be comprised in organic matter, e.g., skin debris, dead cell material, sebum, sweat and biofilm, such as polysaccharides, grease, proteins, starch, DNA, soil or other components present in the organic matter. The organic matter may be termed poly-organic stains comprising more than one organic component such as starch, grease, protein, DNA and mannan.

Detergent component: the term “detergent component” is defined herein to mean the types of chemicals which can be used in detergent compositions. Examples of detergent components are surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

Detergent composition: the term “detergent composition” refers to compositions that find use in the removal of undesired compounds from items to be cleaned, such as textiles, dishes, and hard surfaces. The detergent composition may be used to, e.g., clean textiles, dishes and hard surfaces for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, plastic, ceramic and metal counter tops and windows; carpet cleaners; oven cleaners; fabric fresheners; fabric softeners; and textile and laundry pre-spotters, as well as dish wash detergents). In addition to containing a GH16 beta-glucanase of the invention, the detergent formulation may contain one or more additional enzymes (such as amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases (GH5 and/or GH26), licheninases, phosphodiesterases, pectin methylesterases, cellobiohydrolases, transglutaminases, and combinations thereof, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g., by hand dish wash (HDW) or automatic dish wash (ADW). Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present invention is not restricted to any particular type of dish wash composition or any particular detergent.

DNases: DNases are polypeptides with DNase (deoxyribonuclease) activity that catalyzes the hydrolytic cleavage of phosphodiester linkages in a DNA backbone, thus degrading DNA. Exodeoxyribonuclease cut or cleaves residues at the end of the DNA back bone where endo-deoxyribonucleases cleaves or cut within the DNA backbone. A DNase may cleave only double-stranded DNA or may cleave double stranded and single stranded DNA. The term “DNases” and the expression “a polypeptide with DNase activity” may be used interchangeably throughout the application. For purposes of the present invention, DNase activity may determined according to the procedure described in the Assay IV or Assay V of the Examples herein. Preferably the DNase is selected from any of the enzyme classes E.C.3.1, preferably E.C.3.1.21. Preferably, the polypeptide having DNase activity is obtained from a microorganism and the DNase is a microbial enzyme. The DNase is preferably of fungal or of bacterial origin.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic or carbohydrate binding module having one or more (e.g., several) amino acids absent from the amino and/or carboxyl terminus of a mature polypeptide or domain; wherein the fragment has beta-glucanase or carbohydrate binding activity. In one aspect, a fragment contains at least 456 amino acid residues, or at least 432 amino acid residues, or at least 408 amino acid residues, wherein the fragment has beta-glucanase activity (e.g., amino acids 1 to 408, amino acids 1 to 432, amino acids 1 to 456 of SEQ ID NO: 3). In one aspect, a fragment contains at least 248 amino acids, or at least 235 amino acid residues, or at least 222 amino acid residues (e.g., amino acids 1 to 222, amino acids 1 to 235, amino acids 1 to 248 of SEQ ID NO: 6). In one aspect, a fragment contains at least 365 amino acid residues, or at least 346 amino acid residues, or at least 327 amino acid residues (e.g., amino acids 1 to 327, amino acids 1 to 346, amino acids 1 to 365 of SEQ ID NO: 9). In one aspect, a fragment contains at least 362 amino acid residues, or at least 343 amino acid residues, or at least 324 amino acid residues (e.g., amino acids 1 to 324, amino acids 1 to 344, amino acids 1 to 362 of SEQ ID NO: 12). In one aspect, a fragment contains at least 247 amino acids, at least 234 amino acids, at least 221 amino acids (e.g., amino acids 1 to 221, amino acids 1 to 234, amino acids 1 to 247 of SEQ ID NO: 15). In one aspect, a fragment contains at least 243 amino acids, at least 230 amino acids, at least 217 amino acids (e.g., amino acids 1 to 217, amino acids 1 to 230, amino acids 1 to 247 of SEQ ID NO: 18).

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a GH5 mannanase, a GH26 mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication, as well as a recombinant host cell, an isolated host cell (e.g., an isolated recombinant host cell), an isolated host cell that is not a human embryonic stem cell. In preferred embodiments of the invention a recombinant host cell is a heterologous recombinant host cell (e.g., a host cell that is not a Bacillus agaradhaerens host cell, or a host cell that is not a Bacillus sp-62449 host cell, or a host cell that is not a Bacillus akibai host cell, or a host cell that is not a Bacillus mojavensis host cell).

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). A fermentation broth produced by culturing a recombinant host cell expressing the polynucleotide of the invention will comprise the polypeptide of the invention in an isolated form.

Laundering: The term “laundering” relates to both household laundering and industrial laundering and means the process of treating textiles with a solution containing a cleaning or detergent composition of the present invention. The laundering process can for example be carried out using, e.g., a household or an industrial washing machine or can be carried out by hand.

Laminarinase activity: The term “laminarinase activity” means enzymes that hydrolyse beta-1,3-glucans (e.g., EC 3.2.1.6, EC 3.2.1.39, and/or EC 3.2.1.58).

As used herein, the classification EC 3.2.1.6 is synonymous with endo-1,3(4)-beta-glucanase, and includes those laminarinases that catalyze the endo-hydrolysis of 1,3- or 1,4-linkages in β-D-glucans, when the glucose residue whose reducing group is involved in the linkage to be hydrolyzed is itself substituted at C-3 and the classification EC 3.2.1.39 as used herein is synonymous with glucan endo-1,3-beta-D-glucosidase, and includes laminarinases that hydrolyse the (1-3)-beta-D-glucosidic linkages in (1-3)-beta-D-glucans. EC 3.2.1.39 is different from EC 3.2.1.6 in showing very limited action on mixed-link (1-3, 1-4)-beta-D-glucans. Laminarinase activity may be determined according to Assay I in the Examples.

Lipase: The term lipase includes enzymes which catalyze the hydrolysis of fats (lipids). Lipases are a sub class of esterases. Lipases suitable in the present invention include phospholipases, acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase in particular the S54V variant used in the commercial product Gentle Power Bleach from Huntsman Textile Effects Pte Ltd (WO 2010/100028). Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147). Lipase activity may be determined as described in Assay VI in the Examples herein.

Mannanase: The term “mannanase” includes enzymes that catalyzes the hydrolysis of mannans, which is a highly branched polymer of mannose. The mannanases of the invention are preferably of microbial origin such as bacterial or fungal mannanases. The mannanase preferably having mannan endo-1,4-beta-mannosidase activity (EC 3.2.1.78) that catalyzes the hydrolysis of 1,4-3-D-mannosidic linkages in mannans, galactomannans and/or glucomannans. The mannanase may be a GH5 mannanase such as an endo-1,4-β-Mannanase or a GH26 endo-1,4 β-Mannanase. Mannanase activity may be determined as described in Assay VII in the Examples herein.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is selected from the group consisting of: amino acids 1 to 480 of SEQ ID NO: 2, amino acids 1 to 480 of SEQ ID NO: 3, amino acids 1 to 262 of SEQ ID NO: 5, amino acids 1 to 262 of SEQ ID NO: 6, amino acids 1 to 385 of SEQ ID NO: 8, amino acids 1 to 385 of SEQ ID NO: 9, amino acids 1 to 382 of SEQ ID NO: 11, amino acids 1 to 382 of SEQ ID NO: 12, amino acids 1 to 260 of SEQ ID NO: 14, amino acids 1 to 260 of SEQ ID NO: 15, amino acids 1 to 256 of SEQ ID NO: 17, amino acids 1 to 256 of SEQ ID NO: 18. The amino acids −36 to −1 of SEQ ID NO: 2 are a signal peptide. The amino acids −28 to −1 of SEQ ID NO: 5 are a signal peptide. The amino acids −31 to −1 of SEQ ID NO: 8 are a signal peptide. The amino acids −28 to −1 of SEQ ID NO: 11 are a signal peptide. The amino acids −24 to −1 of SEQ ID NO: 14 are a signal peptide. The amino acids −23 to −1 of SEQ ID NO: 17 are a signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having beta-glucanase activity. In one aspect, the mature polypeptide coding sequence is selected from the group consisting of: nucleotides 109 to 1548 of SEQ ID NO: 1, nucleotides 854 to 870 of SEQ ID NO: 4, nucleotides 94 to 1248 of SEQ ID NO: 7, nucleotides 85 to 1230 of SEQ ID NO: 10, nucleotides 73 to 852 of SEQ ID NO: 13, nucleotides 70 to 837 of SEQ ID NO: 16. The nucleotides 1 to 108 of SEQ ID NO: 1 encode a signal peptide. The nucleotides 1 to 84 of SEQ ID NO: 4 encode a signal peptide. The nucleotides 1 to 93 of SEQ ID NO: 7 encode a signal peptide. The nucleotides 1 to 84 of SEQ ID NO: 108 encode a signal peptide. The nucleotides 1 to 72 of SEQ ID NO: 13 encode a signal peptide. The nucleotides 1 to 69 of SEQ ID NO: 16 encode a signal peptide.

Malodor: The term “malodor” means an odor which is not desired on clean items. The cleaned item should smell fresh and clean without malodors adhered to the item. One example of malodor is compounds with an unpleasant smell, which may be produced by microorganisms. Another example is sweat or body odor adhered to an item which has been in contact with humans or animals. Another example of malodor can be the smell from spices, for example curry or other exotic spices adhering to an item such as a piece of textile. One way of measuring the ability of an item to adhere malodor is by using the Malodor Assay.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Pectinase: The term “pectinase” denotes a pectinase enzyme defined according to the art and includes an enzyme that cleaves poly- and/or oligosaccharide chains in pectic substances, e.g., poly(1,4-alpha-D-galacturonide) and its derivatives (see reference Sakai et al., Pectin, pectinase and protopectinase: production, properties and applications, pp 213-294 in: Advances in Applied Microbiology vol:39, 1993). Non-limiting examples of pectinases include hydrolase type pectinases (e.g., rhamnogalacturonan hydrolases) and lyase type pectinases (e.g., pectate lyases). Preferably a pectinase of the invention is a pectinase enzyme which catalyzes the random cleavage of alpha-1,4-glycosidic linkages in pectic acid also called polygalacturonic acid by transelimination such as the enzyme class polygalacturonate lyase (EC 4.2.2.2) (PGL) also known as poly(1,4-alpha-D-galacturonide) lyase also known as pectate lyase. Pectinase activity may be determined as described in Assay VIII in the Examples herein.

Protease: The term “protease” includes enzymes that hydrolyze peptide bonds and the term incudes peptidase and proteinase. Serine proteases (or serine endopeptidases), E.C. 3.4.21 are enzymes that cleave peptide bonds in proteins, in which serine serves as the nucleophilic amino acid at the active site. Suitable proteases include those of bacterial, fungal, plant, viral or animal origin, e.g., vegetable or microbial origin. Microbial origin is preferred. Chemically modified or protein engineered mutants are included. Most relevant proteases for laundry may be the alkaline proteases, such as a serine protease. A serine protease may for example be of the S1 family, such as trypsin, or the S8 family such as subtilisin. A metalloproteases protease may for example be a thermolysin from, e.g., family M4 or other metalloproteases such as those from M5, M7 or M8 families. The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e., the Subtilisin family, the Thermitase family, the Proteinase K family, the Lantibiotic peptidase family, the Kexin family and the Pyrolysin family. Protease activity may be determined as described in Assay IX in the Examples herein.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

$$( Identical Deoxyribonucleotides \times 100 ) / ( Length of Alignment - Total Number of Gaps in Alignment )$$

Stringency conditions: The different stringency conditions are defined as follows.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 1.6×SSC, 0.2% SDS at 60° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.8×SSC, 0.2% SDS at 60° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.8×SSC, 0.2% SDS at 65° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.4×SSC, 0.2% SDS at 65° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having beta-glucanase activity. In one aspect, a subsequence contains at least 1052 nucleotides of SEQ ID NO: 1 or the cDNA sequence thereof, at least 1037 nucleotides of SEQ ID NO: 1 or the cDNA sequence thereof, or 1022 nucleotides of SEQ ID NO: 1 or the cDNA sequence thereof).

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g., originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Variant: The term “variant” means a polypeptide having beta-glucanase activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1-3 amino acids adjacent to an amino acid occupying a position. The variants of the present invention have at least 20%, e.g., at least 40%, at least 50%, at least 60%, at least 70%, at least 80%, at least 90%, at least 95%, or at least 100% of the beta-glucanase activity of the polypeptide of sequence selected from the group consisting of: SEQ ID NO: 3, SEQ ID NO: 6, SEQ ID NO: 9, SEQ ID NO: 12, SEQ ID NO: 15, SEQ ID NO: 18 or the mature polypeptide of sequence selected from the group consisting of: SEQ ID NO: 2, SEQ ID NO: 5, SEQ ID NO: 8, SEQ ID NO: 11, SEQ ID NO: 14, SEQ ID NO: 17.

Wild-type beta-glucanase: The term “wild-type” beta-glucanase means a beta-glucanase expressed by a naturally occurring microorganism, such as a bacterium, yeast, or filamentous fungus found in nature.

Wash performance: The term “wash performance” is defined herein as the ability of an enzyme or a blend of enzymes to remove stains present on an object to be cleaned during, e.g., wash or hard surface cleaning relative to the wash performance without one or more on the enzymes present.

### Nomenclature

For purposes of the present invention, brackets are used to indicate alternative amino acids (using their one letter codes) at a particular position in a sequence. For example, the nomenclature [F/W] means that the amino acid at this position may be a phenylalanine (Phe, F) or a tryptophan (Trp, W). Amino acids indicated within brackets using this nomenclature may be separated by a vertical line or in some instances no line, e.g., [F/W] can also be designated as [FW].

In some instance, a sequence motif includes more than one set of brackets, each of which independently represents a position in a sequence. Thus, G[F/W]GNXEX[Q/E]XY ((SEQ ID NO: 33) means that G, conservative amino acid, is in the first position; either of F or W are in the second position; G, conservative amino acid, is in the third position; N, conservative amino acid is in the fourth position, X, any amino acid, is in the fifth position; E, conservative amino acid is in the sixth position; X, any amino acid, is in the seventh position; either of Q or E are in the eighth position; X, any amino acid, is in the ninth position; and Y, conservative amino acid, is in the tenth position. The motif represented by this designation may then be any of GFGNXEXQXY (SEQ ID NO: 34), GWGNXEXQXY (SEQ ID NO: 36), GFGNXEXEXY (SEQ ID NO: 35), GWGNXEXEXY (SEQ ID NO: 37).

Unless otherwise limited further, the amino acid X (or Xaa) is used herein to represent any of the 20 natural amino acids.

### DETAILED DESCRIPTION OF THE INVENTION

This invention provides the use of polypeptides having beta-glucanase activity and optionally one or more polypeptides having amylase, such as alpha-amylase, activity (and/or one or more polypeptides having protease activity) for cleaning or detergent compositions which have a benefit in removing stains and which can be used in cleaning or detergent applications or for processes such as cleaning hard-surfaces, dish wash and laundering. The invention also provides the use of beta-glucanases that are wash stable in detergent formulations in the presence of amylases. The polypeptide having beta-glucanase activity of the invention may show synergistic effect with one or more polypeptides having amylase, such as alpha-amylase activity (and/or one or more polypeptides having protease activity), e.g., wherein a preferred method for determining the REM synergistic effect is disclosed in the Examples, e.g., Example 7.).

### Polypeptides Having Beta-Glucanase Activity

Polypeptides useful according to the invention are those having beta-glucanase activity, which are of the laminarinase clade comprising GH16 glycoside hydrolase family polypeptides of bacterial origin from the order Bacillales having laminarinase activity and comprising certain conserved polypeptide motifs.

In one embodiment, the beta-glucanases comprise one or more or all of the conserved polypeptide motifs GEXDXME (SEQ ID NO: 28), GXGNXEXXXY (SEQ ID NO: 29), NXAXGG (SEQ ID NO: 30) and YTS[G/A][K/R](SEQ ID NO: 31).

For example, one shared motif of the beta-glucanases comprises the GEXDXME (SEQ ID NO: 28) motif. The two glutamic acid (E) residues of the GEXDXME (SEQ ID NO: 28) motif are analogous to residues E132 and E137 of SEQ ID: 27 which are directly involved as catalytic residues and are essential for catalysis. Also relevant is the GEIDIME (SEQ ID NO: 32) motif.

As another example, one shared motif of the beta-glucanases comprises the GXGNXEXXXY (SEQ ID NO: 29) motif. The asparagine (N) and glutamic acid(E) residues in the GXGNXEXXXY (SEQ ID NO: 29) motif are analogous to residue N45 and E47 in SEQ ID: 27 which may form direct or water-mediated hydrogen bonds to the laminarin substrate aiding in positioning the substrate correctly in the catalytic groove of the enzyme. Also relevant is the G[F/W]GNXEX[Q/E]XY (SEQ ID NO: 33) motif, which includes the motifs GFGNXEXQXY (SEQ ID NO: 34), GFGNXEXEXY (SEQ ID NO: 35), GWGNXEXQXY (SEQ ID NO: 36), and GWGNXEXEXY (SEQ ID NO: 37).

Another exemplary motif of the beta-glucanases comprises the NXAXGG (SEQ ID NO: 30) motif. The asparagine residue (N) in the NXAXGG (SEQ ID NO: 30) motif is analogous to the N225 residue in SEQ ID: 27 which is suggested to have a role in substrate binding. Also relevant is the [L/M]NXAXGG (SEQ ID NO: 42) motif, which includes both the LNXAXGG (SEQ ID NO: 43) and MNXAXGG (SEQ ID NO: 44) motifs.

Another motif of the beta-glucanases comprises the YTS[G/A][K/R](SEQ ID NO: 31) motif. The arginine or lysine in the YTS[G/A][K/R](SEQ ID NO: 31) motif carries a positive charge and this charge is proposed to be important for correct substrate interaction either through direct or water mediated interaction and is analogous to the R85 residue of the SEQ ID: 27. Thus, the YTS[G/A][K/R](SEQ ID NO: 31) motif also includes YTSGK (SEQ ID NO: 38), YTSGR (SEQ ID NO: 39), YTSAK (SEQ ID NO: 40), and YTSAR (SEQ ID NO: 41).

Further details are provided in Example 3, herein.

In one embodiment, the polypeptide is not the polypeptide of GENESEQP: BDR33035 or GENESEQP: AAB99272.

In one embodiment, the polypeptide has laminarinase (EC 3.2.1.6, EC 3.2.1.39, and/or EC 3.2.1.58) enzymatic activity, particularly endo-laminarinase activity EC 3.2.1.6 or EC 3.2.1.39 and eve more particularly EC 3.2.1.6 activity.

In one embodiment, the polypeptide is without cellulase activity (e.g., not having endo-cellulase activity on β-1,4 linkages between D-glucose units.

One embodiment relates to polypeptides having beta-glucanase activity, and comprising one or more of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31).

Polypeptides having beta-glucanase activity and comprising one or more, or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31) are particularly useful in cleaning compositions, processes, and uses, for example laundry and dishwash. In one embodiment, the polypeptide is selected from the group consisting of a polypeptide comprising, consisting, or consisting essentially of an amino acid sequence shown in SEQ ID NO: 3, SEQ ID NO: 6, SEQ ID NO: 9, SEQ ID NO: 12, SEQ ID NO: 15, SEQ ID NO: 18, or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto, and wherein the polypeptide further comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31).

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is selected from the group consisting of
(a) a polypeptide having at least 80% sequence identity to the polypeptide of SEQ ID NO: 3,
(b) a polypeptide having at least 98.5% sequence identity to the polypeptide of SEQ ID NO: 6,
(c) a polypeptide having at least 86% sequence identity to the polypeptide of SEQ ID NO: 9,
(d) a polypeptide having at least 97.5% sequence identity to the polypeptide of SEQ ID NO: 12,
(e) a polypeptide having at least 90% sequence identity to the polypeptide of SEQ ID NO: 15, and
(f) a polypeptide having at least 93% sequence identity to the polypeptide of SEQ ID NO: 18.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 3, or a polypeptide having at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 6, or a polypeptide having at least 98.5%, at least 99%, at least 99.5% or 100% sequence identity thereto.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 9, or a polypeptide having at least 86%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 12, or a polypeptide having at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 15, or a polypeptide having at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEXDXME (SEQ ID NO: 28), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 18, or a polypeptide having at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 3, or a polypeptide having at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 6, or a polypeptide having at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 9, or a polypeptide having at least 86%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 12, or a polypeptide having at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 15, or a polypeptide having at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% or 100% sequence identity thereto.

One embodiment of the invention relates to polypeptides having beta-glucanase activity, wherein the polypeptide comprises one or more or even all of the motifs GEIDIME (SEQ ID NO: 32), GXGNXEXXXY (SEQ ID NO: 29), NXAXGG (SEQ ID NO: 30), and YTS[G/A][K/R](SEQ ID NO: 31), and wherein the polypeptide is the polypeptide shown in SEQ ID NO: 18, or a polypeptide having at least 93%, at least 94%, at least 95%, at least 96%, at least 96.5%, at least 97%, at least 97.5%, at least 98%, at least 98.5%, at least 99%, at least 99.5% or 100% sequence identity thereto.

In an embodiment, the present invention relates to polypeptides having a sequence identity to a mature polypeptide of the sequence selected from the group consisting of: SEQ ID NO: 2, SEQ ID NO: 5, SEQ ID NO: 8, SEQ ID NO: 11, SEQ ID NO: 14, SEQ ID NO: 17; at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have beta-glucanase activity. In one aspect, the polypeptides differ by up to 10 amino acids, e.g., 1, 2, 3, 4, 5, 6, 7, 8, 9, or 10, from the mature polypeptide of the sequence selected from the group consisting of: SEQ ID NO: 2, SEQ ID NO: 5, SEQ ID NO: 8, SEQ ID NO: 11, SEQ ID NO: 14, SEQ ID NO: 17.

In an embodiment, the present invention relates to polypeptides having beta-glucanase activity, wherein said polypeptides having a sequence identity to the polypeptide of the sequence selected from the group consisting of: SEQ ID NO: 3, SEQ ID NO: 6, SEQ ID NO: 9, SEQ ID NO: 12, SEQ ID NO: 15, SEQ ID NO: 18; at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have beta-glucanase activity. In one aspect, the polypeptides differ by up to 10 amino acids, e.g., 1, 2, 3, 4, 5, 6, 7, 8, 9, or 10, from the polypeptide of SEQ ID NO: 3, SEQ ID NO: 6, SEQ ID NO: 9, SEQ ID NO: 12, SEQ ID NO: 15, SEQ ID NO: 18.

The polypeptide preferably comprises, consists essentially of, or consists of the amino acid sequence of SEQ ID NO: 2, SEQ ID NO: 5, SEQ ID NO: 8, SEQ ID NO: 11, SEQ ID NO: 14, SEQ ID NO: 17 or the mature polypeptide thereof; or is a fragment thereof having beta-glucanase activity. In one aspect, the mature polypeptide is SEQ ID NO: 3, SEQ ID NO: 6, SEQ ID NO: 9, SEQ ID NO: 12, SEQ ID NO: 15, or SEQ ID NO: 18.

In another embodiment, the present invention relates to a polypeptide having beta-glucanase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence selected from the group consisting of: SEQ ID NO: 1, SEQ ID NO: 4, SEQ ID NO: 7, SEQ ID NO: 10, SEQ ID NO: 13, SEQ ID NO: 16, (ii) the cDNA sequence thereof, or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

The polynucleotide of sequence selected from the group consisting of: SEQ ID NO: 1, SEQ ID NO: 4, SEQ ID NO: 7, SEQ ID NO: 10, SEQ ID NO: 13, SEQ ID NO: 16 or a subsequence thereof, as well as the polypeptide of sequence selected from the group consisting of: SEQ ID NO: 2, SEQ ID NO: 5, SEQ ID NO: 8, SEQ ID NO: 11, SEQ ID NO: 14, SEQ ID NO: 17 or a fragment thereof may be used to design nucleic acid probes to identify and clone DNA encoding polypeptides having beta-glucanase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵5, biotin, or avidin). Such probes are encompassed by the present invention.

A genomic DNA or cDNA library prepared from such other strains may be screened for DNA that hybridizes with the probes described above and encodes a polypeptide having beta-glucanase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with sequence selected from the group consisting of: SEQ ID NO: 1, SEQ ID NO: 4, SEQ ID NO: 7, SEQ ID NO: 10, SEQ ID NO: 13, SEQ ID NO: 16 or a subsequence thereof, the carrier material is used in a Southern blot.

For purposes of the present invention, hybridization indicates that the polynucleotide hybridizes to a labeled nucleic acid probe corresponding to (i) sequence selected from the group consisting of: SEQ ID NO: 1, SEQ ID NO: 4, SEQ ID NO: 7, SEQ ID NO: 10, SEQ ID NO: 13, SEQ ID NO: 16; (ii) the mature polypeptide coding sequence selected from the group consisting of: SEQ ID NO: 1, SEQ ID NO: 4, SEQ ID NO: 7, SEQ ID NO: 10, SEQ ID NO: 13, SEQ ID NO: 16; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 109 to 1548 or nucleotides 1 to 1548 of SEQ ID NO: 1. In one aspect, the nucleic acid probe is nucleotides 85 to 870 or nucleotides 1 to 870 of SEQ ID NO: 4. In one aspect, the nucleic acid probe is nucleotides 94 to 1248 or nucleotides 1 to 1248 of SEQ ID NO: 7. In one aspect, the nucleic acid probe is nucleotides 85 to 1230 or nucleotides 1 to 1230 of SEQ ID NO: 10. In one aspect, the nucleic acid probe is nucleotides 73 to 852 or nucleotides 1 to 852 of SEQ ID NO: 13. In one aspect, the nucleic acid probe is nucleotides 70 to 837 or nucleotides 70 to 837 of SEQ ID NO: 16.

In another aspect, the nucleic acid probe is a polynucleotide that encodes the polypeptide of sequence selected from the group consisting of: SEQ ID NO: 2, SEQ ID NO: 5, SEQ ID NO: 8, SEQ ID NO: 11, SEQ ID NO: 14, SEQ ID NO: 17; the mature polypeptide thereof; or a fragment thereof.

In another aspect, the nucleic acid probe is a sequence selected from the group consisting of: SEQ ID NO: 1, SEQ ID NO: 4, SEQ ID NO: 7, SEQ ID NO: 10, SEQ ID NO: 13, or SEQ ID NO: 16.

In another embodiment, the present invention relates to a polypeptide having beta-glucanase activity encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1, SEQ ID NO: 4, SEQ ID NO: 7, SEQ ID NO: 10, SEQ ID NO: 13, SEQ ID NO: 16.

In another embodiment, the present invention relates to variants of the mature polypeptide of sequence selected from the group consisting of: SEQ ID NO: 2, SEQ ID NO: 5, SEQ ID NO: 8, SEQ ID NO: 11, SEQ ID NO: 14, SEQ ID NO: 17, or the polypeptide of SEQ ID NO: 3, SEQ ID NO: 6, SEQ ID NO: 9, SEQ ID NO: 12, SEQ ID NO: 15, SEQ ID NO: 18 comprising a substitution, deletion, and/or insertion at one or more (e.g., several) positions. In an embodiment, the number of amino acid substitutions, deletions and/or insertions introduced into the mature polypeptide of sequence selected from the group consisting of: SEQ ID NO: 2, SEQ ID NO: 5, SEQ ID NO: 8, SEQ ID NO: 11, SEQ ID NO: 14, SEQ ID NO: 17, or the polypeptide of SEQ ID NO: 3, SEQ ID NO: 6, SEQ ID NO: 9, SEQ ID NO: 12, SEQ ID NO: 15, SEQ ID NO: 18 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for beta-glucanase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

In an embodiment, the polypeptide having beta-glucanase activity is isolated and/or purified.

In another embodiment, a pH optimum of a polypeptide having beta-glucanase activity is selected in the range from about 6 to about 9. In another embodiment a pH optimum of a polypeptide(s) of the present invention is selected from the group consisting of: 6, 6.5, 7, 7.5, 8, 8.5, 9. In another embodiment a pH optimum of a polypeptide(s) of the present invention is at least 6 (or at least 6.5, or at least 7, or at least 7.5, or at least 8, or at least 8.5, or at least 9). In another embodiment a pH optimum of a polypeptide(s) of the present invention is more than 6 (or more than 6.5, or more than 7, or more than 7.5, or more than 8, or more than 8.5, or more than 9).

In another embodiment, the polypeptide having beta-glucanase activity comprises alkaline beta-glucanase activity (e.g., beta-glucanase activity in an aqueous solution at pH 7.5 or above, e.g., beta-glucanase activity at pH selected from the group consisting of 7.5, 8, 9, 10, 11, 12, 13, 13.5, e.g., beta-glucanase activity at pH in the range from about 7.5 to about 13.5, wherein said aqueous solution optionally comprises a bleaching agent, preferably said pH is selected in the range from about 7.5 to about 12.5, further preferably said pH is selected in the range from about 8.5 to about 11.5, most preferably said pH is selected in the range from about 9.5 to about 10.5).

In another embodiment a beta-glucanase of the present invention is capable of:
i) having beta-glucanase activity for at least 15 minutes in an aqueous solution with a pH selected in the range from about 7.5 to about 13.5, wherein said aqueous solution optionally comprises a bleaching agent, preferably said pH is selected in the range from about 7.5 to about 12.5, further preferably said pH is selected in the range from about 8.5 to about 11.5, most preferably said pH is selected in the range from about 9.5 to about 10.5; and/or
ii) having beta-glucanase activity for at least 15 minutes in an aqueous solution at a temperature selected in the range from about 20° C. to about 75° C., wherein said aqueous solution optionally comprises a bleaching agent.

In another embodiment a beta-glucanase of the present invention is capable of having beta-glucanase activity in an aqueous solution at a temperature selected in the range from about 20° C. to about 75° C., wherein said aqueous solution optionally comprises a bleaching agent, preferably said temperature is selected in the range from about 40° C. to about 60° C. In another embodiment a beta-glucanase of the present invention is capable of having beta-glucanase activity in an aqueous solution at a temperature selected from the group consisting of: 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C.

In another embodiment a beta-glucanase of the present invention is capable of having beta-glucanase activity for at least 15 minutes, preferably at least 30 minutes. In another embodiment a beta-glucanase of the present invention is capable of having beta-glucanase activity for a period of time selected from the group consisting of: at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 minutes, e.g., in combination with any single or multiple embodiments as disclosed herein.

### Sources of Polypeptides Having Beta-Glucanase Activity

A polypeptide having beta-glucanase activity of the present invention may be obtained from microorganisms of any genus (e.g., Bacillus). For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. Preferably, the polypeptide is a gram-positive bacterial polypeptide of order Bacillales. For example, the polypeptide may be a Gram-positive bacterial polypeptide such as a Bacillus, Cohnella, Geobacillus, Oceanobacillus, Paenibacillus, Staphylococcus, or Thermobacillus polypeptide having beta-glucanase activity.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus sp., Bacillus akibai, Bacillus agaradhaerens, Bacillus mojavensis or Bacillus thuringiensis polypeptide.

In one aspect, the polypeptide is a Paenibacillus sp., Paenibacillus alvei, Paenibacillus amylolyticus, Paenibacillus glycanilyticus, Paenibacillus macerans, Paenibacillus pabuli, Paenibacillus polymyxa, or Paenibacillus xylanilyticus polypeptide.

In one aspect, the polypeptide is a Cohnella sp., Cohnella thermotolerans or Cohnella hongkongensis polypeptide.

In one aspect, the polypeptide is a Thermobacillus sp. polypeptide.

In another aspect, the polypeptide is not a fungal polypeptide (e.g., a polypeptide of the present invention excludes fungal polypeptides). An embodiment of the present invention is a composition (e.g., a cleaning or detergent composition) comprising said beta-glucanase polypeptide and one or more amylases (and/or one or more proteases).

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Catalytic Domains

In one embodiment, the polypeptide according to the invention comprises a catalytic domain, which can itself be used in the compositions, methods and uses described herein (e.g., alone or as part of the mature polypeptide).

In one embodiment, the present invention also relates to catalytic domains having a sequence identity to amino acids 1 to 251 of SEQ ID NO: 2 or SEQ ID NO: 3 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 8 to 251 of SEQ ID NO: 2 or SEQ ID NO: 3. The catalytic domain preferably comprises, consists essentially of, or consists of amino acids 1 to 251 of SEQ ID NO: 2 or SEQ ID NO: 3 or an allelic variant thereof; or is a fragment thereof having beta-glucanase activity.

In one embodiment, the present invention also relates to catalytic domains having a sequence identity to amino acids 1 to 248 of SEQ ID NO: 5 or SEQ ID NO: 6 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 10 to 248 of SEQ ID NO: 5 or SEQ ID NO: 6. The catalytic domain preferably comprises, consists essentially of, or consists of amino acids 1 to 248 of SEQ ID NO: 5 or SEQ ID NO: 6 or an allelic variant thereof; or is a fragment thereof having beta-glucanase activity.

In one embodiment, the present invention also relates to catalytic domains having a sequence identity to amino acids 1 to 233 of SEQ ID NO: 8 or SEQ ID NO: 9 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 4 to 233 of SEQ ID NO: 8 or SEQ ID NO: 9. The catalytic domain preferably comprises, consists essentially of, or consists of amino acids 1 to 233 of SEQ ID NO: 8 or SEQ ID NO: 9 or an allelic variant thereof; or is a fragment thereof having beta-glucanase activity.

In one embodiment, the present invention also relates to catalytic domains having a sequence identity to amino acids 1 to 234 of SEQ ID NO: 11 or SEQ ID NO: 12 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 1 to 234 of SEQ ID NO: 11 or SEQ ID NO: 12. The catalytic domain preferably comprises, consists essentially of, or consists of amino acids 1 to 234 of SEQ ID NO: 11 or SEQ ID NO: 12 or an allelic variant thereof; or is a fragment thereof having beta-glucanase activity.

In one embodiment, the present invention also relates to catalytic domains having a sequence identity to amino acids 1 to 260 of SEQ ID NO: 14 or SEQ ID NO: 15 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 12 to 260 of SEQ ID NO: 14 or SEQ ID NO: 15. The catalytic domain preferably comprises, consists essentially of, or consists of amino acids 1 to 260 of SEQ ID NO: 14 or SEQ ID NO: 15 or an allelic variant thereof; or is a fragment thereof having beta-glucanase activity.

In one embodiment, the present invention also relates to catalytic domains having a sequence identity to amino acids 1 to 256 of SEQ ID NO: 17 or SEQ ID NO: 18 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 10 to 256 of SEQ ID NO: 17 or SEQ ID NO: 18. The catalytic domain preferably comprises, consists essentially of, or consists of amino acids 1 to 256 of SEQ ID NO: 17 or SEQ ID NO: 18 or an allelic variant thereof; or is a fragment thereof having beta-glucanase activity.

### Binding Modules

In one embodiment, the polypeptide according to the invention comprises a binding module, such as a carbohydrate binding module (CBM), which can itself be used in the compositions, methods and uses described herein (e.g., alone or as part of the mature polypeptide).

In one embodiment, the present invention also relates to carbohydrate binding module having a sequence identity to amino acids 259 to 380, or amino acids 397 to 480 of SEQ ID NO: 2 or SEQ ID NO: 3 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the carbohydrate binding module comprise amino acid sequences that differ by up to 10 amino acids, e.g., 1, 2, 3, 4, 5, 6, 7, 8, 9, or 10, from amino acids 259 to 380, or amino acids 397 to 480 of SEQ ID NO: 2 or SEQ ID NO: 3. The CBM preferably comprises, consists essentially of, or consists of amino acids 259 to 380 or amino acids 397 to 480 of SEQ ID NO: 2 or SEQ ID NO: 3; or is a fragment thereof having carbohydrate binding activity.

In one embodiment, the present invention also relates to carbohydrate binding module having a sequence identity to amino acids 245 to 385 of SEQ ID NO: 8 or SEQ ID NO: 9 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the carbohydrate binding module comprise amino acid sequences that differ by up to 10 amino acids, e.g., 1, 2, 3, 4, 5, 6, 7, 8, 9, or 10, from amino acids 245 to 385 of SEQ ID NO: 8 or SEQ ID NO: 9. The CBM preferably comprises, consists essentially of, or consists of amino acids 245 to 385 of SEQ ID NO: 8 or SEQ ID NO: 9, or is a fragment thereof having carbohydrate binding activity.

In one embodiment, the present invention also relates to carbohydrate binding module having a sequence identity to amino acids 240 to 382 of SEQ ID NO: 11 or SEQ ID NO: 12 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one aspect, the carbohydrate binding module comprise amino acid sequences that differ by up to 10 amino acids, e.g., 1, 2, 3, 4, 5, 6, 7, 8, 9, or 10, from amino acids 240 to 382 of SEQ ID NO: 11 or SEQ ID NO: 12. The CBM preferably comprises, consists essentially of, or consists of amino acids 240 to 382 of SEQ ID NO: 11 or SEQ ID NO: 12, or is a fragment thereof having carbohydrate binding activity.

A carbohydrate binding module of the present invention may be applied in a fusion protein comprising at least one carbohydrate binding module operably linked to a catalytic domain. The catalytic domain may be from a hydrolase, isomerase, ligase, lyase, oxidoreductase, or transferase, aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase. The polynucleotide encoding the catalytic domain may be obtained from any prokaryotic, eukaryotic, or other source.

The polypeptides may further comprise a linker between the catalytic domain and the carbohydrate binding module.

### Polynucleotides

The present invention also relates to polynucleotides encoding a polypeptide, a catalytic domain, or carbohydrate binding module of the present invention, as described herein. In an embodiment, the polynucleotide encoding the polypeptide, catalytic domain, or carbohydrate binding module of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of sequence selected from the group consisting of: SEQ ID NO: 1, SEQ ID NO: 4, SEQ ID NO: 7, SEQ ID NO: 10, SEQ ID NO: 13, SEQ ID NO: 16, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces 35 cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57:109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMf31 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus sp-62449, Bacillus akibai, Bacillus agaradhaerens, Bacillus mojavensis and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8^{th} edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention (e.g., in vitro or ex vivo methods of production), comprising (a) cultivating a cell, which in its wild-type form produces the polypeptide, under conditions conducive for production of the polypeptide; and optionally, (b) recovering the polypeptide. In one aspect, the cell is a Bacillus cell. In another aspect, the cell is a Thermobacillus species, a Paenibacillus species, a Cohnella species, or a Bacillus species cell.

The present invention also relates to methods of producing a polypeptide of the present invention (e.g., in vitro or ex vivo methods of production), comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Production in Plants

The present invention also relates to isolated plants, e.g., a transgenic plant, plant part, or plant cell, comprising a polynucleotide of the present invention so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems.

Plant cells and specific plant cell compartments, such as chloroplasts, apoplasts, mitochondria, vacuoles, peroxisomes and cytoplasm are also considered to be a plant part.

Also included within the scope of the present invention are the progeny of such plants, plant parts, and plant cells.

The transgenic plant or plant cell expressing the polypeptide or domain may be constructed in accordance with methods known in the art. In short, the plant or plant cell is constructed by incorporating one or more expression constructs encoding the polypeptide or domain into the plant host genome or chloroplast genome and propagating the resulting modified plant or plant cell into a transgenic plant or plant cell.

The present invention also relates to methods of producing a polypeptide(s) or domain of the present invention comprising (a) cultivating a transgenic plant or a plant cell comprising a polynucleotide encoding the polypeptide or domain under conditions conducive for production of the polypeptide or domain; and (b) recovering the polypeptide or domain.

### Fermentation Broth Formulations

The present invention also relates to a fermentation broth formulation comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed fermentation broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed fermentation broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed fermentation broth or composition may contain the unfractionated contents of the fermentation materials derived at the end of the fermentation. Typically, the cell-killed fermentation brothor composition contains the spent culture medium and cell debris present after the microbial cells (e.g., filamentous fungal cells) are grown to saturation, incubated under carbon-limiting conditions to allow protein synthesis. In some embodiments, the cell-killed fermentation broth or composition contains the spent cell culture medium, extracellular enzymes, and killed filamentous fungal cells. In some embodiments, the microbial cells present in the cell-killed fermentation broth or composition can be permeabilized and/or lysed using methods known in the art.

A fermentation broth as described herein is typically a liquid, but may contain insoluble components, such as killed cells, cell debris, culture media components, and/or insoluble enzyme(s). In some embodiments, insoluble components may be removed to provide a clarified liquid composition.

The fermentation broth formulations and cell compositions of the present invention may be produced by a method described in WO 90/15861 or WO 2010/096673.

### Enzyme Compositions

The present invention also relates to compositions comprising a polypeptide(s) of the present invention. An embodiment is a cleaning or detergent composition comprising a beta-glucanase polypeptide of the invention and one or more amylases (and/or one or more proteases). Preferably, the compositions are enriched in such a polypeptide. The term “enriched” indicates that the beta-glucanase activity of the composition has been increased, e.g., with an enrichment factor of at least 1.1.

The compositions may comprise a polypeptide(s) of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

One embodiment relates to a composition, such as a cleaning composition comprising a beta-glucanase and an additional enzyme, which may be an amylase, a protease, a cellulase, a Dnase, a lipase, a mannanase, a pectinase, a protease, or a combination thereof.

### Other Enzymes

In one embodiment, a beta-glucanase of the invention is combined with one or more enzymes, such as at least two enzymes, more preferred at least three, four or five enzymes. Preferably, the enzymes have different substrate specificity, e.g., proteolytic activity, amylolytic activity, lipolytic activity, hemicellulytic activity or pectolytic activity. The detergent additive as well as the detergent composition may comprise one or more enzymes such as a protease, lipase, cutinase, an amylase, licheninase, carbohydrase, cellulase, pectinase, mannanase, arabinase, galactanase, xylanase, oxidase, e.g., a laccase and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases: Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g., T. reesei or T. viride. Other suitable cellulases are from Thielavia e.g., Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 495257, EP 531372, WO 96/11262, WO 96/29397, WO 98/08940. Other examples are cellulase variants such as those described in WO 94/07998, EP 531315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 01/62903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

Mannanases: Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/64619. A commercially available mannanase is Mannaway (Novozymes A/S).

Proteases: Suitable proteases include those of bacterial, fungal, plant, viral or animal origin, e.g., vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from, e.g., family M4 or other metalloprotease such as those from M5, M7 or M8 families.

The term “subtilases” refers to a sub-group of serine protease according to Siezen et al., 1991, Protein Engng. 4: 719-737 and Siezen et al., 1997, Protein Science 6: 501-523. Serine proteases are a subgroup of proteases characterized by having a serine in the active site, which forms a covalent adduct with the substrate. The subtilases may be divided into 6 sub-divisions, i.e., the Subtilisin family, the Thermitase family, the Proteinase K family, the Lantibiotic peptidase family, the Kexin family and the Pyrolysin family.

Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO 2009/021867, and Subtilisin lentus, Subtilisin Novo, subtilisin Carlsberg, Bacillus licheniformis, subtilisin BPN′, subtilisin 309, subtilisin 147 and subtilisin 168 and, e.g., protease PD138 described in (WO 93/18140). Other useful proteases may be those described in WO 01/016285 and WO 02/016547. Examples of trypsin-like proteases are trypsin (e.g., of porcine or bovine origin) and the Fusarium protease described in WO 94/25583 and WO 2005/040372, and the chymotrypsin proteases derived from Cellumonas described in WO 2005/052161 and WO 2005/052146.

A further preferred protease is the alkaline protease from Bacillus lentus DSM 5483, as described for example in WO 95/23221, and variants thereof which are described in WO 92/21760, WO 95/23221, EP 1921147 and EP 1921148.

Examples of metalloproteases are the neutral metalloprotease as described in WO 2007/044993 (Proctor & Gamble/Genencor Int.) such as those derived from Bacillus amyloliquefaciens.

Examples of useful proteases are the variants described in: WO 89/06279, WO 92/19729, WO 96/34946, WO 98/20115, WO 98/20116, WO 99/11768, WO 01/44452, WO 2003/006602, WO 2004/03186, WO 2004/041979, WO 2007/006305, WO 2011/036263, WO 2011/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO 1 of WO 2016/001449. More preferred the protease variants may comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V199I, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease shown in SEQ ID NO 1 of WO 2016/001449, the Bacillus amylolichenifaciens protease (BPN′) shown in SEQ ID NO 2 of WO 2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO 2004/067737, wherein said protease variant has a sequence identity of at least 75% but less than 100% to SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity®100T, Blaze Evity®125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases: Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/087508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/56782), perhydrolases from the CE 7 family (WO 2009/67279), and variants of the M. smegmatis perhydrolase in particular the S54V variant used in the commercial product Gentle Power Bleach from Huntsman Textile Effects Pte Ltd (WO 2010/100028).

Amylases: Suitable amylases which can be used together with beta-glucanase of the invention may be an alpha-amylase or a glucoamylase and may be of bacterial or fungal origin. Chemically modified or protein engineered variants are included. Amylases include, for example, alpha-amylases obtained from Bacillus, e.g., a special strain of Bacillus licheniformis, described in more detail in GB 1,296,839. Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444. Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions: M197T;

$$\begin{matrix}H 156 Y + A 181 T + N190 F + A 209 V + Q 264 S ; \\ G 48 A + T 49 I + G 107 A + H 156 Y + A 181 T + N 190 F + I 201 F + A 209 V + Q 264 S .\end{matrix}$$

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184. Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476. Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264. Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

$$\begin{matrix}N 128 C + K 178 L + T 182 G + Y 305 R + G 475 K ; \\ N 128 C + K 178 L + T 182 G + F 202 Y + Y 305 R + D 319 T + G 475 K ; \\ S 125 A + N 128 C + K 178 L + T 182 G + Y 305 R + G 475 K ; or \\ S 125 A + N 128 C + T 131 I + T 165 I + K 178 L + T 182 G + Y 305 R + G 475 K\end{matrix}$$

wherein the variants are C-terminally truncated and optionally further comprises a substitution at position 243 and/or a deletion at position 180 and/or position 181. Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions. Other examples are amylase variants such as those described in WO 2011/098531, WO 2013/001078 and WO 2013/001087. Commercially available amylases are Duramyl™, Termamyl™, Fungamyl™, Stainzyme™, Stainzyme Plus™, Natalase™, Liquozyme X, BAN™, Amplify Prime® (from Novozymes A/S), and Rapidase™, Purastar™/Effectenz™, Powerase and Preferenz S100 (from Genencor International Inc./DuPont).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates as described above, liquids, in particular stabilized liquids, or slurries.

Nucleases: Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a fungi or bacterium. In particular, a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO 2014/087011 and WO 2017/060475. Particularly preferred is also a DNase obtainable from a species of Aspergillus; in particular a DNase which is obtainable from Aspergillus oryzae, such as a DNase described in WO 2015/155350.

### Exemplary Combinations

In one aspect, the beta-glucanase of the invention may be combined with at least two enzymes. These additional enzymes are described in details in the section “other enzymes”, more preferred at least three, four or five enzymes. Preferably, the enzymes have different substrate specificity, e.g., carbolytic activity, proteolytic activity, amylolytic activity, lipolytic activity, hemicellulytic activity or pectolytic activity. The enzyme combination may for example be a beta-glucanase of the invention with another stain removing enzyme, e.g., a beta-glucanase of the invention and a protease, a beta-glucanase of the invention and a serine protease, a beta-glucanase of the invention and an amylase, a beta-glucanase of the invention and a cellulase, beta-glucanase of the invention and a lipase, a beta-glucanase of the invention and a cutinase, a beta-glucanase of the invention and a mannanase, which is a GH5 mannananase and/or a GH26 mannanase, a beta-glucanase of the invention and a pectinase or a beta-glucanase of the invention and an anti-redeposition enzyme. More preferably, the beta-glucanase of the invention is combined with at least two other stain removing enzymes, e.g., a beta-glucanase of the invention, a lipase and an amylase; or a beta-glucanase of the invention, a protease and an amylase; or a beta-glucanase of the invention, a protease and a lipase; or a beta-glucanase of the invention, a protease and a pectinase; or a beta-glucanase of the invention, a protease and a cellulase; or a beta-glucanase of the invention, a protease and a hemicellulase; or a beta-glucanase of the invention, a protease and a cutinase; or a beta-glucanase of the invention, an amylase and a pectinase; or a beta-glucanase of the invention, an amylase and a cutinase; or a beta-glucanase of the invention, an amylase and a cellulase; or a beta-glucanase of the invention, an amylase and a hemicellulase; or beta-glucanase of the invention, an amylase and a mannanase, which is a GH5 mannananase and/or a GH26 mannanase; or beta-glucanase of the invention, a protease, and a mannanase, which is a GH5 mannananase and/or a GH26 mannanase, or beta-glucanase of the invention, a lipase and a mannanase, which is a GH5 mannananase and/or a GH26 mannanase; or a beta-glucanase of the invention, a pectinase, and a mannanase, which is a GH5 mannananase and/or a GH26 mannanase; or a beta-glucanase of the invention, a lipase and a pectinase; or a beta-glucanase of the invention, a lipase and a cutinase; or a beta-glucanase of the invention, a lipase and a cellulase; or a beta-glucanase of the invention, a lipase and a hemicellulase. Even more preferably, a beta-glucanase of the invention may be combined with at least three other stain removing enzymes, e.g., a beta-glucanase of the invention, a protease, a lipase and an amylase; or a beta-glucanase of the invention, a protease, an amylase and a pectinase; or a beta-glucanase of the invention, a protease, an amylase and a cutinase; or a beta-glucanase of the invention, a protease, an amylase and a cellulase; or a beta-glucanase of the invention, a protease, an amylase and a hemicellulase; or beta-glucanase of the invention, a protease, an amylase and a mannanase, which is a GH5 mannananase and/or a GH26 mannanase; or a beta-glucanase of the invention, a protease, a lipase and a mannanase, which is a GH5 mannananase and/or a GH26 mannanase; or a beta-glucanase of the invention, an amylase, a lipase, and a mannanase, which is a GH5 mannananase and/or a GH26 mannanase; or a beta-glucanase of the invention, a protease, a cutinase, and a mannanase, which is a GH5 mannananase and/or a GH26 mannanase; or a beta-glucanase of the invention, an amylase, a cutinase, and a mannanase, which is a GH5 mannananase and/or a GH26 mannanase; or a beta-glucanase of the invention, an amylase, a lipase and a pectinase; or a beta-glucanase of the invention, an amylase, a lipase and a cutinase; or a beta-glucanase of the invention, an amylase, a lipase and a cellulase; or a beta-glucanase of the invention, an amylase, a lipase and a hemicellulase; or a beta-glucanase of the invention, a protease, a lipase and a pectinase; or a beta-glucanase of the invention, a protease, a lipase and a cutinase; or a beta-glucanase of the invention, a protease, a lipase and a cellulase; or a beta-glucanase of the invention, a protease, a lipase and a hemicellulase. A beta-glucanase according to the present invention may be combined with any of the enzymes selected from the non-exhaustive list comprising: carbohydrases, such as an amylase, a hemicellulase, a mannanase, a pectinase, a cellulase, a xanthanase or a pullulanase, a peptidase, a protease or a lipase.

In a preferred embodiment, a beta-glucanase of the invention is combined with a serine protease, e.g., an S8 family protease such as Savinase®.

In another embodiment of the present invention, a beta-glucanase of the invention may be combined with one or more metalloproteases, such as an M4 metalloprotease, including Neutrase® or Thermolysin. Such combinations may further comprise combinations of the other detergent enzymes as outlined above.

The cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one beta-glucanase of the invention. The cleaning process or a textile care process can for example be carried out in a machine washing process or in a manual washing process. The washing solution can for example be an aqueous washing solution containing a detergent composition.

The fabrics and/or garments subjected to a washing, cleaning or textile care process of the present invention may be conventional washable laundry, for example household laundry. Preferably, the major part of the laundry is garments and fabrics, including knits, woven, denims, non-woven, felts, yarns, and towelling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

Typical detergent compositions includes various components in addition to the enzymes, these components have different effects, some components like the surfactants lower the surface tension in the detergent, which allows the stain being cleaned to be lifted and dispersed and then washed away, other components like bleach systems removes discolor often by oxidation and many bleaches also have strong bactericidal properties, and are used for disinfecting and sterilizing. Yet other components like builder and chelator softens, e.g., the wash water by removing the metal ions from the liquid.

In a particular embodiment, the invention concerns the use of a composition comprising a beta-glucanase of the invention, wherein said enzyme composition further comprises at least one or more of the following a surfactant, a builder, a chelator or chelating agent, bleach system or bleach component in laundry or dish wash.

In a preferred embodiment of the invention the amount of a surfactant, a builder, a chelator or chelating agent, bleach system and/or bleach component are reduced compared to amount of surfactant, builder, chelator or chelating agent, bleach system and/or bleach component used without the added beta-glucanase of the invention. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of beta-glucanase of the invention, such as a conventional amount of such component. In one aspect, the beta-glucanase of the invention is used in detergent compositions wherein said composition is free of at least one component which is a surfactant, a builder, a chelator or chelating agent, bleach system or bleach component and/or polymer.

### Compositions

In an embodiment, a polypeptide having beta-glucanase activity is combined with one or more polypeptides having amylase, e.g., alpha-amylase activity and/or one or more polypeptides having protease activity in a cleaning or detergent composition.

In an embodiment, the combination of a polypeptide having beta-glucanase activity and the one or more polypeptides having amylase (and/or one or more polypeptides having protease activity), preferably said polypeptide having beta-glucanase activity and said one or more amylases (and/or one or more proteases) in a cleaning or detergent composition have a synergistic effect; further preferably said synergistic effect is a REM synergistic effect, further most preferably said REM synergistic effect is of more than 6.5 at about 40° C. for about 30 minutes at pH of about 7.5, further most preferably said REM synergistic effect is of more than 6.1 at about 40° C. for about 30 minutes at pH of about 10, further most preferably said REM synergistic effect is of more than 6.2 at about 40° C. for about 30 minutes at pH of about 10. In particular, a cleaning or detergent composition comprises a beta-glucanase polypeptide and one or more alpha-amylases and/or one or more proteases.

In another embodiment a polypeptide(s) having beta-glucanase activity and one or more amylases (and/or one or more proteases) in a cleaning or detergent composition have a synergistic effect; preferably said synergistic effect is a REM synergistic effect, further preferably said REM synergistic effect is of more than 6.5 at about 40° C. for about 30 minutes at pH of about 7.5, further preferably said REM synergistic effect is of more than 6.1 at about 40° C. for about 30 minutes at pH of about 10, further preferably said REM synergistic effect is of more than 6.2 at about 40° C. for about 30 minutes at pH of about 10.

In another embodiment REM synergistic effect is of more than 1.4 (such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0) at about 40° C. (or 35° C., 45° C., 50° C., 55° C., 60° C.) for about 30 minutes (or 15 min, 20 min, 25 min, 35 min, 40 min) at pH of about 7.0 (or 7.5, 8.0, 8.5, 9.0, 9.5, 10.0, 10.5, 11.0, 11.5, 12.0, 12.5, 13.0, 13.5), e.g., in Wascator bottle wash in Model detergent A at 40° C., 30 min (pH 7.7), or Wascator bottle wash in Model detergent X at 40° C., 30 min (pH 10.1), or Wascator bottle wash in ADW Model detergent A at 40° C., 30 min (pH 10.2) (e.g., see Example 7).

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 3 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 6 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 9 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 12 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 15 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more amylases, wherein said alpha-amylase is selected from the group consisting of:
a) a polypeptide having at least 90%, such as at least 95%, at least 96%, at least 97%, at least 98%, at least 99%, or even 100% sequence identity to SEQ ID NO: 45;
b) a polypeptide having at least 90%, such as at least 95%, at least 96%, at least 97%, at least 98%, at least 99%, or even 100% sequence identity to SEQ ID NO: 46;
c) a polypeptide having at least 90% sequence identity to SEQ ID NO: 47;
d) a polypeptide having at least 90%, such as at least 95%, at least 96%, at least 97%, at least 98%, at least 99%, or even 100% sequence identity to SEQ ID NO: 48;
e) a polypeptide having at least 90%, such as at least 95%, at least 96%, at least 97%, at least 98%, at least 99%, or even 100% sequence identity to SEQ ID NO: 48, wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444;
f) a polypeptide having at least 90%, such as at least 95%, at least 96%, at least 97%, at least 98%, at least 99%, or even 100% sequence identity to SEQ ID NO: 49;
g) a polypeptide having at least 90%, such as at least 95%, at least 96%, at least 97%, at least 98%, at least 99%, or even 100% sequence identity to the hybrid polypeptide of SEQ ID NO: 50;
h) a polypeptide having at least 90%, such as at least 95%, at least 96%, at least 97%, at least 98%, at least 99%, or even 100% sequence identity to the hybrid polypeptide of SEQ ID NO: 51, wherein the hybrid polypeptide comprises a substitution, a deletion or an insertion in one of more of positions: 48, 49, 107, 156, 181, 190, 197, 201, 209 and/or 264;
i) a polypeptide having at least 90%, such as at least 95%, at least 96%, at least 97%, at least 98%, at least 99%, or even 100% sequence identity to SEQ ID NO: 51;
j) a polypeptide having at least 90%, such as at least 95%, at least 96%, at least 97%, at least 98%, at least 99%, or even 100% sequence identity to SEQ ID NO: 51, wherein the polypeptide comprises a substitution, a deletion or an insertion in one of more of positions: 181, 182, 183, 184, 195, 206, 212, 216 and/or 269;
k) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54;
l) a polypeptide having at least 90%, such as at least 95%, at least 96%, at least 97%, at least 98%, at least 99%, or even 100% sequence identity to SEQ ID NO: 52, SEQ ID NO: 53 or SEQ ID NO: 54, wherein the polypeptide comprises a substitution, a deletion or an insertion in one of more of positions: 140, 183, 184 195, 206, 243, 260, 304 and/or 476;
m) a polypeptide having at least 90%, such as at least 95%, at least 96%, at least 97%, at least 98%, at least 99%, or even 100% sequence identity to SEQ ID NO: 55;
n) a polypeptide having at least 90%, such as at least 95%, at least 96%, at least 97%, at least 98%, at least 99%, or even 100% sequence identity to SEQ ID NO: 56;
o) a polypeptide having at least 90%, such as at least 95%, at least 96%, at least 97%, at least 98%, at least 99%, or even 100% sequence identity to SEQ ID NO: 56, wherein the polypeptide comprises a substitution, a deletion or an insertion in one of more of positions: 176, 177, 178, 179, 190, 201, 207, 211 and/or 264;
p) a polypeptide having at least 90%, such as at least 95%, at least 96%, at least 97%, at least 98%, at least 99%, or even 100% sequence identity to SEQ ID NO: 57;
q) a polypeptide having at least 90%, such as at least 95%, at least 96%, at least 97%, at least 98%, at least 99%, or even 100% sequence identity to SEQ ID NO: 57, wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475;
r) a polypeptide having at least 90%, such as at least 95%, at least 96%, at least 97%, at least 98%, at least 99%, or even 100% sequence identity to SEQ ID NO: 58, wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
s) a polypeptide having at least 90%, such as at least 95%, at least 96%, at least 97%, at least 98%, at least 99%, or even 100% sequence identity to SEQ ID NO: 59;
t) a variant of SEQ ID NO:58 having alterations G182*+D183*;
u) a variant of SEQ ID NO:60 having alterations H183*+G184*+I405L+A421H+A422P+A428T;
v) a variant of SEQ ID NO:59 having alterations M9L+R118K+G149A+G182T+G186A+D183*+G184*+N195F+M202L+T257I+Y295F+N299Y+R320K+M323T+A339S+E345R+R458K;
w) a variant of SEQ ID NO: 61 having alterations R178*+G179*+E187P+I203Y+R458N+T459S+D460T+G476K;
x) a variant of SEQ ID NO: 62 having alteration M202L;
y) a variant of SEQ ID NO: 63 having alterations R180*+S181*+S243Q+G475K;
z) a variant of SEQ ID NO: 64 having alterations D183*+G184*+W140Y+N195F+I206Y+Y243F+E260G+G304R+G476K;
aa) a variant of SEQ ID NO: 65 having alterations H1*+N54S+V56T+K72R+G109A+F113Q+R116Q+W167F+Q172G+A174S+G184T+N195F+V206L+K391A+P473R+G476K; and
bb) a variant of SEQ ID NO: 66 having alterations M9L+R118K+G149A+G182T+G186A+D183*+G184*+N195F+T246V+T257I+Y295F+N299Y+R320K+M323T+A339S+E345R+R458K.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more proteases, wherein said protease is selected from the group consisting of:
a) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 67;
b) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations Y161A+R164S+A188P;
c) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S97SE;
d) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9R+A15T+G59E+V66A+A188P+V199I+Q239R+N255D;
e) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to a variant of SEQ ID NO: 67 having alterations S9E+N42R+N74D+V199I+Q200L+Y203W+S253D+N255W+L256E; and
f) a polypeptide having at least 60% sequence identity to SEQ ID NO: 68;
g) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 69; and
h) a polypeptide having at least 60%, such as at least 65%, at least 70%, at least 75%, at least 80%, at least 85%, at least 90% at least 95%, at least 96%, at least 97%, at least 98%, at least 99%, or even 100% sequence identity to SEQ ID NO: 70.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more cellulases, and wherein the cellulase is selected from the group consisting of:
a) a cellulase having at least 60%, at least 65%, at least 70%, at least 75%, at least 80%, at least 85%, at least 90%, at least 95%, at least 98% or 100% sequence identity to SEQ ID NO: 71;
b) a cellulase having at least 60%, at least 65%, at least 70%, at least 75%, at least 80%, at least 85%, at least 90%, at least 95%, at least 98% or 100% sequence identity to SEQ ID NO: 72;
c) a cellulase having at least 60%, at least 65%, at least 70%, at least 75%, at least 80%, at least 85%, at least 90%, at least 95%, at least 98% or 100% sequence identity to SEQ ID NO: 73;
d) a cellulase having at least 60%, at least 65%, at least 70%, at least 75%, at least 80%, at least 85%, at least 90%, at least 95%, at least 98% or 100% sequence identity to SEQ ID NO: 74,
e) a cellulase having at least 60%, at least 65%, at least 70%, at least 75%, at least 80%, at least 85%, at least 90%, at least 95%, at least 98% or 100% sequence identity to SEQ ID NO: 75, and
f) a cellulase having at least 60%, at least 65%, at least 70%, at least 75%, at least 80%, at least 85%, at least 90%, at least 95%, at least 98% or 100% sequence identity to SEQ ID NO: 76.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more lipases, and wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 77 comprising one or more of the substitutions selected from the group consisting of D27R, G38A, G91A/Q, D96E, G163K, T231R, N233R, D254S and P256T, compared to SEQ ID NO: 77, wherein each position corresponds to the position in SEQ ID NO: 77.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more mannanases, and wherein the mannanase is selected from the group consisting of:
a) a mannanase, wherein the mannanase preferably belongs to the Glycoside Hydrolase Family 5 mannanases:
   i. a mannanase having at least 60%, at least 65%, at least 70%, at least 75%, at least 80%, at least 85%, at least 90%, at least 95%, at least 98% or 100% sequence identity to SEQ ID NO: 78;
   ii. a mannanase having at least 60%, at least 65%, at least 70%, at least 75%, at least 80%, at least 85%, at least 90%, at least 95%, at least 98% or 100% sequence identity to SEQ ID NO: 79; and
   iii. a mannanase having at least 60%, at least 65%, at least 70%, at least 75%, at least 80%, at least 85%, at least 90%, at least 95%, at least 98% or 100% sequence identity to SEQ ID NO: 80;
b) a mannanase wherein the mannanase preferably belongs to the Glycoside Hydrolase Family 26 mannanases:
   i. a mannanase having at least 60%, at least 65%, at least 70%, at least 75%, at least 80%, at least 85%, at least 90%, at least 95%, at least 98% or 100% sequence identity to SEQ ID NO: 81;
   ii. a mannanase having at least 60%, at least 65%, at least 70%, at least 75%, at least 80%, at least 85%, at least 90%, at least 95%, at least 98% or 100% sequence identity to SEQ ID NO: 82;
   iii. a mannanase having at least 60%, at least 65%, at least 70%, at least 75%, at least 80%, at least 85%, at least 90%, at least 95%, at least 98% or 100% sequence identity to SEQ ID NO: 83;
   iv. a mannanase having at least 60%, at least 65%, at least 70%, at least 75%, at least 80%, at least 85%, at least 90%, at least 95%, at least 98% or 100% sequence identity to SEQ ID NO: 84;
   v. a mannanase having at least 60%, at least 65%, at least 70%, at least 75%, at least 80%, at least 85%, at least 90%, at least 95%, at least 98% or 100% sequence identity to SEQ ID NO: 85;
   vi. a mannanase having at least 60%, at least 65%, at least 70%, at least 75%, at least 80%, at least 85%, at least 90%, at least 95%, at least 98% or 100% sequence identity to SEQ ID NO: 86;
   vii. a mannanase having at least 60%, at least 65%, at least 70%, at least 75%, at least 80%, at least 85%, at least 90%, at least 95%, at least 98% or 100% sequence identity to SEQ ID NO: 87;
   viii. a mannanase having at least 60%, at least 65%, at least 70%, at least 75%, at least 80%, at least 85%, at least 90%, at least 95%, at least 98% or 100% sequence identity to SEQ ID NO: 88;
   ix. a mannanase having at least 60%, at least 65%, at least 70%, at least 75%, at least 80%, at least 85%, at least 90%, at least 95%, at least 98% or 100% sequence identity to SEQ ID NO: 89;
   x. a mannanase having at least 60%, at least 65%, at least 70%, at least 75%, at least 80%, at least 85%, at least 90%, at least 95%, at least 98% or 100% sequence identity to SEQ ID NO: 90;
   xi. a mannanase having at least 60%, at least 65%, at least 70%, at least 75%, at least 80%, at least 85%, at least 90%, at least 95%, at least 98% or 100% sequence identity to SEQ ID NO: 91.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more pectinases, and wherein the pectinase has at least 60%, at least 65%, at least 70%, at least 75%, at least 80%, at least 85%, at least 90%, at least 95%, at least 98% or 100% sequence identity to SEQ ID NO: 92.

In another embodiment, a cleaning or detergent composition of the invention comprising a beta-glucanase polypeptide wherein the polypeptide comprises, consists, or consists essentially of a polypeptide having an amino acid sequence of SEQ ID NO: 18 or a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 96.5%, at least 97%, at least 97.5%, at least 98%, at least 98.5%, at least 99%, at least 99.5% identity thereto and one or more DNases, and wherein the DNase is selected from the group consisting of:
a) a DNAse having at least 60%, at least 65%, at least 70%, at least 75%, at least 80%, at least 85%, at least 90%, at least 95%, at least 98% or 100% sequence identity to SEQ ID NO: 93; and
b) a DNase having at least 60%, at least 65%, at least 70%, at least 75%, at least 80%, at least 85%, at least 90%, at least 95%, at least 98% or 100% sequence identity to SEQ ID NO: 94.

The present invention also relates to compositions comprising a beta-glucanase of the invention (e.g., a polypeptide(s) of the present invention). The present invention also relates to compositions comprising a beta-glucanase of the invention (e.g., a polypeptide(s) of the present invention) and one or more additional enzymes. The present invention also relates to compositions comprising a beta-glucanase of the invention (e.g., a polypeptide(s) of the present invention) and one or more amylases (and/or one or more proteases), preferably said one or more amylases is one or more alpha-amylases. An embodiment is a cleaning or detergent composition comprising a beta-glucanase polypeptide of the invention and one or more amylases (and/or one or more proteases).

In one embodiment, the present invention relates to compositions in particular to cleaning compositions and/or detergent compositions comprising a beta-glucanase of the invention and a suitable surfactant.

In one embodiment, the detergent composition may be adapted for specific uses such as laundry, in particular household laundry, dish washing or hard surface cleaning.

In another embodiment a composition of the present invention is a cleaning or a detergent composition.

Alkaline Liquid detergents having high pH are widely used in cleaning, such as laundry and dish wash cleaning. Liquid detergents with elevated pH are especially commonly used by consumers in North America. The high pH cleaning compositions are also used in industrial cleaning processes. Alkaline detergents include liquids having detergent properties. The pH of such detergents usually ranges in pH from 9 to 12.5. The high pH detergents typically comprise components such as surfactants, builders and bleach components and additionally they may also contain a significant amount of water and alkalis such as NaOH, TSP (Trisodium phosphate), ammonia, Sodium carbonate, Potassium hydroxide (KOH) these alkalis are usually added in amount corresponding to 0.1 to 30 percent weight (wt). Adding enzymes to detergents is highly advantageous as the specific activities of these enzymes effectively removes specific stains from surfaces such as textile and cutlery. However, the difficulty of maintaining acceptable enzyme stability in the high pH liquid detergents has for many years prohibited inclusion of enzymes into these detergents. In another embodiment the present invention relates high pH liquid cleaning compositions comprising an alkaline stable beta-glucanase of the present invention suitable for use in such compositions.

In another embodiment a composition of the present invention preferably contains alkaline buffer system to provide a pH of at least about 7.5, at least about 8, at least about 9, preferably pH 10 or above. Preferably the pH is from about 9 to about 13. In order to achieve the high pH it is necessary to have present an alkali metal hydroxide especially sodium or potassium hydroxide, normally in an amount of 0.1 to about 30% by weight (percentage by weight, abbreviated wt %) of the composition, and preferably 1.0 to 2.5%, or higher amounts of a suitable alkali metal silicate such as metal silicate, according to the desired pH for the product.

In another embodiment a composition of the present invention has pH 6.5 or above, preferably pH of 7.0 or above, more preferably pH of 7.5 or above and optionally comprises a bleaching agent; preferably said pH is selected in the range from about 7.5 to about 13.5, further preferably said pH is selected in the range from about 7.5 to about 12.5, most preferably said pH is selected in the range from about 8.5 to about 11.5, further most preferably said pH is selected in the range from about 9.5 to about 10.5. In a preferred embodiment, detergent compositions with such preferred pH-ranges are solid.

In another embodiment the present invention relates to a liquid cleaning composition having pH 6.5 or above, preferably pH 7.5 or above, comprising at least 0.001 (e.g., at least 0.01) wt % beta-glucanase, wherein said beta-glucanase has an amino acid sequence which has at least 60% sequence identity to the polypeptide of the sequence selected from the group consisting of: SEQ ID NO: 3, SEQ ID NO: 6, SEQ ID NO: 9, SEQ ID NO: 12, SEQ ID NO: 15, SEQ ID NO: 18. In further related embodiments beta-glucanase has an amino acid sequence which has at least 82% (or at least 80%, 83%, or 84%, or 85%, or 86%, or 87%, or 88%, or 89%, or 90%, or 91%, or 92%, or 93%, or 94%, or 95%, or 96%, or 97%, or 98% or 99% or 100%) sequence identity to the mature polypeptide of the sequence selected from the group consisting of: SEQ ID NO: 3, SEQ ID NO: 6, SEQ ID NO: 9, SEQ ID NO: 12, SEQ ID NO: 15, SEQ ID NO: 18.

The detergent compositions of the invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. The detergent compositions of the invention may find use in hard surface cleaning, automatic dishwashing applications, as well as cosmetic applications such as dentures, teeth, hair and skin. It can also be used to clean the parts of the dishwasher or washing machine interior during cleaning process, especially the hidden parts, like the water pipelines inside the machine, especially these in the rotatable arms, and the sieve/filter.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous.

Unless otherwise noted, all component or composition levels provided herein are made in reference to the active level of that component or composition, and are exclusive of impurities, for example, residual solvents or by-products, which may be present in commercially available sources.

The beta-glucanase of the invention is normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.75% of enzyme protein by weight of the composition, even more preferably at a level of from 0.001% to 0.5% of enzyme protein by weight of the composition.

Furthermore, the beta-glucanase of the invention is normally incorporated in the detergent composition in such amounts that their concentration in the wash water is at a level of from 0.0000001% to 1% enzyme protein, preferably at a level of from 0.000005% to 0.01% of enzyme protein, more preferably at a level of from 0.000001% to 0.005% of enzyme protein, even more preferably at a level of from 0.00001% to 0.001% of enzyme protein in wash water.

As is well known, the amount of enzyme will also vary according to the particular application and/or as a result of the other components included in the compositions.

A composition for use in automatic dishwash (ADW), for example, may include 0.0001%-50%, such as 0.001%-25%, such as 0.002%-20%, such as 0.01-15% of enzyme protein by weight of the composition. A composition for use in automatic dishwash (ADW), for example, may include 0.001%-50%, such as 0.01%-25%, such as 0.02%-20%, such as 0.1-15% of enzyme protein by weight of the composition.

A composition for use in laundry granulation, for example, may include 0.0001%-50%, such as 0.001%-20%, such as 0.01%-15%, such as 0.05%-10% of enzyme protein by weight of the composition.

A composition for use in laundry liquid, for example, may include 0.0001%-10%, such as 0.001-7%, such as 0.1%-5% of enzyme protein by weight of the composition.

A preferred detergent composition, comprises the polypeptide of the invention in concentrations of 0.00001 mg enzyme protein/g composition to 100 mg enzyme protein/g composition, preferred 0.0001 mg enzyme protein/g composition to 50 mg enzyme protein/g composition, more preferred 0.001 mg enzyme protein/g composition to 20 mg enzyme protein/g composition, especially preferred 0.01 mg enzyme protein/g composition to 10 mg enzyme protein/g composition.

A preferred detergent composition, especially a composition formulated as unit dose product, comprises the polypeptide of the invention in amounts from 0.01 mg/job to 100 mg enzyme protein/job, preferred 0.1 mg enzyme protein/job to 20 mg/job, more preferred 0.2 to 10 mg enzyme protein/job, especially preferred 0.3 to 5 mg enzyme protein/job. For example, amounts of 0.5 mg 1 mg, 1.5 mg, 2 mg or 2.5 mg enzyme protein/job can be used. The expression mg per job (mg/job) or mg/application refers to the amount of active substance used in relation to the total weight of the composition used for a complete cleaning cycle (which is to say in the case of detergent agents, the total amount of the cleaning agent used in a complete cleaning cycle of washing). In the case of preportioned cleaning agents, this information is the amount of the active substance in mg based on the total weight of the preportioned cleaning composition.

Said amounts are also applicable for each of the other individual enzyme proteins (e.g., amylase or protease) used in the dishwashing composition of the invention.

In some preferred embodiments, the detergent compositions provided herein are typically formulated such that, during use in aqueous cleaning operations, the wash water has a pH of from about 5.0 to about 13.5, or in alternative embodiments, even from about 6.0 to about 10.5, such as from about 5 to about 11, from about 5 to about 10, from about 5 to about 9, from about 5 to about 8, from about 5 to about 7, from about 6 to about 11, from about 6 to about 10, from about 6 to about 9, from about 6 to about 8, from about 6 to about 7, from about 7 to about 11, from about 7 to about 10, from about 7 to about 9, or from about 7 to about 8. Preferably, the detergent compositions provided herein are typically formulated such that, during use in aqueous cleaning operations, the wash water has a pH selected in the range from about 7.5 to about 13.5, further preferably said pH is selected in the range from about 8.5 to about 11.5, most preferably said pH is selected in the range from about 9.5 to about 10.5; further most preferably pH 7.5 or above.

In one embodiment, the beta-glucanase of the invention has improved stability, in particular improved storage stability in a high pH liquid cleaning composition, compared to known beta-glucanases. In a preferred embodiment, the beta-glucanase of the invention has improved stability, in particular improved storage stability, and on par or improved wash performance compared to the known beta-glucanases.

In some preferred embodiments, granular or liquid laundry products are formulated such that the wash water has a pH from about 5.5 to about 8. In other preferred embodiments, granular or liquid laundry products are formulated such that the wash water has a pH selected in the range from about 7.5 to about 13.5, further preferably said pH is selected in the range from about 8.5 to about 11.5, most preferably said pH is selected in the range from about 9.5 to about 10.5; further most preferably pH 7.5 or above. Techniques for controlling pH at recommended usage levels include the use of buffers, alkalis, acids, etc., and are well known to those skilled in the art.

Enzyme components weights are based on total protein. All percentages and ratios are calculated by weight unless otherwise indicated. All percentages and ratios are calculated based on the total composition unless otherwise indicated. In the exemplified detergent composition, the enzymes levels are expressed by pure enzyme by weight of the total composition and unless otherwise specified, the detergent ingredients are expressed by weight of the total composition.

The enzymes of the present invention also find use in detergent additive products. A detergent additive product comprising a beta-glucanase of the invention is suited for inclusion in a wash process when, e.g., temperature is low, such as at temperatures about 40° C. or below, the pH is between 6 and 8 and the washing time short, e.g., below 30 min. A detergent additive product comprising a beta-glucanase of the invention is further ideally suited for inclusion in an alkaline wash process when, e.g., a pH selected in the range from about 7.5 to about 13.5, a temperature selected in the range from about 20° C. to about 75° C., and the washing time short, e.g., below 30 min, e.g., at least 15 minutes. Alternatively, a detergent additive product comprising a beta-glucanase of the invention is suited for cleaning of a household dishwasher, e.g., from built-up residues on the filter and in the sump of the machines, preferably from residues containing beta-glucan-containing fibres. Such a machine-cleaning additive product may be suitable to clean at the same time from other residues like fat or limescale.

The detergent additive product may be a beta-glucanase of the invention and preferably an additional enzyme. In one embodiment, the additive is packaged in dosage form for addition to a cleaning process. The single dosage may comprise a pill, tablet, gelcap or other single dosage unit including powders and/or liquids. In some embodiments, filler and/or carrier material(s) are included, suitable filler or carrier materials include, but are not limited to, various salts of sulfate, carbonate and silicate as well as talc, clay and the like. In some embodiments filler and/or carrier materials for liquid compositions include water and/or low molecular weight primary and secondary alcohols including polyols and diols. Examples of such alcohols include, but are not limited to, methanol, ethanol, propanol and isopropanol.

In one particularly preferred embodiment the beta-glucanase according to the invention is employed in a granular composition or liquid, the beta-glucanase may be in form of an encapsulated particle. In one embodiment, the encapsulating material is selected from the group consisting of carbohydrates, natural or synthetic gums, chitin and chitosan, cellulose and cellulose derivatives, silicates, phosphates, borates, polyvinyl alcohol, polyethylene glycol, paraffin waxes and combinations thereof.

The compositions according to the invention typically comprise one or more detergent ingredients. The term detergent compositions include articles and cleaning and treatment compositions. The term cleaning composition includes, unless otherwise indicated, tablet, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, gel-form, liquid and rinse-aid types for household and institutional use.

The composition can also be in unit dose packages, including those known in the art and those that are water soluble, water insoluble and/or water permeable. These may encompass singlechamber and multichamber pouches.

In embodiments in which cleaning and/or detergent components may not be compatible with the beta-glucanase of the present invention, suitable methods may be used for keeping the cleaning and/or detergent components and the beta-glucanase separated (i.e., not in contact with each other) until combination of the two components is appropriate. Such separation methods include any suitable method known in the art (e.g., gelcaps, encapsulation, tablets, and physical separation e.g., by use of a water dissolvable pouch having one or more compartments).

As mentioned when the beta-glucanase of the invention is employed as a component of a detergent composition (e.g., a laundry washing detergent composition, or a dishwashing detergent composition), it may, for example, be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 (both to Novo Industri A/S) and may optionally be coated by methods known in the art. Examples of waxy coating materials are polyethyleneglycol (PEG) products with mean molecular weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). The enzymes of the detergent compositions of the invention may also be stabilized using conventional stabilizing agents such as polyol, e.g., propylene glycol or glycerol, a sugar or sugar alcohol, lactic acid, and the composition may be formulated as described in, e.g., WO 92/19709 and WO 92/19708. The enzymes of the invention may also be stabilized by adding reversible enzyme inhibitors, e.g., of the protein type (as described in EP 544777) or the boronic acid type. In a preferred embodiment the enzyme stabilizers are of the boronic acid type, more preferably 4-formyl phenyl boronic acid. The dishwashing composition of the invention is preferably free of boric acid and/or borate, which is to say in particular comprises boric acid and borate in amounts of less than 0.1 wt. %, preferably less than 0.01 wt. %, based on the total composition.

Other enzyme stabilizers are well known in the art, such as peptide aldehydes and protein hydrolysate, e.g., the beta-glucanase according to the invention may be stabilized using peptide aldehydes or ketones such as described in WO 2005/105826 and WO 2009/118375.

Protected enzymes for inclusion in a detergent composition of the invention may be prepared, as mentioned above, according to the method disclosed in EP 238 216.

The composition may be augmented with one or more agents for preventing or removing the formation of the biofilm. These agents may include, but are not limited to, dispersants, surfactants, detergents, other enzymes, anti-microbials, and biocides.

The compositions of the invention may be applied in dosing elements to be used in an auto-dosing device. The dosing elements comprising the composition of the present invention can be placed into a delivery cartridge as that described in WO 2007/052004 and WO 2007/0833141 or WO 2011/051420, WO 2011/051415, WO 2011/051416, WO 2011/051417, WO 2011/051418, WO 2011/120546 and WO 2011/131260. The dosing elements can have an elongated shape and set into an array forming a delivery cartridge which is the refill for an auto-dosing dispensing device as described in case WO 2007/051989. The delivery cartridge is to be placed in an auto-dosing delivery device, such as that described in WO 2008/053191.

Suitable disclosure of auto-dosing devices can be found in WO 2007/083139, WO 2007/051989, WO 2007/083141, WO 2007/083142 and EP2361964.

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to an ADW (Automatic Dish Wash) compositions comprising an enzyme of the present invention in combination with one or more additional ADW composition components. The choice of additional components is within the skill of the artisan and includes conventional ingredients, including the exemplary non-limiting components set forth below.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 2% to 60%, or about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

Non-limiting examples of zwitterionic surfactants include betaines such as alkyldimethylbetaines, sulfobetaines, and combinations thereof.

In one embodiment, the surfactant is a non-naturally occurring surfactant.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see, e.g., review by Hodgdon and Kaler, 2007, Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

In one embodiment, the builder or co-builder is a non-naturally occurring builder or co-builder.

### Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO 98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

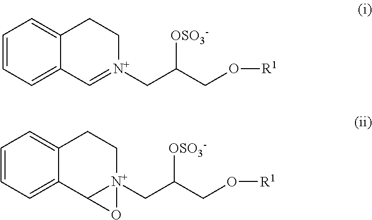

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g., in WO 2007/087258, WO 2007/087244, WO 2007/087259, EP 1867708 (Vitamin K) and WO 2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

In one embodiment, the bleaching system is a non-naturally occurring bleaching system.

### Polymers

The detergent may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(ethyleneglycol) or poly(ethylene oxide) (PEG or PEO), ethoxylated poly(ethyleneimine), (carboxymethyl)inulin (CMI), carboxylate polymers and polycarboxylates such as polyacrylates, maleic/acrylic acid copolymers, acrylate/styrene copolymers, poly(aspartic) acid, and lauryl methacrylate/acrylic acid copolymers, hydrophobically modified CMC (HM-CMC), silicones, copolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Adjunct Materials

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of:
(i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562. Further polymers useful in detergent formulations are described in WO 2007/149806.

In another aspect the detergent is a compact granular (powdered) detergent comprising a) at least about 10%, preferably from 15 to 60% by weight of the composition, of surfactant selected from anionic surfactants, non-ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

The soils and stains that are important for detergent formulators are composed of many different substances, and a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. Stain removing enzymes that are known in the art include enzymes such as carbohydrases, amylases, proteases, lipases, cellulases, hemicellulases, xylanases, cutinases, and pectinase.

Rheology Modifiers—The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Uses

The beta-glucanases of the invention may be used in applications where beta-glucan (e.g., on laminarin, lichenin and cereal beta-D-glucans, but not on substrates containing only 1,4-bonds) needs to be degraded (e.g., under alkaline conditions). Examples of where beta-glucanases could be used include detergent applications, paper and pulp productions. In one aspect, beta-glucanases of the invention may be used for washing or cleaning a textile and/or a hard surface such as dish wash including Automatic Dish Wash (ADW), Hand Dish Wash (HDW), and/or in a cleaning process such as laundry or hard surface cleaning including dish wash including Automatic Dish Wash (ADW) and industrial cleaning, and/or for laundering and/or hard surface cleaning including dish wash including Automatic Dish Wash (ADW), and/or for at least one of the following: preventing, reducing or removing a biofilm and/or malodor from an item, and/or for anti-redeposition.

Such beta-glucanases preferably have at least 60%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of the sequence selected from the group consisting of: SEQ ID NO: 3, SEQ ID NO: 6, SEQ ID NO: 9, SEQ ID NO: 12, SEQ ID NO: 15, SEQ ID NO: 18.

The present invention concerns the use of the beta-glucanases for soil and/or stain removal. Contemplated are removal of cereal containing soil, especially dried-on cereal containing soil, preferably oat flakes containing soil, especially dried-on oat flakes containing soil and/or cooked oats containing soil, and/or cooked and burned-in oats containing soil, and/or uncooked oats containing soil, removal of chocolate containing soil, especially chocolate porridge oats containing soil, and/or chocolate milkshake containing soil, and/or chocolate drinks containing soil; removal of cosmetics and/or personal care containing soil; removal of tomato containing soil, especially tomato soup containing soil, and/or tomato sauce, such as spaghetti sauce containing soil; facilitating removal of starch-containing soil in the presence of one or more amylases and/or for enhancing amylase related cleaning performance; facilitating removal of protein-containing soil in the presence of one or more proteases and/or for enhancing protease related cleaning performance; and/or facilitating removal of carbohydrase-containing soil in the presence of one or more other carbohydrases and/or enhancing carbohydrase related cleaning performance. Biofilm can develop on textile when microorganisms are present on an item and stick together on the item. Some microorganisms tend to adhere to the surface of items such as textiles. Some microorganisms adhere to such surfaces and form a biofilm on the surface. The biofilm may be sticky and the adhered microorganisms and/or the biofilm may be difficult to remove. Furthermore, the biofilm adhere soil due to the sticky nature of the biofilm. The commercial laundry detergent compositions available on the market do not remove such adhered microorganisms or biofilm.

The present invention concerns the use of a polypeptide(s) having beta-glucanase activity for preventing, reducing or removing a biofilm from an item, wherein the polypeptide is obtained from a bacterial source and wherein the item is a textile. In one embodiment of the invention the polypeptide having beta-glucanase activity is used for preventing, reducing or removing the stickiness of an item.

In an embodiment is provided use of a polypeptide(s) having beta-glucanase activity for cleaning, e.g., deep cleaning of an item, wherein the item is a textile or a surface.

### Washing Method

The detergent compositions of the present invention are ideally suited for use in laundry applications. Accordingly, the present invention includes a method for laundering a fabric. The method comprises the steps of contacting a fabric to be laundered with a cleaning laundry solution comprising the detergent composition according to the invention. The fabric may comprise any fabric capable of being laundered in normal consumer use conditions. The solution preferably has a pH of from about 5.5 to about 8, further preferably pH selected in the range from about 7.5 to about 13.5, or in the range from about 7.5 to about 12.5, or in the range from about 8.5 to about 11.5, or in the range from about 9.5 to about 10.5, or pH 7.5 or above.

A preferred embodiment concerns a method of cleaning, the method comprising the steps of: contacting an object with a high pH cleaning composition (e.g., pH 7.5 or above) comprising a beta-glucanase of the invention under conditions suitable for cleaning the object. In a preferred embodiment the cleaning composition is used in a laundry or a dish wash process.

Still another embodiment relates to a method for removing stains from fabric or dishware which comprises contacting the fabric or dishware with a high pH cleaning composition (e.g., pH 7.5 or above) comprising a beta-glucanase of the invention under conditions suitable for cleaning the object.

Also contemplated are compositions and methods of treating fabrics (e.g., to desize a textile) using the cleaning composition of the invention. The high pH cleaning composition can be used in any fabric-treating method which is well known in the art.

In another embodiment the high pH cleaning composition of the present invention is suited for use in liquid laundry and liquid hard surface applications, including dish wash and car wash. Accordingly, the present invention includes a method for laundering a fabric or washing a hard surface. The method comprises the steps of contacting the fabric/dishware to be cleaned with a solution comprising the high pH cleaning composition according to the invention. The fabric may comprise any fabric capable of being laundered in normal consumer use conditions. The hard surface may comprise any dishware such as crockery, cutlery, ceramics, plastics such as melamine, metals, china, glass, acrylics or other hard surfaces such as cars, floors etc. The solution preferably has a pH, e.g., 7.5 or above, e.g., from about 9 to about 13.5.

The compositions may be employed at concentrations of from about 100 ppm, preferably 500 ppm to about 15,000 ppm in solution. The water temperatures typically range from about 5° C. to about 90° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8. In preferred embodiments the washing method is conducted at a pH selected in the range from about 7.5 to about 13.5, or in the range from about 7.5 to about 12.5, or in the range from about 8.5 to about 11.5, or in the range from about 9.5 to about 10.5, or pH 7.5 or above.

In some preferred embodiments, the high pH cleaning compositions provided herein are typically formulated such that, during use in aqueous cleaning operations, the wash water has a pH of from about 9 to about 13.5, or in alternative embodiments, or from about 10 to about 13.5 even from about 11 to about 13.5. In some preferred embodiments the liquid laundry products are formulated to have a pH from about 12 to about 13.5. Techniques for controlling pH at recommended usage levels include the use of buffers, acids, alkalis, etc., and are well known to those skilled in the art. In the context of the present invention alkalis are used to adjust pH to about 9 to 13.5 preferably about 10 to 13.5.

In particular embodiments, the washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

The present invention relates to a method of cleaning a fabric, a dishware or hard surface with a detergent composition comprising a beta-glucanase of the invention.

A preferred embodiment concerns a method of cleaning, said method comprising the steps of: contacting an object with a cleaning composition comprising a beta-glucanase of the invention under conditions suitable for cleaning said object. In a preferred embodiment the cleaning composition is a detergent composition and the process is a laundry or a dish wash process.

Still another embodiment relates to a method for removing stains from fabric which comprises contacting said a fabric with a composition comprising a beta-glucanase of the invention under conditions suitable for cleaning said object.

### Low Temperature Uses

One embodiment of the invention concerns a method of doing laundry, dish wash or industrial cleaning comprising contacting a surface to be cleaned with a beta-glucanase of the invention, and wherein said laundry, dish wash, industrial or institutional cleaning is performed at a temperature of about 40° C. or below. One embodiment of the invention relates to the use of a beta-glucanase in laundry, dish wash or a cleaning process wherein the temperature in laundry, dish wash, industrial cleaning is about 40° C. or below.

In another embodiment, the invention concerns the use of a beta-glucanase according to the invention in a beta-glucan removing process, wherein the temperature in the beta-glucan removing process is about 40° C. or below.

In each of the above-identified methods and uses, the wash temperature is about 40° C. or below, such as about 39° C. or below, such as about 38° C. or below, such as about 37° C. or below, such as about 36° C. or below, such as about 35° C. or below, such as about 34° C. or below, such as about 33° C. or below, such as about 32° C. or below, such as about 31° C. or below, such as about 30° C. or below, such as about 29° C. or below, such as about 28° C. or below, such as about 27° C. or below, such as about 26° C. or below, such as about 25° C. or below, such as about 24° C. or below, such as about 23° C. or below, such as about 22° C. or below, such as about 21° C. or below, such as about 20° C. or below, such as about 19° C. or below, such as about 18° C. or below, such as about 17° C. or below, such as about 16° C. or below, such as about 15° C. or below, such as about 14° C. or below, such as about 13° C. or below, such as about 12° C. or below, such as about 11° C. or below, such as about 10° C. or below, such as about 9° C. or below, such as about 8° C. or below, such as about 7° C. or below, such as about 6° C. or below, such as about 5° C. or below, such as about 4° C. or below, such as about 3° C. or below, such as about 2° C. or below, such as about 1° C. or below.

In another preferred embodiment, the wash temperature is in the range of about 5-40° C., such as about 5-30° C., about 5-20° C., about 5-10° C., about 10-40° C., about 10-30° C., about 10-20° C., about 15-40° C., about 15-30° C., about 15-20° C., about 20-40° C., about 20-30° C., about 25-40° C., about 25-30° C., or about 30-40° C. In particular preferred embodiments the wash temperature is about 20° C., about 30° C., or about 40° C.

### High Temperature Uses

One embodiment of the invention concerns a method of doing laundry, dish wash or industrial cleaning comprising contacting a surface to be cleaned with a beta-glucanase of the invention, and wherein said laundry, dish wash, industrial or institutional cleaning is performed at a temperature of about 75° C. or below. One embodiment of the invention relates to the use of a beta-glucanase in laundry, dish wash or a cleaning process wherein the temperature in laundry, dish wash, industrial cleaning is about 70° C. or below.

In another embodiment, the invention concerns the use of a beta-glucanase according to the invention in a beta-glucan removing process, wherein the temperature in the beta-glucan removing process is about 65° C. or below.

In each of the above-identified methods and uses, the wash temperature is about 60° C. or below, such as about 59° C. or below, such as about 58° C. or below, such as about 57° C. or below, such as about 56° C. or below, such as about 55° C. or below, such as about 54° C. or below, such as about 53° C. or below, such as about 52° C. or below, such as about 51° C. or below, such as about 50° C. or below, such as about 49° C. or below, such as about 48° C. or below, such as about 47° C. or below, such as about 46° C. or below, such as about 45° C. or below, such as about 44° C. or below, such as about 43° C. or below, such as about 42° C. or below, such as about 41° C. or below.

In another preferred embodiment, the wash temperature is in the range of about 41-90° C., such as about 41-80° C., about 41-85° C., about 41-80° C., about 41-75° C., about 41-70° C., about 41-65° C., about 41-60° C.

An embodiment of the invention is a method for reducing or preventing soil redeposition using a detergent composition comprising a polypeptide(s) of the invention.

In one embodiment, the detergent composition further comprises one or more detergent components selected from the group comprising surfactants, builders, hydrotopes, bleaching systems, polymers, fabric hueing agents, adjunct materials, dispersants, dye transfer inhibiting agents, fluorescent whitening agents and soil release polymers, or any mixture thereof. The detergent composition may be in the form of a bar, a homogenous tablet, a tablet having two or more layers, a pouch having one or more compartments, the compartment(s) containing one or more different phases, a regular or compact powder, a granulate, a paste, a gel, a spray, or a regular, compact or concentrated liquid, two or more liquids and/or gels in a multichamber-bottle and may be used for dish wash or laundering.

In another embodiment, the detergent composition comprises one or more additional enzymes selected from the group comprising proteases, amylases, lipases, cutinases, cellulases, endoglucanases, xyloglucanases, pectinases, pectin lyases, xanthanases, peroxidaes, haloperoxygenases, catalases, mannanases, licheninases, phosphodiesterases, or any mixture thereof.

In a further embodiment, the detergent composition comprises one or more detergent components selected from the group comprising surfactants, builders, hydrotopes, bleaching systems, polymers, fabric hueing agents, adjunct materials, dispersants, dye transfer inhibiting agents, fluorescent whitening agents and soil release polymers, or any mixture thereof and one or more additional enzymes selected from the group comprising proteases, amylases, lipases, cutinases, cellulases, endoglucanases, xyloglucanases, pectinases, pectin lyases, xanthanases, peroxidaes, haloperoxygenases, catalases, mannanases, licheninases, phosphodiesterases, or any mixture thereof.

The method may comprise the following steps:
(a) providing a wash liquor by dissolving/mixing the detergent composition in water;
(b) washing the objects/fabrics/textiles in the wash liquor;
(c) draining the wash liquor and optionally repeating the wash cycle; and
(d) rinsing and optionally drying the objects/fabrics/textiles.

In a preferred embodiment the method may comprise the following steps:
(1) providing water and rinsing the objects
(2) optionally, draining the water and providing fresh water
(3) dosing the detergent composition into the water to form a wash liquor
(4) agitating the wash liquor, thereby washing the objects, optionally heating the liquor
(5) draining the wash liquor
(6) optionally providing fresh water, rinsing the objects, and draining the liquid
(7) optionally providing fresh water, rinsing the objects, and during this step dosing an optional additional agent into the liquor, e.g., a rinse-aid, optionally heating the liquor, and afterwards draining the liquor.
(8) optionally letting remaining liquid evaporate from the objects.

A preferred embodiment of the invention is a method for reducing soil redeposition using a detergent composition comprising a polypeptide having at least 60% sequence identity to the polypeptide of the sequence selected from the group consisting of: SEQ ID NO: 3, SEQ ID NO: 6, SEQ ID NO: 9, SEQ ID NO: 12, SEQ ID NO: 15, and SEQ ID NO: 18.

A preferred embodiment of the invention is a method for reducing soil redeposition using a detergent composition comprising: a polypeptide(s) having beta-glucanase activity, selected from the group consisting of: SEQ ID NO: 3, SEQ ID NO: 6, SEQ ID NO: 9, SEQ ID NO: 12, SEQ ID NO: 15, and SEQ ID NO: 18, wherein said cleaning or detergent composition further comprises:
(i) one or more amylases; and/or
(ii) one or more proteases.

A preferred embodiment of the invention is a method for removing soils, and/or facility removal of soils, using a detergent composition comprising a polypeptide having at least 60% sequence identity to the polypeptide of the sequence selected from the group consisting of: SEQ ID NO: 3, SEQ ID NO: 6, SEQ ID NO: 9, SEQ ID NO: 12, SEQ ID NO: 15, and SEQ ID NO: 18.

The present invention is further described by the following examples that should not be construed as limiting the scope of the invention.

### EXAMPLES

Detergent compositions used in the example sections as described herein included the following:

TABLE A
Model detergent A:

| | Content of | Active |
|---|---|---|
| | compound | component |
| Compound | (% w/w) | (% w/w) |
| LAS | 12.0 | 97 |
| AEOS, SLES | 17.6 | 28 |
| Soy fatty acid | 2.8 | 90 |
| Coco fatty acid | 2.8 | 99 |
| AEO | 11.0 | 100 |
| Sodium hydroxide | 1.8 | 99 |
| Ethanol/Propan-2-ol | 3.0 | 90/10 |
| MPG | 6.0 | 98 |
| Glycerol | 1.7 | 99.5 |
| TEA | 3.3 | 100 |
| Sodium formate | 1.0 | 95 |
| Sodium citrate | 2.0 | 100 |
| DTMPA (as Na7-salt) | 0.5 | 42 |
| PCA (as Na-salt) | 0.5 | 40 |
| Phenoxy ethanol | 0.5 | 99 |
| Ion exchanged water | 33.6 | — |

Water hardness was adjusted to 15° dH by addition of CaCl₂, MgCl₂, and NaHCO₃ (Ca²⁺:Mg²⁺:HCO₃₋ = 4:1:7.5) to the test system.

TABLE B
Model detergent X:

| | Content | Active |
|---|---|---|
| | of compound | component |
| Compound | (% w/w) | (% w/w) |
| LAS | 16.5 | 91 |
| AEO* | 2 | 99.5 |
| Sodium carbonate | 20 | 100 |
| Sodium (di)silicate | 12 | 82.5 |
| Zeolite A | 15 | 80 |
| Sodium sulfate | 33.5 | 100 |
| PCA | 1 | 100 |

*Model detergent X was mixed without AEO. AEO was added separately before wash.
Water hardness was adjusted to 12° dH by addition of CaCl₂, MgCl₂, and NaHCO₃ (Ca²⁺:Mg²⁺:HCO₃₋ = 2:1:4.5) to the test system.

TABLE C
ADW model detergent A:

| | Content | Active |
|---|---|---|
| | of compound | component |
| Compound | (% w/w) | (% w/w) |
| MGDA (Trilon | 20 | 59 |
| M Granules SG) | | |
| Sodium citrate | 20 | 100 |
| Sodium carbonate | 20 | 100 |
| Sodium percarbonate | 10 | 88 |
| Sodium silicate | 5 | 80 |
| Sodium sulfate | 12 | 100 |
| Acusol 588G | 5 | 92 |
| TAED | 3 | 92 |
| Surfac 23-6.5 (liq) | 5 | 100 |

Water hardness was adjusted to 21° dH by addition of CaCl₂, MgCl₂, and NaHCO₃ (Ca²⁺:Mg²⁺:HCO₃₋ = 4:1:10) to the test system.

### Wash Performance

### Automatic Mechanical Stress Assay (AMSA)

The wash performance in laundry washing is assessed using the Automatic Mechanical Stress Assay (AMSA). With the AMSA, the wash performance of a large quantity of small volume enzyme-detergent solutions can be examined. The AMSA plate has a number of slots for test solutions and a lid firmly squeezing the laundry sample, the textile to be washed against all the slot openings. During the washing time, the plate, test solutions, textile and lid are vigorously shaken to bring the test solution in contact with the textile and apply mechanical stress in a regular, periodic oscillating manner.

The wash performance is measured as the brightness of the colour of the textile washed. Brightness can also be expressed as the intensity of the light reflected from the sample when illuminated with white light. When the sample is stained the intensity of the reflected light is lower, than that of a clean sample. Therefore, the intensity of the reflected light can be used to measure wash performance.

Colour measurements are made with a professional flatbed scanner (Kodak iQsmart, Kodak, Midtager 29, DK-2605 Brondby, Denmark), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

$$Int = \sqrt{{r}^{2} + {g}^{2} + {b}^{2}}$$

The experiments are conducted as described in the Automatic Mechanical Stress Assay (AMSA) for laundry method using a 1 cycle wash procedure and the experimental conditions specified in Table A.

TABLE A
Conditions for AMSA Washing Trial

| Test Solution | liquid detergent 1 g/L |
|---|---|
| | powder detergent 1.6 g/L |
| | or as otherwise noted |
| Test solution volume | 160 μL |
| pH | pH unadjusted |
| Wash time | 20 minutes |
| Temperature | 20° C. or 40° C. |
| Water hardness | 15° dH |
| Ca²⁺:Mg²⁺:CO₃²⁻ ratio | 4:1:7.5 |

Water hardness is adjusted by addition of CaCl₂, MgCl₂, and NaHCO₃ to the test system. After washing the textiles are flushed in tap water and air-dried.

### Terg-O-Tometer (TOM) Washing Trial

The terg-o-tometer is an industry standard. 1 L of wash solution is incubated in a water bath temperature controlled environment. The solution is mixed for 10 min before adding 1 L to each of the beakers. The temperature in the beakers is measured to be 20.0° C. The washed and rinsed swatches are left to dry overnight in a drying cabinet.

TABLE B
Conditions for Terg-O-tometer Washing Trial

| | |
|---|---|
| Detergent dosage | 3.33 g/L or as noted |
| Test solution volume | 1 L |
| pH | unadjusted |
| Wash time | 30 minutes or as noted |
| Temperature | 20° C. or as noted |
| Water hardness | 15° dH |
| Ca²⁺:Mg²⁺:CO₃²⁻ ratio | 4:1:7.5 |
| Mechanical action | 120 rpm |
| Enzyme dose | 0.001-0.05 ppm or as noted |

After washing and rinsing the swatches were spread out flat and allowed to air dry at room temperature overnight. Wash performance is expressed as a delta remission value (ΔRem). Light reflectance evaluations of the swatches are done using a Macbeth Color Eye 7000 reflectance spectrophotometer with large aperture. The measurements are made without UV in the incident light and remission at 460 nm was extracted. The dry swatches are measured with ColorEye 2. Measurement with small aperture through 2 layers (2 of the same type of swatch from the same beaker), 1 measurements on each swatch on the front side marked with beaker and swatch number. Remission values for individual swatches are calculated by subtracting the remission value of the control swatch from the remission value of the washed swatch. Calculating the enzyme effect is done by taking the measurements from washed swatches with enzymes and subtract with the measurements from washed without enzyme for each stain. The total enzyme performance is calculated as the average of individual ΔRem_{enzyme}.

### Launder-O-Meter (LOM) Model Wash System

The Launder-O-Meter (LOM) is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM or is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Example 1: Assays

### Assay I: Determination of Beta-Glucanase (Laminarinase) Activity:

AZCL-curdlan and AZCL-pachyman (azurine dye covalently cross-linked beta-glucans from Megazyme) assay are used for detection of endo-glucanase activity (laminarinase activity).

AZCL-curdlan or AZCL-pachyman (75 mg) was suspended in 15 mL detergent (Model detergents A, X, or ADW Model A). To 1 mL of this solution in Eppendorf tubes was added 100 μL enzyme (0.033 mg enzyme protein per millilLiter), incubated for 15 min at 40° C. while shaking at 1250 rpm in a pre-heated thermos-mixer and spun down for 2 min at 13200 rpm, diluted 5 or 10 times with a 5% Triton-X-100 including 10 μM CaCl₂ and 250 μL of the solution was transferred to a micro-titer plate and the sample absorbance was measured at 590 nm.

### Assay II: Testing of Alpha-Amylase Activity

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473). The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0). The alpha-Glucosidase reagent contains 52.4 mM HEPES, 87 mM NaCl, 12.6 mM MgCl₂, 0.075 mM CaCl₂, ≥4 kU/L alpha-glucosidase). The substrate working solution is made by mixing 1 mL of the alpha-Glucosidase reagent with 0.2 mL of the G7-pNP substrate. This substrate working solution is made immediately before use. Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl2, pH8.0. The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm. The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Assay III: Testing of Cellulase Activity

An AZCL-He-cellulose (azurine dye covalently cross-linked cellulose) assay is used for detection of cellulase (endo-glucanase) activity. AZCL-He-cellulose (75 mg) is suspended in 15 mL detergent (e.g., Model detergent A). To 1 mL of this solution in Eppendorf tubes is added 100 μL enzyme (0.09 mg enzyme protein/mL), incubated for 15 min at 40° C. while shaking at 1250 rpm in a pre-heated thermo mixer and spun down for 2 min at 13200 rpm. 250 μL of the solution is transferred to a micro-titer plate and the sample absorbance is measured at 590 nm.

### Assay IV: Testing of DNase Activity

DNase activity is determined on DNase Test Agar with Methyl Green (BD, Franklin Lakes, NJ, USA), which is prepared according to the manual from supplier. Briefly, 21 g of agar is dissolved in 500 ml water and then autoclaved for 15 min at 121° C. Autoclaved agar is temperated to 48° C. in water bath, and 20 ml of agar is poured into petri dishes with and allowed to solidify by incubation o/n at room temperature. On solidified agar plates, 5 μl of enzyme solutions are added and DNase activity is observed as colorless zones around the spotted enzyme solutions.

### Assay V: Testing of DNase Activity

DNase activity is determined by using the DNaseAlert™ Kit (11-02-01-04, IDT Intergrated DNA Technologies) according to the supplier's manual. Briefly, 95 μl DNase sample is mixed with 5 μl substrate in a microtiter plate, and fluorescence is immediately measured using a Clariostar microtiter reader from BMG Labtech (536 nm excitation, 556 nm emission).

### Assay VI: Testing of Lipase Activity

Lipase is diluted with a buffer (10 mM Succinic acid+2 mM CaCl₂+0.02% Brij 35 adjusted to Ph 6.5) to the specified concentration. 10 uL of the 100 ppm lipase solution is added to a 90 uL of detergent composition, stirred for 5 minutes and sealed. Samples are stored at 4° C. in detergent D002 (unstressed) and in detergent D002 at 47° C. (stressed). Storage time is 335.5 hours. After storage possible condensation liquid is collected by centrifugation. To the 100 uL stressed or unstressed sample 235 uL of buffer (0.1 M Tris-HCl, 9 mM CaCl2, 0.0225% Brij-30, pH 8.0+0.85% 4-FBPA (31.5 g/I)) are added corresponding to a 3.35-fold dilution. After 10 minutes stirring 5 uL sample aliquots are further diluted with the same buffer 60-fold. Then one part of this lipase dilution is mixed with four parts of 0.5 mM pNP-palmitate, 1 mM calcium chloride, 100 mM Tris (pH 8.0), 6.5 mM Deoxycholate, 1.4 g/L AOS and for 30 minutes release of the pNP chromophore is measured spectrophotometrically. This is used to determine activity via the initial linear slope of the reaction. Residual activity is calculated as the ratio of the measured velocities of stressed versus unstressed sample. The median value of the residual activity is calculated based on four replicates and normalized by a lipase variant reference run with each experimental set.

### Assay VII: Testing of Mannanase Activity

Mannanase activity may be tested according to standard test procedures known in the art, such as by applying a solution to be tested to 4 mm diameter holes punched out in agar plates containing 0.2% AZCL galactomannan (carob), i.e., substrate for the assay of endo-1,4-beta-D-mannanase available as CatNo.I-AZGMA from the company Megazyme (available on the Internet at megazyme.com/purchase/index.html).

### Assay VIII: Testing of Pectinase Activity

### Microtiter Assay for Quantification of Pectate Lyase Activity

Pectate lyase cleaves polygalacturonic acid through a trans elimination mechanism. This means that it leaves a double C—C bond for each substrate split. This bond absorbs at 235 nm allowing direct detection of pectate lyase action on soluble polygalacturonic acid by measuring absorbance at that wavelength.

An enzyme sample is diluted in assay buffer (100 mM Tris-HCl, 0.68 mM CaCl₂, pH 8.0) to a concentration between 5 and 100 ng/ml. If the enzyme sample contains detergent it should be diluted at least a 1000-fold with respect to detergent. 100 μl of the enzyme buffer dilution is mixed with 100 μl substrate (1% (w/v) polygalacturonic acid, e.g., P-3850 from Sigma, stirred in assay buffer for at least 15 min and centrifuged for 5 min at 2300 g, supernatant is used) in a heating plate and heated to 40° C. for 10 min in a heating block, preferably a PCR machine or equipment of equivalent accuracy and heating speeds.

100 μl enzyme/substrate solution is mixed with 100 μl stop reagent (50 mM H₃PO₄) in a UV-transparent microtiter plate. The UV plate is shaken briefly and gently, and the absorbance at 235 nm is measured in a microtiter spectrometer (e.g., Molecular Devices, SpectraMAX 190). The absorbance readings are corrected for background absorbance by subtracting the absorbance of a control sample, run without enzyme added, to all measured values.

Alternatively, catalytic activity of pectate lyase can be determined by the viscosity assay, APSU.

### Viscosity Assay, APSU

APSU units: The APSU assay measures the change in viscosity of a solution of polygalacturonic acid in the absence of added calcium ions. A 5% w/v solution of sodium polygalacturonate (e.g., Sigma P-1879) is solubilised in 0.1 M glycine buffer, pH 10. 4 ml of this solution are preincubated for 5 min at 40° C. Then, 250 microlitre of the enzyme (or enzyme dilution) are added, after which the reaction is mixed for 10 sec on a mixer at the highest speed and incubated for 20 min at 40° C. or at another temperature.

### Assay IX: Testing of Protease Activity

Proteolytic activity can be determined by a method employing Suc-AAPF-PNA as the substrate. Suc-AAPF-PNA is an abbreviation for N-Succinyl-Alanine-Alanine-Proline-Phenylalanine-p-Nitroanilide and is a blocked peptide which can be cleaved by endo-proteases. Following cleavage, a free PNA molecule is liberated, which has a yellow color and thus can be measured by visible spectrophotometry at wavelength 405 nm. The Suc-AAPF-PNA substrate is manufactured by Bachem (cat. no. L1400, dissolved in DMSO). The protease sample to be analyzed is diluted in residual activity buffer (100 mM Tris pH 8.6). The assay is performed by transferring 3 0 μl of diluted enzyme samples to 96 well microtiter plate and adding 70 μl substrate working solution (0.72 mg/ml in 100 mM Tris pH 8.6). The solution is mixed at room temperature and absorption is measured every 20 seconds over 5 minutes at OD 405 nm. The slope (absorbance per minute) of the time dependent absorption-curve is directly proportional to the activity of the protease in question under the given set of conditions. The protease sample is diluted to a level where the slope is linear.

### Example 2: Cloning, Expression and Purification of Bacterial Beta-Glucanases

The beta-glucanases were derived from bacterial strains isolated from environmental soil samples by standard microbiological isolation techniques. The isolated pure strains were identified and taxonomy was assigned based on DNA sequencing of the 16S ribosomal gene (Table 1).

TABLE 1

| Strain | Source Country | Mature protein SEQ ID: |
|---|---|---|
| Thermobacillus sp. | Mexico | SEQ ID NO: 3 |
| Paenibacillus sp. | Sweden | SEQ ID NO: 6 |
| Cohnella sp. | Denmark | SEQ ID NO: 9 |
| Paenibacillus elgii | United States | SEQ ID NO: 12 |
| Bacillus species A | Australia | SEQ ID NO: 15 |
| Bacillus species B | Antarctica | SEQ ID NO: 18 |

Chromosomal DNA was isolated from pure culture with the DNeasy Blood & Tissue Kit from Qiagen (Hilden, Germany) and subjected to full genome sequencing using Illumina technology. Genome sequencing, the subsequent assembly of sequencing reads and the gene discovery (i.e., annotation of gene functions) is known to the person skilled in the art and the service can be purchased commercially.

The genome sequence was analyzed for putative beta-glucanases from the Carbohydrate Active Enzymes database (CAZY) family GH16 (Lombard V et al. 2014. Nucleic Acids Res 42:D490-D495.) and this analysis identified six genes encoding a putative beta-glucanase enzymes which were subsequently cloned and recombinantly expressed in Bacillus subtilis.

The genes encoding the beta-glucanases were amplified as single amplicons by PCR and fused with regulatory elements, an affinity purification tag and homology regions for recombination into the pectate lyase locus of the B. subtilis genome.

The linear integration construct was a SOE-PCR fusion product (Horton et al., 1989, Engineering hybrid genes without the use of restriction enzymes, gene splicing by overlap extension, Gene 77: 61-68) made by fusion of the gene between two B. subtilis chromosomal regions along with strong promoters and a chloramphenicol resistance marker. The SOE-PCR method is also described in WO 2003/095658.

The genes were expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence.

The genes were fused with DNA encoding a Bacillus clausii secretion signal (encoding the following amino acid sequence: MKKPLGKIVASTALLISVAFSSSIASA (SEQ ID NO: 19) replacing the native secretion signal. Furthermore, the expression construct results in the addition of a amino-terminal poly histidine tail consisting of the amino acid sequence HHHHHHPR(SEQ ID NO: 20) to the mature beta-glucanase.

The SOE-PCR product was transformed into Bacillus subtilis and integrated in the chromosome by homologous recombination into the pectate lyase locus. Subsequently a recombinant Bacillus subtilis clone containing the integrated expression construct was grown in liquid culture. The culture broth was centrifuged (20000×g, 20 min) and the supernatant was carefully decanted from the precipitate and used for purification of the enzyme or alternatively sterile filtered supernatant was used directly for assays.

### Purification of the Recombinant Enzymes by Nickel Affinity Chromatography

The pH of the cleared supernatant was adjusted to pH 8, filtrated through a 0.2 μM filter, and the supernatants applied to a 5 ml HisTrap™ excel column. Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Example 3: Construction of a Glycoside Hydrolase Family 16 Phylogenetic Tree and Identification of a Laminarinase Clade from the Order Bacillales

A phylogenetic tree was constructed with polypeptide sequences containing a GH16 domain, as defined in the CAZY database (Carbohydrate-active enzymes database (CAZy) in 2013. Nucleic Acids Research 42:D490-D495. Lombard et al.). The phylogenetic tree was constructed from a multiple alignment of mature polypeptide sequences containing at least one GH16 domain. The sequences were aligned using the MUSCLE algorithm version 3.8.31 (Edgar, 2004, Nucleic Acids Research 32(5): 1792-1797), and the trees were constructed using FastTree version 2.1.8 (Price et al., 2010, PloS one 5(3)) and visualized using iTOL (Letunic & Bork, 2007, Bioinformatics 23(1): 127-128).

The polypeptides in the GH16 glycoside hydrolase family can be separated into multiple distinct sub-clusters or clades when visualized as a phylogenetic tree and the clades can be observed to contain polypeptides encoding enzyme with different enzymatic specificities both within beta-glucan active enzymes and outside.

Mapping of the experimentally the verified laminarinase polypeptides express in Example 2 onto the phylogenetic tree allows the elucidation of a laminarinase specific clade of polypeptides and the determination of specific motifs associated with the clade.

Identification of a Laminarinase Clade from the Order Bacillales.

The clade comprises GH16 glycoside hydrolase family polypeptides of bacterial origin from the order Bacillales having laminarinase activity and comprising the conserved polypeptide motifs GEXDXME (SEQ ID NO: 28), GXGNXEXXXY (SEQ ID NO: 29), NXAXGG (SEQ ID NO: 30) and YTS[GA][KR] where X denotes any natural amino acid and residues in bracket [GA] means either G or A and [KR] means either K or R.

The 3D structure of a laminarinase from the bacteria Thermotoga maritima (SEQ ID NO: 27) has been experimentally determined. (Crystal structures of the laminarinase catalytic domain from Thermotoga maritima MSB8 in complex with inhibitors: essential residues for ß-1,3- and ß-1,4-glucan selection. Jeng et al., 2011, J Biol Chem. 286(52):45030-40).

Based on this structure and multiple alignments of the polypeptide sequences from SEQ ID NO: 27 and the experimental laminarinases from Example 2 one can on the basis of sequence homology deduce that the two glutamic acid(E) residues of the GEXDXME (SEQ ID NO: 28) are analogous to residues E132 and E137 of SEQ ID NO: 27 which are directly involved as catalytic residues and are essential for catalysis.

The asparagine(N) and glutamic acid(E) residues in the GXGNXEXXXY (SEQ ID NO: 29) motif are analogous to residue N45 and E47 in SEQ ID NO: 27 which may form direct or water-mediated hydrogen bonds to the laminarin substrate aiding in positioning the substrate correctly in the catalytic groove of the enzyme.

The asparagine residue (N) in the NXAXGG (SEQ ID NO: 30) motif is analogous to the N225 residue in SEQ ID NO: 27 which is suggested to have a role in substrate binding.

The arginine or lysine in the YTS[GA][KR] motif carry a positive charge and, without being bound by theory, this charge is proposed to be important for correct substrate interaction either through direct or water mediated interaction and is analogous to the R85 residue of the SEQ ID NO: 27.

### Example 4: AZCL-Assay with Beta-Glucanase Enzymes

In this example, enzymatic activity were measured on AZCL-curdlan and AZCl-pachyman substrate in detergents thus modeling various laundry, automatic dish wash and hand dish wash conditions. Measurements of enzymatic activity were carried out as described in Example 1, using the dilution factor as noted.

TABLE 2
Beta-glucanase Activity in Model Detergent A
Model detergent A (after 15 min at 40° C. incubation, pH 7.7)

| | | | Absorbance at 590 nm | |
|---|---|---|---|---|
| | Absorbance at 590 nm (Blank) | | (Laminarinase) | |
| Enzyme | AZCL-curdlan^{*2} | AZCL-pachyman | AZCL-curdlan^{*2} | AZCL-pachyman |
| Bacillus species B | 0.057 | 0.572 | 0.68 | 0.857 |
| Paenibacillus sp | 0.057 | 0.15^{*2} | 0.47 | 0.26^{*2} |
| Thermobacillus sp | 0.057 | 0.15^{*2} | 0.437 | 0.364^{*2} |
| Cohnella sp | 0.057 | 0.15^{*2} | 0.951 | 0.577^{*2} |
| Paenibacillus elgii | 0.057 | 0.091^{*2} | 1.707 | 0.775^{*2} |
| Bacillus species A | 0.050 | 0.139^{*1} | 0.913 | 0.261^{*1} |

^{*1}diluted 5 times
^{*2}diluted 10 times

TABLE 3
Beta-glucanase Activity in Model Detergent X
Model detergent X (after 15 min at 40° C. incubation, pH 10.1)

| | | | Absorbance at 590 nm | |
|---|---|---|---|---|
| | Absorbance at 590 nm (Blank) | | (Laminarinase) | |
| Enzyme | AZCL-curdlan | AZCL-pachyman | AZCL-curdlan | AZCL-pachyman |
| Bacillus species B | 0.082^{*2} | 0.67 | 0.416^{*2} | 0.876 |
| Paenibacillus sp | 0.163 | 0.67 | 0.231 | 0.764 |
| Thermobacillus sp | 0.082^{*2} | 0.369^{*2} | 0.125^{*2} | 0.556^{*2} |
| Cohnella sp | 0.163 | 0.67 | 0.182 | 0.745 |
| Paenibacillus elgii | 0.058^{*2} | 0.118^{*2} | 0.757^{*2} | 0.218^{*2} |
| Bacillus species A | 0.058^{*2} | 0.709 | 0.954^{*2} | 0.953 |

^{*2}diluted 10 times

TABLE 4
Beta-glucanase Activity in ADW Model detergent A
ADW Model detergent A (after 15 min at 40° C. incubation, pH 10.1)

| | | | Absorbance at 590 nm | |
|---|---|---|---|---|
| | Absorbance at 590 nm (Blank) | | (Laminarinase) | |
| Enzyme | AZCL-curdlan | AZCL-pachyman | AZCL-curdlan | AZCL-pachyman |
| Bacillus species B | 0.059^{*1} | 0.7 | 0.629^{*1} | 0.831 |
| Paenibacillus sp | 0.153 | 0.7 | 0.201 | 0.693 |
| Thermobacillus sp | 0.153 | 0.7 | 0.579 | 0.815 |
| Cohnella sp | 0.153 | 0.7 | 0.216 | 0.707 |
| Paenibacillus elgii | 0.059^{*1} | 0.7 | 0.386^{*1} | 1.047 |
| Bacillus species A | 0.059^{*1} | 0.7 | 1.394^{*1} | 0.880 |

^{*1}diluted 5 times

### Example 5: Activity Measurement of Beta-Glucanases (Laminarinases) Using a Reducing End Assay

### I. Reducing End Method Description

Laminarin (from Sigma-Aldrich) or Yeast barley beta-glucan from Megazyme (100 mg) was suspended in 100 mL detergent (Model detergents A, X or ADW Model A), heated to 70° C. and stirred overnight. To 1 mL of this solution in Eppendorf tubes was added 25 μL enzyme (0.5 mg enzyme protein per milliliter), incubated for 60 min at 37° C. while shaking at 1250 rpm in a pre-heated thermos-mixer and stored on ice. 50 μL of the incubated sample was transferred to Eppendorf tubes and 250 μL PAHBAH-reagent mixture (Reagent A:100 μL+Reagent B: 900 μL—see descriptions below) was added and incubated for 6 min at 60° C. in thermo-mixer. Then placed on ice for 3 min, followed by 3 min at room temperature. Then 150 μL of the solution was transferred to a micro-titer plate and the sample absorbance was measured at 410 nm, either measured directly or diluted 2 or 3 times with a 5% Triton-X-100 including 10 μM CaCl₂. Reagent A: To MilliQ water (6 mL) was added p-hydroxy benzoic acid hydrate (PAHBAH, 1 g), then HCl-solution (3 mL, 4 M) and then MilliQ water was added up to 20 mL.

Reagent B: To MilliQ water (50 mL) was added trisodium citrate (2.49 g), CaCl₂, 2H₂O (0.268 g), sodium hydroxide (0.268 g) and then MilliQ water was added up to 200 mL.

### II. Results

In this example enzymatic activity were measured on laminarin and yeast beta-glucan substrate in detergents thus modeling various laundry and automatic dish wash and hand wash conditions. Measurements of enzymatic activity were carried out as in the reducing end method, above.

TABLE 5
Reducing end assay in Model Detergent A
Model detergent A

| | Absorbance at 410 nm | | Absorbance at 410 nm | |
|---|---|---|---|---|
| | (Blank) | | (Laminarinase) | |
| | | Yeast | | Yeast |
| Enzyme | Laminarin^{*1} | beta-glucan | Laminarin^{*1} | beta-glucan |
| Bacillus species B | 0.404 | 0.309 | 1.087 | 0.274 |
| Paenibacillus sp | 0.404 | 0.309 | 1.157 | 2.296 |
| Thermobacillus sp | 0.404 | 0.309 | 1.135 | 0.508 |
| Cohnella sp | 0.404 | 0.309 | 1.184 | 2.056 |
| Paenibacillus elgii | 0.404 | 0.309 | 1.138 | 2.112 |
| Bacillus species A | 0.404 | 0.309 | 1.110 | 0.977 |

^{*1}diluted 3 times.

TABLE 6
Reducing end assay in Model Detergent X
Model detergent X

| | Absorbance at 410 nm | | Absorbance at 410 nm | |
|---|---|---|---|---|
| | (Blank) | | (Laminarinase) | |
| | | Yeast | | Yeast |
| Enzyme | Laminarin | beta-glucan | Laminarin | beta-glucan |
| Bacillus species B | 0.411^{*1} | 0.323 | 1.285^{*1} | 0.260 |
| Paenibacillus sp | 1.133 | 0.323 | 1.617 | 1.288 |
| Thermobacillus sp | 1.133 | 0.323 | 1.462 | 0.25 |
| Cohnella sp | 1.133 | 0.323 | 0.975 | 0.307 |
| Paenibacillus elgii | 1.133 | 0.323 | 1.778 | 1.459 |
| Bacillus species A | 0.411^{*1} | 0.323 | 1.287^{*1} | 0.292 |

^{*1}diluted 3 times

TABLE 7
Reducing end assay in ADW Model Detergent A
ADW Model detergent A

| | Absorbance at 410 nm | | Absorbance at 410 nm | |
|---|---|---|---|---|
| | (Blank) | | (Laminarinase) | |
| | | Yeast | | Yeast |
| Enzyme | Laminarin | beta-glucan | Laminarin | beta-glucan |
| Bacillus species B | 0.559 | −0.065 | 1.528 | 0.181 |
| Paenibacillus sp | 0.559 | 0.62 | −0.005 | 0.866 |
| Thermobacillus sp | 0.559 | −0.075 | 0.260 | 0.171 |
| Cohnella sp | 0.559 | −0.056 | −0.069 | 0.19 |
| Paenibacillus elgii | 0.559 | 0.457 | 0.187 | 0.703 |
| Bacillus species A | 0.559 | 0.015 | 1.611 | 0.261 |

### Example 6: Beta-Glucanase Substrate Specificity

The substrate specificities of beta-glucanases were further tested using various assays (AZCL-curdlan, AZCL-pachyman (azurine dye covalently cross-linked beta-glucan from Megazyme), laminarin (from Sigma-Aldrich) and yeast beta-glucan (from Megazyme), generally following the protocol as described in Example 1 and Example 5, above.

In this example substrate specificity of the beta-glucanases of the invention were tested on various substrates. Based on this substrate specificity, it is concluded that the beta-glucanases of the present invention have laminarinase enzymatic activity.

TABLE 8
Substrate specificity of laminarinases
Substrate specificity of laminarinases

| | | Substrate for | |
|---|---|---|---|
| Substrate | Reaction | the assay of: | Polymer description |
| AZCL-curdlan | Yes | Endo-1,3-beta- | β-1,3 linkages between |
| | | D-glucanase | D-glucose |
| AZCL-pachyman | Yes | Endo-1,3-beta- | β-1,3 linkages between |
| | | D-glucanase | D-glucose units (branched |
| | | | with very few β-1,6 |
| | | | glucose units) |
| Laminarin | Yes | Endo-1,3-beta- | β-1,3 linkages between D- |
| | | D-glucanase | glucose units (branched |
| | | | with β-1,6 glucose units |
| | | | average on approximate |
| | | | every 7) |
| Yeast beta-glucan | Yes^{*1} | Endo-1,3-beta- | β-1,3 linkages between D- |
| | | D-glucanase | glucose units (branched |
| | | | with β-1,6 glucose units |
| | | | average on approximate |
| | | | every 4) |

^{*1}No/very little activity of Bacillus species B was shown on this substrate under the tested conditions.

In addition, NMR analyses (data not shown) were performed of the Bacillus species B, Paenibacillus sp, Thermobacillus sp, Cohnella sp, Paenibacillus elgii, and Bacillus species A beta-glucanases according to the invention and their degradation of mixed linkage (1,3;1,4) beta-glucan. All except for Bacillus species B act on the substrates, whereas Bacillus species B only reacts a little. Based on this, we conclude that Bacillus species B can be classified as EC 3.2.1.39, whereas the other five enzymes can be classified as EC 3.2.1.6.

### Example 7: Synergistic Effect of Beta-Glucanases (Laminarinases) of the Invention when Combined with an Alpha-Amylase

### I. Wascator Bottle Wash Method Description

A Wascator bottle wash method was used to detect the performance of the enzymes. In a Wascator washing machine (FOM 71 Lab) bottles (60 mL, DSE PP 70X35 Aseptisk, material No.: 216-2620, from VWR) with 25 mL detergent solution including enzyme(s) and four stains (035KC Chocolate porridge oat from Equest, 2 cm in diameter) were added. Two kg ballast (tea towels, cotton) was included in the washing machine. Washed in 25 L water for 20 min at 29° C. in liquid model detergent for laundry (model detergent A). After wash the stains were rinsed with tap water twice (3 L) and dried overnight at rt (room temperature) in drying cabinet (Electrolux, Intuition, EDD2400). The remission was measured on a spectrophotometer (Macbeth Color-Eye 7000 Remissions) at 460 nm.

### II. Results

In this example the results of combining the individual laminarinases with an alpha-amylase (SEQ ID NO: 45) were studied in order to investigate a potential synergistic effect between the two enzymes using the Wascator bottle wash method, in the presence of 0.1 mg enzyme protein per liter of laminarinase and 0.1 mg enzyme protein per liter of alpha-amylase (SEQ ID NO: 45) at 29° C. The detailed conditions used in this example are described in Table 9 below and the results are shown in Table 10 below.

TABLE 9
Experimental conditions
Experimental conditions

| Detergent | Model detergent A (as above) |
|---|---|
| Detergent dosage | 3.33 g/L |
| Test solution volume | 25 mL |
| pH | As is |
| Wash time | 20 minutes |
| Temperature | 29° C. |
| Water hardness | 15° dH |
| Alpha-amylase | 0 or 0.1 mg/L |
| concentration in test | |
| Beta-glucanase | 0 or 0.1 mg/L |
| (laminarinase) | |
| concentration in test | |
| Test material | O35 KC Chocolate porridge oats |

### Abbreviations as Used Herein

REM=Measured value
ΔREM=REM—Blank
REM combined=Measured value
ΔREM combined=REM combined—Blank
ΔREM theoretic=ΔREM (Amylase)+ΔREM (Laminarinase)
REM Synergistic effect=ΔREM combined—ΔREM theoretic

TABLE 10
Wascator bottle wash in Model A at 29° C., 20 min (pH 7.7)

| | | | Laminarinase in combination | | | |
|---|---|---|---|---|---|---|
| | | | with alpha-amylase | | | |
| | | | | | | REM |
| | Enzymes | | REM | ΔREM | ΔREM | Syner- |
| | solo | | com- | com- | theo- | gistic |
| | REM | ΔREM | bined | bined | retic | effect |
| Bacillus species B | 53.8 | −0.9 | 66.7 | 12.0 | 8.8 | 3.2 |
| Cohnella species | 53.8 | −1.2 | 72.0 | 17.3 | 8.5 | 8.8 |
| Paenibacillus elgii | 54.0 | −0.7 | 72.3 | 17.6 | 9.0 | 8.6 |
| Paenibacillus | 55.4 | 0.7 | 74.5 | 19.8 | 10.4 | 9.4 |
| species | | | | | | |
| Thermobacillus | 54.5 | −0.2 | 66.3 | 11.6 | 9.5 | 2.1 |
| species | | | | | | |
| Bacillus species A | 55.3 | 0.6 | 70.4 | 15.7 | 10.3 | 5.4 |
| Alpha-amylase | 64.5 | 9.7 | — | — | — | — |
| Blank | 54.7 | 0.0 | — | — | — | — |

### Example 8: Full Scale Wash

This test method evaluates wash performance in full scale wash under EU conditions (washing in a front loader washing machine).

The real items (T-shirts) and ballast are added to each wash together with detergent and enzyme. After wash, the real items (T-shirts) are dried. After drying, color difference of the round swatches round is measured on a MacBeth Color Eye 7000 Remissions spectrophotometer at 460 nm.

The enzymes are added on basis of weight percent of the detergent dosage in each wash,

### Equipment Used:

Washing machine: Miele Laundry Washing Machines W1935
Water meters and automatically data collection system
MacBeth Color Eye 7000 Remissions spectrophotometer

### For the Preparation and Adjustment of Water Hardness the Following Ingredients are Needed:

Calcium chloride (CaCl₂·2H₂O)
Magnesium chloride (MgCL₂·6H₂O)
Sodium Hydrogen Carbonate (NaHCO₃)

### Ballast

The ballast consists of clean white cloth without optical whitener made of cotton, polyester or cotton/polyester. The composition of the ballast is a mix of different items at a cotton/polyester ratio of 65/35 based on weight. The ballast weight, dryness and item composition must be the same in each wash.

After each wash the ballast is inactivated in an industrial washer at 85° C./15 min or in a 95° C. wash (EU machine) without detergent

Ballast and tea towel with stains (Composition, total 4 kg)
3 T-shirts (100% cotton)
12 shirts, short sleeves (55% cotton 45% polyester)
7 pillow cases (35% cotton, 65% polyester), 110×75 cm
3 small bed sheets, size 100×75 cm (100% cotton)
3 Tea towels (100% cotton)

### Wash Conditions

Temperature: 20° C. & 40° C.
Washing programme: Cottons, activate “short” and “water plus”, spin speed 1600
Water level 15.6 L with “water plus”
Water hardness: Standard EU conditions: 15° dH, Ca2+:Mg2+:HCO₃=4:1:7.5
Amylase (SEQ ID NO: 45) dosage: 0.1 mg/L
Laminarinase (Paenibacillus elgii): 0.1 mg/L
Detergent: Model Detergent A (as above)

### Detailed Steps to Carry Out Full Scale Wash Trial

1. Select wash program as in study plan.
2. The detergent and enzyme(s) are placed in the wash drum in a “washing ball” (both liquid and powder detergents). Place it at the bottom.
3. Place the real items (T-shirts) and ballast in the wash drum.
4. Start digital water meter
5. Start the washer by pressing the knob START
6. After wash, take out real items (T-shirts) and ballast, put real items into drying room.

### Drying Procedure

Put stains on tray or hang in line and dry at room temperature. The room has a dehumidifier working for 24 hours per day to keep the room dry.

### Measurement

After wash the stains are removed from tea towels and dried overnight at rt (room temperature) in drying cabinet (Electrolux, Intuition, EDD2400). The remission was measured on a spectrophotometer (Macbeth Color-Eye 7000 Remissions) at 460 nm.

### Swatches

The swatches include a combination of food and technical stains.

| Material | Source |
|---|---|
| 035KC Chocolate Porridge Oats | Warwick Equest |
| Cosmetic beta-glucan C | Warwick Equest |
| Liken Deodorant HY | Warwick Equest |

The above commercial test materials are purchased from Warwick Equest Ltd, Unit 55, Consett Business Park, Consett, County Durham, DH8 6BN, United Kingdom, 5 cm in diameter.

### Results

| | REM | | REM (Amylase + | |
|---|---|---|---|---|
| | (Amylase only) | | Laminarinase) | |
| Stain | 20° C. | 40° C. | 20° C. | 40° C. |
| 035KC Chocolate Porridge Oats | 65.4 | 69.8 | 83.3 | 81.0 |
| Cosmetic beta-glucan C | 49.9 | 51.5 | 60.1 | 59.0 |
| Liken Deodorant HY | 71.8 | 86.9 | 76.0 | 85.5 |

### Example 9: Full Scale Wash and Red Clay Treatment

This test method evaluates wash performance in full scale wash under EU conditions (washing in a front loader washing machine).

The real items (T-shirts) and ballast are added to each wash together with detergent and enzyme. After wash, the real items (T-shirts) are dried. After drying, color difference of the round swatches round is measured on a MacBeth Color Eye 7000 Remissions spectrophotometer at 460 nm.

The enzymes are added on basis of weight percent of the detergent dosage in each wash.

### Equipment Used:

Washing machine: Miele Laundry Washing Machines W1935
Water meters and automatically data collection system
MacBeth Color Eye 7000 Remissions spectrophotometer

### For the Preparation and Adjustment of Water Hardness the Following Ingredients are Needed:

Calcium chloride (CaCl₂·2H₂O)
Magnesium chloride (MgCL₂·6H₂O)
Sodium Hydrogen Carbonate (NaHCO₃)

### Ballast

The ballast consists of clean white cloth without optical whitener made of cotton, polyester or cotton/polyester. The composition of the ballast is a mix of different items at a cotton/polyester ratio of 65/35 based on weight. The ballast weight, dryness and item composition must be the same in each wash.

After each wash the ballast is inactivated in an industrial washer at 85° C./15 min or in a 95° C. wash (EU machine) without detergent

Ballast and Tea Towel with Stains (Composition, Total 4 kg)
3 T-shirts (100% cotton)
12 shirts, short sleeves (55% cotton 45% polyester)
7 pillow cases (35% cotton, 65% polyester), 110×75 cm
3 small bed sheets, size 100×75 cm (100% cotton)
3 Tea towels (100% cotton)

### Wash Conditions

Temperature: 30° C.
Washing programme: Cottons, activate “short” and “water plus”, spin speed 1600
Water level 15.6 L with “water plus”
Water hardness: Standard EU conditions: 15° dH, Ca2+:Mg2+:HCO3=4:1:7.5
Amylase (SEQ ID NO: 45) dosage: 0.2 mg/L
Laminarinase (Paenibacillus elgii): 0.1 mg/L
Detergent: Model detergent A

### Detailed Steps to Carry Out Full Scale Wash Trial

1. Select wash program as in study plan.
2. The detergent and enzyme(s) are placed in the wash drum in a “washing ball” (both liquid and powder detergents). Place it at the bottom.
3. Place the real items (T-shirts) and ballast in the wash drum.
4. Start digital water meter
5. Start the washer by pressing the knob START
6. After wash, take out real items (T-shirts) and ballast, put real items into drying room.

### Drying Procedure

Put stains on tray or hang in line and dry at room temperature. The room has a dehumidifier working for 24 hours per day to keep the room dry.

After FSW, red clay treatment was conducted on the real items to visualize differences more.

### Red Clay Procedure in Terg-O-Tometer (TOM) Wash Assay

The Tergo-To-Meter (TOM) is a medium scale model wash system that can be applied to test 16 different wash conditions simultaneously. A TOM is basically a large temperature controlled water bath with up to 16 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments, such as AMSA and mini-wash, and the more time consuming full scale experiments in top loader washing machines.
1. 500 mL tap water and 1 Red Clay and Ferric Oxide (RC) stain. Stir in TOM 5 min (23° C., 120 rpm). Remove RC stain
2. Add the wanted stains to beaker
3. Stir 5 min (23° C., 120 rpm)
4. Remove stains and rinse in tap water
5. Dry at room temperature
6. The remission was measured on a spectrophotometer (Macbeth Color-Eye 7000 Remissions) at 460 nm.

### Swatches

The swatches include a combination of food and technical stains.

| Material | Source |
|---|---|
| 035KC Chocolate Porridge Oats | Warwick Equest |
| 081KC Chocolate milkshake (Frijj) | Warwick Equest |
| 113KC Spaghetti Sauce | Warwick Equest |
| 138kC Campbells Tomato Soup | Warwick Equest |
| KBBQ (Kraft BBQ sauce) | Warwick Equest |
| NZ100KC Red Clay and Ferric Oxide | Warwick Equest |

The above commercial test materials are purchased from Warwick Equest Ltd, Unit 55, Consett Business Park, Consett, County Durham, DH8 6BN, United Kingdom, 5 cm in diameter.

### Results

| | | REM after Red |
|---|---|---|
| | REM after Red | Clay treatment |
| | Clay treatment | (Amylase + |
| Stain | (Amylase only) | Laminarinase) |
| 035KC Chocolate | 46.4 | 63.0 |
| Porridge Oats | | |
| 081KC Chocolate | 46.7 | 49.3 |
| milkshake (Frijj) | | |
| 113KC Spaghetti Sauce | 54.8 | 59.0 |
| 138kC Campbells | 54.0 | 57.7 |
| Tomato Soup | | |
| KBBQ (Kraft BBQ sauce) | 56.5 | 58.8 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A cleaning composition comprising a polypeptide having beta-glucanase activity, wherein the polypeptide is a gram-positive bacteria of order Bacillales and comprises a motif selected from the group consisting of NXAXGG (SEQ ID NO: 30), GEXDXME (SEQ ID NO: 28), GXGNXEXXXY (SEQ ID NO: 29), YTS[G/A][K/R](SEQ ID NO: 31) and combinations thereof, and at least one cleaning component.

2. The composition of claim 1, wherein said beta-glucanase activity is laminarinase activity EC 3.2.1.6, EC 3.2.1.39, or EC 3.2.1.58.

3. The composition of claim 1, wherein the polypeptide has endo-1,3-beta-glucanase activity, e.g., EC 3.2.1.6 or EC 3.2.1.39.

4. (canceled)

5. The composition of claim 1, wherein the polypeptide comprises a motif selected from the group consisting of [L/M]NXAXGG, LNXAXGG (SEQ ID NO: 43), GEIDIME (SEQ ID NO: 32), G[F/W]GNXEX[Q/E]XY (SEQ ID NO: 33), and combinations thereof.

6. The composition of claim 1, wherein the polypeptide comprises each of the motifs LNXAXGG (SEQ ID NO: 43), GXGNXEXXXY (SEQ ID NO: 29), and GEXDXME (SEQ ID NO: 28).

7. The composition of claim 1, wherein the polypeptide comprises, consists, or consists essentially of a polypeptide having an amino acid sequence selected from the group consisting of SEQ ID NO: 12, SEQ ID NO: 3, SEQ ID NO: 6, SEQ ID NO: 9, SEQ ID NO: 15, and SEQ ID NO: 18, or a polypeptide having at least 60% identity thereto.

8-17. (canceled)

18. The composition of claim 1, wherein said composition has pH of 7.5 or above and optionally comprises a bleaching agent.

19-35. (canceled)

36. A process of degrading a beta-glucan comprising applying the cleaning or detergent composition or polypeptide of claim 1 to said beta-glucan; optionally, said process is carried out under alkaline conditions having pH 7.5 or above.

37. (canceled)


SEQUENCE LISTING (94 sequences)

SEQ ID NO: 1 - DNA, 1551 bp, Thermobacillus sp.
  sig_peptide 1..108
  mat_peptide 109..1548
  source 1..1551: mol_type = genomic DNA
  CDS 1..1548
atgttcggacgcaatcgggtcaaggtgtgtttcttgatggcgcttggtctcgttttgacgatggggatggtgcctttgccctttgtcccttccgcgaaatcccacgccgccgacttaagccagtggagattggtgtggagtgacgagtttgacggccccaacgggtccgcgcccgatccgaataagtggaatcttgttcatgcgggcggcggtttcggcaacaatgaacttcagtactacacgaatcgccgggacaattcttacctggaaaacggctctttggtcatcaaggcccagaaagaaacgtacaacggacacgcttatacctcggcaaagctgacgtcgcaaaacaagggcgattggaaatacgggagattcgaaattcgcgccaaattgccctacgggagaagcgtatggcccgcgatctggatgatgccgacggattcggtttacggcggctggccgaagagcggcgaaatcgatatcatggaaaaccgcggcgatcaaatgaacaaaatttcgggcaccatccattacggcaatgattggccgaacaacacgtggtccggcgcaagctataatctgccgggcggtcaatcctttgccgatgattttcacaccttcgcgatcgaatgggaggaagggatcatccgttggtacgtggatgacattctctactcgacgaaaacggattggttcacgccttccgcaccttatccggcccccttcgaccaaaggttttacatgcagctcaatgtggcgatcggcggccccaacacgccgttcaccggttttcaaccccccgatgactcggtgcttccgcaaaaaatgtatgtcgactatgttcgcgtatatgaacggatcggctcgtcgtcgtcgacaccgattcccgggaaaatcgaagcggaaaactacagcgccatgaacgggattcaaacggagcaaacgaccgacaccggcggcggtctcaatgtcggatgggtggacgccggggattggctggattattcggtcaacgtccaaacttccggcacctacaaggttcaattgcgcgtggcgaacgcgctcagcaccggccagcttcagctgcgatccggcggcacaacgctggcaacggtgaatgtgcccaatacgggcggatggcaaagctggcaaactatcgaaacgaccgtcaacttgacggcagggcaacaaaccctcagagtctacgcgacccaaaggggatttaacctgaactggctgaactttatccaggacggcggttcgggaggcggcggcgaacacgtaacggcggattataccgccggagtgtctcgcgtaagcgccagccaggcgaagatatactttacacctgtgacgccggcaagatatgtggacgtccattatacggtcaacagcggcggtcagctgaatgtcagaatgacgaataacaacggaacgtgggaaacggcggtaaacaatttgaagtcgggagatgtcatccgttattggtttacctacgagaaaaacggcccgcaatacgaatctcccgaatacacttacactcattaa

SEQ ID NO: 2 - protein, 516 aa, Thermobacillus sp.
MFGRNRVKVCFLMALGLVLTMGMVPLPFVPSAKSHAADLSQWRLVWSDEFDGPNGSAPDPNKWNLVHAGGGFGNNELQYYTNRRDNSYLENGSLVIKAQKETYNGHAYTSAKLTSQNKGDWKYGRFEIRAKLPYGRSVWPAIWMMPTDSVYGGWPKSGEIDIMENRGDQMNKISGTIHYGNDWPNNTWSGASYNLPGGQSFADDFHTFAIEWEEGIIRWYVDDILYSTKTDWFTPSAPYPAPFDQRFYMQLNVAIGGPNTPFTGFQPPDDSVLPQKMYVDYVRVYERIGSSSSTPIPGKIEAENYSAMNGIQTEQTTDTGGGLNVGWVDAGDWLDYSVNVQTSGTYKVQLRVANALSTGQLQLRSGGTTLATVNVPNTGGWQSWQTIETTVNLTAGQQTLRVYATQRGFNLNWLNFIQDGGSGGGGEHVTADYTAGVSRVSASQAKIYFTPVTPARYVDVHYTVNSGGQLNVRMTNNNGTWETAVNNLKSGDVIRYWFTYEKNGPQYESPEYTYTH

SEQ ID NO: 3 - protein, 480 aa, Thermobacillus sp.
ADLSQWRLVWSDEFDGPNGSAPDPNKWNLVHAGGGFGNNELQYYTNRRDNSYLENGSLVIKAQKETYNGHAYTSAKLTSQNKGDWKYGRFEIRAKLPYGRSVWPAIWMMPTDSVYGGWPKSGEIDIMENRGDQMNKISGTIHYGNDWPNNTWSGASYNLPGGQSFADDFHTFAIEWEEGIIRWYVDDILYSTKTDWFTPSAPYPAPFDQRFYMQLNVAIGGPNTPFTGFQPPDDSVLPQKMYVDYVRVYERIGSSSSTPIPGKIEAENYSAMNGIQTEQTTDTGGGLNVGWVDAGDWLDYSVNVQTSGTYKVQLRVANALSTGQLQLRSGGTTLATVNVPNTGGWQSWQTIETTVNLTAGQQTLRVYATQRGFNLNWLNFIQDGGSGGGGEHVTADYTAGVSRVSASQAKIYFTPVTPARYVDVHYTVNSGGQLNVRMTNNNGTWETAVNNLKSGDVIRYWFTYEKNGPQYESPEYTYTH

SEQ ID NO: 4 - DNA, 873 bp, Paenibacillus sp.
  sig_peptide 1..84
  mat_peptide 85..870
  source 1..873: mol_type = genomic DNA
  CDS 1..870
gtgaagagacttttcaagatttcgagtatttttctagcaattatcttatttataatggggtgcagcatggaaaataaaaaagaagctccgcagcagaaaaatgggtggaagctcgtatggaacgatgagtttgatggcaaagaaatcgatcggaccaaatggaagcatgtaaccggaggatcaggcttcgggaataatgaagatcagttttatacagaggatgcggccaactcttatattgaagatggcaagctagtcatcaaagcgttgaagcaggagcatgggggcaagccgtatacgtccgcgaaattaattacggaaggatatgcggactggacatacggccgattcgaattccgggctaagatgccgcttggcaaaggaatgtggcctgccatatggatgatgccgacggacatggagaaatacggcggctggccaagcagcggcgagattgacattatggagtatcttggacacgagcctgagcaggtgcatggtactttgcatatgggcaaccctcacatttatcgaggcgggaaggtaagtctggaagatggaatgttcgcagaagcgtttcatgactttgctctggaatggacgccaagcgggatgaagtggtacgtcgacgataaggagttttatcagacgacgacatggtttacgcgcaaggatgaggctgcggataatgagccattccctgcacctttcgatcgtgcgtttttcttgcagctgaatttggccgttggcggcaattggccgggttatccagatgagacgacggttttcccgcaaacattcgagctggagtacgttcgggtgtatcagccggcagacggaaattatgagacggcaaacgatacagcgaaatag

SEQ ID NO: 5 - protein, 290 aa, Paenibacillus sp.
VKRLFKISSIFLAIILFIMGCSMENKKEAPQQKNGWKLVWNDEFDGKEIDRTKWKHVTGGSGFGNNEDQFYTEDAANSYIEDGKLVIKALKQEHGGKPYTSAKLITEGYADWTYGRFEFRAKMPLGKGMWPAIWMMPTDMEKYGGWPSSGEIDIMEYLGHEPEQVHGTLHMGNPHIYRGGKVSLEDGMFAEAFHDFALEWTPSGMKWYVDDKEFYQTTTWFTRKDEAADNEPFPAPFDRAFFLQLNLAVGGNWPGYPDETTVFPQTFELEYVRVYQPADGNYETANDTAK

SEQ ID NO: 6 - protein, 262 aa, Paenibacillus sp.
APQQKNGWKLVWNDEFDGKEIDRTKWKHVTGGSGFGNNEDQFYTEDAANSYIEDGKLVIKALKQEHGGKPYTSAKLITEGYADWTYGRFEFRAKMPLGKGMWPAIWMMPTDMEKYGGWPSSGEIDIMEYLGHEPEQVHGTLHMGNPHIYRGGKVSLEDGMFAEAFHDFALEWTPSGMKWYVDDKEFYQTTTWFTRKDEAADNEPFPAPFDRAFFLQLNLAVGGNWPGYPDETTVFPQTFELEYVRVYQPADGNYETANDTAK

SEQ ID NO: 7 - DNA, 1251 bp, Cohnella sp.
  sig_peptide 1..93
  mat_peptide 94..1248
  source 1..1251: mol_type = genomic DNA
  CDS 1..1248
atgttgcacgtgttcaagaagaggaaggcgtttctgctggtgctggtgttcgctctcgcggccgcgctgctgccgatctcgagcgcgaacgcggcgtacaacctggtctggagcgatgagttcaacggaacgtccatcgattcgaacaactggtcgttcgaggtcggaacgggcagcggaggctggggcaacaacgaactcgaatactatacaagccggtcggagaacgcccggatcgagaacggcaacctggtcatcgaagcgcggaaggaatcctatggcgggatgaattacacctcggcccgcttgaagacgcagggcaagaagagcttccaatacggccggatcgaggcccgcatcaagatgccgaatggccaggggctgtggccggctttctggacgcttggttcggacatcggcacggtcggctggccgaagagcggcgagatcgacatcatggagcatgtgaacaacgacaataataccaacgggtacattcattgggacgccaacggacaagccgactacggcgggcccagcgggtacgtggacgtaacccagtaccatgtgtattcgatcgagtggacgccgagcgcgatcaagtggttcatcgacggcacccagttctgggaagccaacatcgcgaacaacatcaactcgacggaggagttccacaagccgcattttattctgctgaacatggccgtcggcggcaactggccgggcgctcccaatgccggcacggcgttcccggcgaagatgtacgtcgattacgtgcgggtctatcaagacaacggaacgccgcagccttcgaacgggatcgtgtcgggcggcacgtacaagctgatcaacgtgaacagcggcaaagcgctggacgttcagagcgccggaacgacgccgggaaccaatgtccagatctggacggacaacggaacgggcgctcagaagtggacgatctatcagaacgcggacggctcgtataagctcgtgaacgtcaatagcgcgctagctttggacgtggcctcgtccggcaccgcggacgggacgaacgtgcaggcttggacggataacgggacgggcgcgcagaagtggaacatcatcgcgaacggggacggctcctacaagctgatcaacacgaacagcggcaaggcgttggacgtctccggatcgggcacggcggacgggacgaacgtccagatctggaacgacaacggcaccggcgcgcaaaagtggaatttgattaagctgtaa

SEQ ID NO: 8 - protein, 416 aa, Cohnella sp.
MLHVFKKRKAFLLVLVFALAAALLPISSANAAYNLVWSDEFNGTSIDSNNWSFEVGTGSGGWGNNELEYYTSRSENARIENGNLVIEARKESYGGMNYTSARLKTQGKKSFQYGRIEARIKMPNGQGLWPAFWTLGSDIGTVGWPKSGEIDIMEHVNNDNNTNGYIHWDANGQADYGGPSGYVDVTQYHVYSIEWTPSAIKWFIDGTQFWEANIANNINSTEEFHKPHFILLNMAVGGNWPGAPNAGTAFPAKMYVDYVRVYQDNGTPQPSNGIVSGGTYKLINVNSGKALDVQSAGTTPGTNVQIWTDNGTGAQKWTIYQNADGSYKLVNVNSALALDVASSGTADGTNVQAWTDNGTGAQKWNIIANGDGSYKLINTNSGKALDVSGSGTADGTNVQIWNDNGTGAQKWNLIKL

SEQ ID NO: 9 - protein, 385 aa, Cohnella sp.
AYNLVWSDEFNGTSIDSNNWSFEVGTGSGGWGNNELEYYTSRSENARIENGNLVIEARKESYGGMNYTSARLKTQGKKSFQYGRIEARIKMPNGQGLWPAFWTLGSDIGTVGWPKSGEIDIMEHVNNDNNTNGYIHWDANGQADYGGPSGYVDVTQYHVYSIEWTPSAIKWFIDGTQFWEANIANNINSTEEFHKPHFILLNMAVGGNWPGAPNAGTAFPAKMYVDYVRVYQDNGTPQPSNGIVSGGTYKLINVNSGKALDVQSAGTTPGTNVQIWTDNGTGAQKWTIYQNADGSYKLVNVNSALALDVASSGTADGTNVQAWTDNGTGAQKWNIIANGDGSYKLINTNSGKALDVSGSGTADGTNVQIWNDNGTGAQKWNLIKL

SEQ ID NO: 10 - DNA, 1233 bp, Paenibacillus elgii
  sig_peptide 1..84
  mat_peptide 85..1230
  source 1..1233: mol_type = genomic DNA
  CDS 1..1230
atgttgaaaagaggaaagctgctaagtttttttctgctgtttactctcctggtcgccctggtgcccgtcggcggtgcgaatgcggccccgaactggaatctggtctggagcgacgaatttaacggcaatgctttaaattccgccaactggtctgcggaaatcggcacgggccagaacgggtggggaaacaatgagctgcagtattatacgaaccggcccgaaaatgtgcgggttgcggacgggaatctcgtcattacggcgcgcaaggagtcttatggcggcatgaattacacctctgcgcggatcaaaacccaaaatttgaaaagctttacttacgggaaaatcgaagcgcgtatcaagctgccctccggtcaagggctgtggccggcattctggatgctgggatcgaacatcaatgcggtgggctggcctgggtgcggcgagatcgacattatggaacgggtcaacaacaacccgcatgtcaacggcaccgtacactgggatgcgggaggacacgccgattacgggcgggtttccggcaatctcgatttttcccagtatcacgtgtacagcatcgagtgggactccaaatatatccgatggtttgtggacggacaggagtacaacgctttttatattgaaaatggcaccgggaacacggaggagtttcaacggccgttcttcctcctgctgaatcttgccgtgggcggcaattggcccggcagccccgatccttccacaccgttcccggcgcaaatgctggtggattacgtgcgcgtgtaccaagcttcgccgggtattgtcaacggaggcatttatacgatagcctccaaggccagcggcaaggtgatggatgtcgtggacgtgtcgacgcagagcggggccaaaatacagcaatggaccaactacgtcgccaataaccagaagttcaaggtggagagcgccggggacggctactacaagctcacggccgttcacagcggaaaagtgctggatgtgcccaactcgtcgacttccagcggcgtgcagctgcagcaatgggacgataacggcagcgctgcgcaaaggtggagtatcgtcgatgcgggcggcggctattataagattgtttcaaaaacgaacgggctcgtcgtggatgtatccggttcctcgaccgccgacggcgcgacggtccagcagtggagcgacaatggaacggatgcgcaaaagtggtcgtttgtcaaagtcaactga

SEQ ID NO: 11 - protein, 410 aa, Paenibacillus elgii
MLKRGKLLSFFLLFTLLVALVPVGGANAAPNWNLVWSDEFNGNALNSANWSAEIGTGQNGWGNNELQYYTNRPENVRVADGNLVITARKESYGGMNYTSARIKTQNLKSFTYGKIEARIKLPSGQGLWPAFWMLGSNINAVGWPGCGEIDIMERVNNNPHVNGTVHWDAGGHADYGRVSGNLDFSQYHVYSIEWDSKYIRWFVDGQEYNAFYIENGTGNTEEFQRPFFLLLNLAVGGNWPGSPDPSTPFPAQMLVDYVRVYQASPGIVNGGIYTIASKASGKVMDVVDVSTQSGAKIQQWTNYVANNQKFKVESAGDGYYKLTAVHSGKVLDVPNSSTSSGVQLQQWDDNGSAAQRWSIVDAGGGYYKIVSKTNGLVVDVSGSSTADGATVQQWSDNGTDAQKWSFVKVN

SEQ ID NO: 12 - protein, 382 aa, Paenibacillus elgii
APNWNLVWSDEFNGNALNSANWSAEIGTGQNGWGNNELQYYTNRPENVRVADGNLVITARKESYGGMNYTSARIKTQNLKSFTYGKIEARIKLPSGQGLWPAFWMLGSNINAVGWPGCGEIDIMERVNNNPHVNGTVHWDAGGHADYGRVSGNLDFSQYHVYSIEWDSKYIRWFVDGQEYNAFYIENGTGNTEEFQRPFFLLLNLAVGGNWPGSPDPSTPFPAQMLVDYVRVYQASPGIVNGGIYTIASKASGKVMDVVDVSTQSGAKIQQWTNYVANNQKFKVESAGDGYYKLTAVHSGKVLDVPNSSTSSGVQLQQWDDNGSAAQRWSIVDAGGGYYKIVSKTNGLVVDVSGSSTADGATVQQWSDNGTDAQKWSFVKVN

SEQ ID NO: 13 - DNA, 855 bp, Bacillus sp.
  sig_peptide 1..72
  mat_peptide 73..852
  source 1..855: mol_type = genomic DNA
  CDS 1..852
atgaagaaaatgcttggcacattatcaatcacagcatgtgcaacatttattgcaacctcttatgttggcgcaaccgaaacagaagaaggcatttccatagaacaagaaggatggaacctcgtttggaatgatgaatttgatggagattccttagaccaatcaaagtggcgctatgatattgggaacggacaacccaatttaccaggttggggcaatgaggaattacaatattacaatgatgatccaaaaaatgttcgcgtggaaaacggtgagctaattattgaagcacatcaagaaccgatttccgatgaattcggtagctacgagtatacgtctggcaaagtgttaacagaaggactgtttagtcaaacttacgggcgatttgaggcacggatgcgtttaccagcgggacaagggttttggccagctttttggatgatgccagaaaacgatcagtatggtggttgggctgcttcaggggaaattgatattatggaaaacgcgggaggaacaccttacaaagtcggaggagcgatccactatggcgggccttggcctgagaatcaattccaagctggcgactatttctttcctgatggcaccgatgcaaccggttatcacgaatacgcggttgaatgggaaccaggcgaaattcgttggtacgttgatgggaacttgtatcaaacaattaacgactggtattcaacaggtggcgcgtaccctgccccattcgatcaagatttccacttaatccttaaccttgctgttggcggctggtacggcgggaatccagatggttcaacgccattcccatcgacaatggctgttgattacgttcgagtgtacgaaagataa

SEQ ID NO: 14 - protein, 284 aa, Bacillus sp.
MKKMLGTLSITACATFIATSYVGATETEEGISIEQEGWNLVWNDEFDGDSLDQSKWRYDIGNGQPNLPGWGNEELQYYNDDPKNVRVENGELIIEAHQEPISDEFGSYEYTSGKVLTEGLFSQTYGRFEARMRLPAGQGFWPAFWMMPENDQYGGWAASGEIDIMENAGGTPYKVGGAIHYGGPWPENQFQAGDYFFPDGTDATGYHEYAVEWEPGEIRWYVDGNLYQTINDWYSTGGAYPAPFDQDFHLILNLAVGGWYGGNPDGSTPFPSTMAVDYVRVYER

SEQ ID NO: 15 - protein, 260 aa, Bacillus sp.
TETEEGISIEQEGWNLVWNDEFDGDSLDQSKWRYDIGNGQPNLPGWGNEELQYYNDDPKNVRVENGELIIEAHQEPISDEFGSYEYTSGKVLTEGLFSQTYGRFEARMRLPAGQGFWPAFWMMPENDQYGGWAASGEIDIMENAGGTPYKVGGAIHYGGPWPENQFQAGDYFFPDGTDATGYHEYAVEWEPGEIRWYVDGNLYQTINDWYSTGGAYPAPFDQDFHLILNLAVGGWYGGNPDGSTPFPSTMAVDYVRVYER

SEQ ID NO: 16 - DNA, 840 bp, Bacillus sp.
  sig_peptide 1..69
  mat_peptide 70..837
  source 1..840: mol_type = genomic DNA
  CDS 1..837
atgaaaaagacgattggtacgttatgtgtactcgccttgctggcaaccactcctgttggagccggagctgcgactgcgccagaaccgcaaaacgattggaacctcgtttggagcgacgaatttgatggaaattctcttgattcaagcaaatggcgctacgatattggaaatgggcagccgaatttgcccggttggggaaatgaagagttacaatactactcagatgaccctaaaaatgtgcgagttgaaaacggagaattggtgattgaagcacaccaagaaaccgtatctgatccatacggtacgtatccgtacacatcaggaaaagtcttgactgacgggaagtttagccagacgtacgggcggtttgaagcaagaatgcgcttacctgccggacaagggttttggccagcattttggatgatgccagaaaatgaccgttatggcggctgggcggcttccggagaaatcgatattatggaaaacgctggcgccacgccatataagactggcggtgcgatccactatggcggaccttggccagaaaaccagtttcaagctggtgattactatttcccagactggacaaacgcaaccgattaccatgaatatgcggtcgaatgggagccaggcgaaattcgctggtacgtcgacgggaatttgtatcaaacgattaatgattggtattccgttggaggtagctatcctgcaccgtttgaccaagattttcatttaattcttaacctagctgtcggtgggtggtacggcggcaaccccgacgcgacaacgccattcccgtccactatggccgttgattatgtccgggtgtatcaacggtaa

SEQ ID NO: 17 - protein, 279 aa, Bacillus sp.
MKKTIGTLCVLALLATTPVGAGAATAPEPQNDWNLVWSDEFDGNSLDSSKWRYDIGNGQPNLPGWGNEELQYYSDDPKNVRVENGELVIEAHQETVSDPYGTYPYTSGKVLTDGKFSQTYGRFEARMRLPAGQGFWPAFWMMPENDRYGGWAASGEIDIMENAGATPYKTGGAIHYGGPWPENQFQAGDYYFPDWTNATDYHEYAVEWEPGEIRWYVDGNLYQTINDWYSVGGSYPAPFDQDFHLILNLAVGGWYGGNPDATTPFPSTMAVDYVRVYQR

SEQ ID NO: 18 - protein, 256 aa, Bacillus sp.
ATAPEPQNDWNLVWSDEFDGNSLDSSKWRYDIGNGQPNLPGWGNEELQYYSDDPKNVRVENGELVIEAHQETVSDPYGTYPYTSGKVLTDGKFSQTYGRFEARMRLPAGQGFWPAFWMMPENDRYGGWAASGEIDIMENAGATPYKTGGAIHYGGPWPENQFQAGDYYFPDWTNATDYHEYAVEWEPGEIRWYVDGNLYQTINDWYSVGGSYPAPFDQDFHLILNLAVGGWYGGNPDATTPFPSTMAVDYVRVYQR

SEQ ID NO: 19 - protein, 27 aa, Bacillus clausii
MKKPLGKIVASTALLISVAFSSSIASA

SEQ ID NO: 20 - protein, 8 aa, Synthetic construct
HHHHHHPR

SEQ ID NO: 21 - protein, 488 aa, Synthetic construct
HHHHHHPRADLSQWRLVWSDEFDGPNGSAPDPNKWNLVHAGGGFGNNELQYYTNRRDNSYLENGSLVIKAQKETYNGHAYTSAKLTSQNKGDWKYGRFEIRAKLPYGRSVWPAIWMMPTDSVYGGWPKSGEIDIMENRGDQMNKISGTIHYGNDWPNNTWSGASYNLPGGQSFADDFHTFAIEWEEGIIRWYVDDILYSTKTDWFTPSAPYPAPFDQRFYMQLNVAIGGPNTPFTGFQPPDDSVLPQKMYVDYVRVYERIGSSSSTPIPGKIEAENYSAMNGIQTEQTTDTGGGLNVGWVDAGDWLDYSVNVQTSGTYKVQLRVANALSTGQLQLRSGGTTLATVNVPNTGGWQSWQTIETTVNLTAGQQTLRVYATQRGFNLNWLNFIQDGGSGGGGEHVTADYTAGVSRVSASQAKIYFTPVTPARYVDVHYTVNSGGQLNVRMTNNNGTWETAVNNLKSGDVIRYWFTYEKNGPQYESPEYTYTH

SEQ ID NO: 22 - protein, 270 aa, Synthetic construct
HHHHHHPRAPQQKNGWKLVWNDEFDGKEIDRTKWKHVTGGSGFGNNEDQFYTEDAANSYIEDGKLVIKALKQEHGGKPYTSAKLITEGYADWTYGRFEFRAKMPLGKGMWPAIWMMPTDMEKYGGWPSSGEIDIMEYLGHEPEQVHGTLHMGNPHIYRGGKVSLEDGMFAEAFHDFALEWTPSGMKWYVDDKEFYQTTTWFTRKDEAADNEPFPAPFDRAFFLQLNLAVGGNWPGYPDETTVFPQTFELEYVRVYQPADGNYETANDTAK

SEQ ID NO: 23 - protein, 393 aa, Synthetic construct
HHHHHHPRAYNLVWSDEFNGTSIDSNNWSFEVGTGSGGWGNNELEYYTSRSENARIENGNLVIEARKESYGGMNYTSARLKTQGKKSFQYGRIEARIKMPNGQGLWPAFWTLGSDIGTVGWPKSGEIDIMEHVNNDNNTNGYIHWDANGQADYGGPSGYVDVTQYHVYSIEWTPSAIKWFIDGTQFWEANIANNINSTEEFHKPHFILLNMAVGGNWPGAPNAGTAFPAKMYVDYVRVYQDNGTPQPSNGIVSGGTYKLINVNSGKALDVQSAGTTPGTNVQIWTDNGTGAQKWTIYQNADGSYKLVNVNSALALDVASSGTADGTNVQAWTDNGTGAQKWNIIANGDGSYKLINTNSGKALDVSGSGTADGTNVQIWNDNGTGAQKWNLIKL

SEQ ID NO: 24 - protein, 390 aa, Synthetic construct
HHHHHHPRAPNWNLVWSDEFNGNALNSANWSAEIGTGQNGWGNNELQYYTNRPENVRVADGNLVITARKESYGGMNYTSARIKTQNLKSFTYGKIEARIKLPSGQGLWPAFWMLGSNINAVGWPGCGEIDIMERVNNNPHVNGTVHWDAGGHADYGRVSGNLDFSQYHVYSIEWDSKYIRWFVDGQEYNAFYIENGTGNTEEFQRPFFLLLNLAVGGNWPGSPDPSTPFPAQMLVDYVRVYQASPGIVNGGIYTIASKASGKVMDVVDVSTQSGAKIQQWTNYVANNQKFKVESAGDGYYKLTAVHSGKVLDVPNSSTSSGVQLQQWDDNGSAAQRWSIVDAGGGYYKIVSKTNGLVVDVSGSSTADGATVQQWSDNGTDAQKWSFVKVN

SEQ ID NO: 25 - protein, 268 aa, Synthetic construct
HHHHHHPRTETEEGISIEQEGWNLVWNDEFDGDSLDQSKWRYDIGNGQPNLPGWGNEELQYYNDDPKNVRVENGELIIEAHQEPISDEFGSYEYTSGKVLTEGLFSQTYGRFEARMRLPAGQGFWPAFWMMPENDQYGGWAASGEIDIMENAGGTPYKVGGAIHYGGPWPENQFQAGDYFFPDGTDATGYHEYAVEWEPGEIRWYVDGNLYQTINDWYSTGGAYPAPFDQDFHLILNLAVGGWYGGNPDGSTPFPSTMAVDYVRVYER

SEQ ID NO: 26 - protein, 264 aa, Synthetic construct
HHHHHHPRATAPEPQNDWNLVWSDEFDGNSLDSSKWRYDIGNGQPNLPGWGNEELQYYSDDPKNVRVENGELVIEAHQETVSDPYGTYPYTSGKVLTDGKFSQTYGRFEARMRLPAGQGFWPAFWMMPENDRYGGWAASGEIDIMENAGATPYKTGGAIHYGGPWPENQFQAGDYYFPDWTNATDYHEYAVEWEPGEIRWYVDGNLYQTINDWYSVGGSYPAPFDQDFHLILNLAVGGWYGGNPDATTPFPSTMAVDYVRVYQR

SEQ ID NO: 27 - protein, 272 aa, Thermotoga maritima
MEDEDKVEDWQLVWSQEFDDGVIDPNIWNFEIGNGHAKGIPGWGNGELEYYTDENAFVENGCLVIEARKEQVSDEYGTYDYTSARMTTEGKFEIKYGKIEIRAKLPKGKGIWPALWMLGNNIGEVGWPTCGEIDIMEMLGHDTRTVYGTAHGPGYSGGASIGVAYHLPEGVPDFSEDFHIFSIEWDEDEVEWYVDGQLYHVLSKDELAELGLEWVFDHPFFLILNVAVGGYWPGYPDETTQFPQRMYIDYIRVYKDMNPETITGVEHHHHHH

SEQ ID NO: 28 - protein, 7 aa, Synthetic construct
  VARIANT 3: note = X= any amino acid
  VARIANT 5: note = X= any amino acid
GEXDXME

SEQ ID NO: 29 - protein, 10 aa, Synthetic construct
  VARIANT 2: note = X= any amino acid
  VARIANT 5: note = X= any amino acid
  VARIANT 7..9: note = X= any amino acid
GXGNXEXXXY

SEQ ID NO: 30 - protein, 6 aa, Synthetic construct
  VARIANT 2: note = X= any amino acid
  VARIANT 4: note = X= any amino acid
NXAXGG

SEQ ID NO: 31 - skipped

SEQ ID NO: 32 - protein, 7 aa, Synthetic construct
GEIDIME

SEQ ID NO: 33 - protein, 10 aa, Synthetic construct
  VARIANT 2: note = X= F or W
  VARIANT 5: note = X= F or W
  VARIANT 7: note = X= F or W
  VARIANT 8: note = X= Q or E
  VARIANT 9: note = X= F or W
GXGNXEXXXY

SEQ ID NO: 34 - protein, 10 aa, Synthetic construct
  VARIANT 5: note = X = any amino acid
  VARIANT 7: note = X = any amino acid
  VARIANT 9: note = X = any amino acid
GFGNXEXQXY

SEQ ID NO: 35 - protein, 10 aa, Synthetic construct
  VARIANT 5: note = X = any amino acid
  VARIANT 7: note = X = any amino acid
  VARIANT 9: note = X = any amino acid
GFGNXEXEXY

SEQ ID NO: 36 - protein, 10 aa, Synthetic construct
  VARIANT 5: note = X = any amino acid
  VARIANT 7: note = X = any amino acid
  VARIANT 9: note = X = any amino acid
GWGNXEXQXY

SEQ ID NO: 37 - protein, 10 aa, Synthetic construct
  VARIANT 5: note = 7
  VARIANT 7: note = X = any amino acid
  VARIANT 9: note = X = any amino acid
GWGNXEXEXY

SEQ ID NO: 38 - protein, 5 aa, Synthetic construct
YTSGK

SEQ ID NO: 39 - protein, 5 aa, Synthetic construct
YTSGR

SEQ ID NO: 40 - protein, 5 aa, Synthetic construct
YTSAK

SEQ ID NO: 41 - protein, 5 aa, Synthetic construct
YTSAR

SEQ ID NO: 42 - protein, 7 aa, Synthetic construct
  VARIANT 1: note = X= L or M
  VARIANT 3: note = X = any amino acid
  VARIANT 5: note = X = any amino acid
XNXAXGG

SEQ ID NO: 43 - protein, 7 aa, Synthetic construct
  VARIANT 3: note = X = any amino acid
  VARIANT 5: note = X = any amino acid
LNXAXGG

SEQ ID NO: 44 - protein, 7 aa, Synthetic construct
  REGION 1..7: note = Motif
  SITE 3: note = MISC_FEATURE - Xaa = any amino acid
  SITE 5: note = MISC_FEATURE - Xaa = any amino acid
MNXAXGG

SEQ ID NO: 45 - protein, 482 aa, Synthetic construct
HDGTNGTIMQYFEWNVPNDGQHWNRLHNNAQNLKNAGITAIWIPPAWKGTSQSDTGYGAYDLYDLGEFNQRGTVRTKYGTKAELERAIRSLKANGIQVYGDVVMNHKAGADQTEQVQAVEVNPQNRNQEVSGTYQIEAWTGFNFPGRGNQHSSFKWRWYHFDGTDFDQSRGLSNRIYKFRTKAWDWEVDTEFGNYDYLMYADLDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLAARQQYAYGTQHDYLDNQDVIGWTREGDSAHAGSGLATVMSDGPGGSKTMYVGTAHAGQVFKDITGNRTDTVTINSAGNGTFRCNKGSVSIWVKQ

SEQ ID NO: 46 - protein, 485 aa, Bacillus sp.
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 47 - protein, 483 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRGNTHSNFKWRWYHFDGVDWDQSRKLNNRIYKFRGKGWDWEVDTEFGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQKHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 48 - protein, 483 aa, Bacillus licheniformis [sequence omitted]

SEQ ID NO: 49 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 50 - protein, 481 aa, Synthetic construct
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 51 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 52 - protein, 485 aa, Bacillus sp.
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 53 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 54 - protein, 485 aa, Bacillus sp.; identical to SEQ ID NO: 51

SEQ ID NO: 55 - protein, 485 aa, Bacillus sp.
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 56 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 57 - protein, 484 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 58 - protein, 485 aa, Bacillus sp.; identical to SEQ ID NO: 46

SEQ ID NO: 59 - protein, 483 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 60 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 61 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 62 - protein, 485 aa, Bacillus sp.; identical to SEQ ID NO: 51

SEQ ID NO: 63 - protein, 484 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYTFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 64 - protein, 485 aa, Bacillus halmapalus; identical to SEQ ID NO: 53

SEQ ID NO: 65 - protein, 485 aa, Synthetic construct
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 66 - protein, 485 aa, Bacillus sp.; identical to SEQ ID NO: 60

SEQ ID NO: 67 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 68 - protein, 311 aa, Synthetic construct
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 69 - protein, 270 aa, Synthetic construct
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGAADGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 70 - protein, 269 aa, Synthetic construct
AQSVPWGIRRVQAPTAHNRGLTGSGVKVAVLDTGISTHPDLNIRGGASFVPGEPSTQDGNGHGTHAAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLDGTSMATPHVAGAAALVKQKNPSWSNVRIRNHLKNTATSLGSTNLYGSGLVNAEAATR

SEQ ID NO: 71 - protein, 773 aa, Bacillus sp.
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 72 - protein, 435 aa, Humicola insolens
MARGTALLGLTALLLGLVNGQKPGETKEVHPQLTTFRCTKRGGCKPATNFIVLDSLSHPIHRAEGLGPGGCGDWGNPPPKDVCPDVESCAKNCIMEGIPDYSQYGVTTNGTSLRLQHILPDGRVPSPRVYLLDKTKRRYEMLHLTGFEFTFDVDATKLPCGMNSALYLSEMHPTGAKSKYNPGGAYYGTGYCDAQCFVTPFINGLGNIEGKGSCCNEMDIWEANSRASHVAPHTCNKKGLYLCEGEECAFEGVCDKNGCGWNNYRVNVTDYYGRGEEFKVNTLKPFTVVTQFLANRRGKLEKIHRFYVQDGKVIESFYTNKEGVPYTNMIDDEFCEATGSRKYMELGATQGMGEALTRGMVLAMSIWWDQGGNMEWLDHGEAGPCAKGEGAPSNIVQVEPFPEVTYTNLRWGEIGSTYQEVQKPKPKPGHGPRSD

SEQ ID NO: 73 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 74 - protein, 299 aa, Thielavia terrestris
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 75 - protein, 524 aa, Paenibacillus polymyxa
VVHGQTAKTITIKVDTFKDRKPISPYIYGTNQDLAGDENMAARRLGGNRMTGYNWENNMSNAGSDWQHSSDNYLCSNGGLTQAECEKPGAVVTSFHDQSLKLGTYSLVTLPMAGYVAADGNGSVQESEAAPSARWNQVVNAKNAPFQLQPDLNDNYVYVDEFVHFLVNKYGTASTKAGVKGYALDNEPALWSHTHPRIHPEKVGAKELVDRSVSLSKAVKAIDAGAEVFGPVLYGFGAYKDLQTAPDWDSVKGNYSWFVDYYLDQMRLSSQVEGKRLLDVFDVHWYPEAMGGGIRITNEVGNDETKKARMQAPRTLWDPTYKEDSWIAQWFSEFLPILPRLKQSVDKYYPGTKLAMTEYSYGGENDISGGIAMTDVLGILGKNDVYMANYWKLKDGVNNYVSAAYKLYRNYDGKNSTFGDTSVSAQTSDIVNSSVHASVTNASDKELHLVVMNKSMDSAFDAQFDLSGAKTYISGKVWGFDKNSSQIKEAAPITQISGNRFTYTVPPLTAYHIVLTTGNDTSPV

SEQ ID NO: 76 - protein, 214 aa, Melanocarpus albomyces
ANGQSTRYWDCCKPSCGWRGKGPVNQPVYSCDANFQRIHDFDAVSGCEGGPAFSCADHSPWAINDNLSYGFAATALSGQTEESWCCACYALTFTSGPVAGKTMVVQSTSTGGDLGSNHFDLNIPGGGVGLFDGCTPQFGGLPGARYGGISSRQECDSFPEPLKPGCQWRFDWFQNADNPSFTFERVQCPEELVARTGCRRHDDGGFAVFKAPSA

SEQ ID NO: 77 - protein, 269 aa, Thermomyces lanuginosus [sequence omitted]

SEQ ID NO: 78 - protein, 298 aa, Bacillus bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVF

SEQ ID NO: 79 - protein, 297 aa, Paenibacillus species
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 80 - protein, 464 aa, Bacillus hemicellulosilyticus
QTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ

SEQ ID NO: 81 - protein, 330 aa, Paenibacillus woosongensis
IRTGTLNNPEATAEARALMNYLLSQYGQKIISGQQTLEDVEWIKQQTGKYPAIFSTDLMDYSPSRVDHGASSTEVEKMIEWYKRGGIVSLCWHWNAPKGIGGNEPGNEWWRGFYTEFTTFDVEYALNHPDSEDYQLLIRDIDAIAVQLKRLQEANVPVLWRPLHEAEGTWFWWGAKGPEPAKQLYRLMYDRLTNDHKLNNLIWVWNSEKKDWYPGDDVVDMVSVDIYNPAGDYNPSIAKYEALVSLADNKKMAALAENGPIPDPDALQEYGADWSFFSTWTGDYIRDGKTNTIEHLKKVYQHDYVITLDELPADCTPILMIRQRMVNQQG

SEQ ID NO: 82 - protein, 498 aa, Paenibacillus woosongensis
MNMEGTPSVSPTNSITVTFANAVLEGYGIEKRGSVKEDDDTLYDGEGYISYFFDEIGGAAEPVGSAAFTVDAAKAGLYELSLGYYIPEGYGDKVTRIQINGEGTGELTLDAPAAGTVRAEKMVSKVLLNAGSNTIQIMRGWGYYGIEHIKLAPANEAPPSNKLNAEDSIRTGTLNNPEATAEARALMNYLLSQYGQKIISGQQTLEDVEWIKQQTGKYPAIFSTDLMDYSPSRVDHGASSTEVEKMIEWYKRGGIVSLCWHWNAPKGIGGNEPGNEWWRGFYTEFTTFDVEYALNHPDSEDYQLLIRDIDAIAVQLKRLQEANVPVLWRPLHEAEGTWFWWGAKGPEPAKQLYRLMYDRLTNDHKLNNLIWVWNSEKKDWYPGDDVVDMVSVDIYNPAGDYNPSIAKYEALVSLADNKKMAALAENGPIPDPDALQEYGADWSFFSTWTGDYIRDGKTNTIEHLKKVYQHDYVITLDELPADCTPILMIRQRMVNQQG

SEQ ID NO: 83 - protein, 312 aa, Paenibacillus illinoisensis
ASPQAKALMKFMTNQYGKKIISGQQTLEDAAWIYQQTGKYPALVSSDLMDYSPSRVENGSTSNEVEKMMEWYKRGGIVSLSWHWNAPKGIGSNEPGHEWWRGFNTEFTTFDVEYALNHPESEDYKLLIRDIDAIATQLKRLQEHHIPVLWRPLHEAEGGWFWWGAKGPEPAKKLYRLMYERLTEKHGLNNLIWVWNSVKEEWYPGDDVVDMVSVDIYNPPGDYSPNIAKYDELLFLSKHKKLVALAENGPIPDPDLLQTYGAHWSYFNTWTGDVLRDGKTNTKEHLKKVYNHDNVITLDELPKGLYDSPRWK

SEQ ID NO: 84 - protein, 491 aa, Paenibacillus illinoisensis
AITVPGFVVEPHTSSDQNQAIIATFKDASIEGYGIKKRDEATAKAEDDLYDGTGYISYFFEEDEKATVQKGSATFQVKAPENGLYELSLGYYIPEGNGDKATSIQVNGSGAGELTLSAPKPGTVRAEKKMTKVLLNSGNNSIQILRGWGYYGIEYIKLERVEPRITTQKTMMDPLSNSKASPQAKALMKFMTNQYGKKIISGQQTLEDAAWIYQQTGKYPALVSSDLMDYSPSRVENGSTSNEVEKMMEWYKRGGIVSLSWHWNAPKGIGSNEPGHEWWRGFNTEFTTFDVEYALNHPESEDYKLLIRDIDAIATQLKRLQEHHIPVLWRPLHEAEGGWFWWGAKGPEPAKKLYRLMYERLTEKHGLNNLIWVWNSVKEEWYPGDDVVDMVSVDIYNPPGDYSPNIAKYDELLFLSKHKKLVALAENGPIPDPDLLQTYGAHWSYFNTWTGDVLRDGKTNTKEHLKKVYNHDNVITLDELPKGLYDSPRWK

SEQ ID NO: 85 - protein, 699 aa, Neobulgaria sp.
TVFEAEKGTLAGGLTIATDVTGYTGTGYVTNFADAAALLTFTVNGLTAGSYDLTLTYSAQYGDKFTTVSVNGASGIEVAITNVTTATWTTATIGTFTLTAGDNTVSCADDWGWYLIDSLTVIPTPAKPITIVDVSNGATAQAEDGILTGTTVGTTTAGYTGTGYVTGFTATGTQVTINLSSTKQALYDVVVRYAAIYGQKYTTMQLNGVGGSEILLLDTTTATSPWANATAGQVLLASGNNTLTFMNDWGWYFIDAVYVTPSPAPAPHKVTNALVDAKALASTHALFNTLLAKYGSGDIFSGQADPTGVTWIESNLGTTKTPAIIGLDMIEYSPTRVLYGSTSTAVEDAIAFDKRGGMVAFQWHWNAPADLINNDTVPWWKGFYSYGTTFNLTAALANPSGSDYALLISDMDAIAVQLLRLQAAGVPVLWRPLHEADGTWFWWGNFGAASCVSLYRIMYDRYTNYHGLHNLIWVWNSVTPSWYPGADVVDILGYDSYPAVGDHGPVSSQYNALITLGGDTKLVTLPEVGNIPDPAILKLYHADWSYFVTWNQDYILTDTYNPLAFKQQVYNDPTVLKLTDLGNWKGAATSTIVSSTSKVSTTTSSLITSTTKKTSSSTVVSTTSSTVKTTSTTSKVSSSTTKVSSTTKVTTTSTTTSAVATATAGHWGQCGGTGWTGPTVCASGFTCTAVSPPYYYQCL

SEQ ID NO: 86 - protein, 452 aa, Preussia aemulans
QTVIYQAEQAKLSGVTVEFSIIKQVVGTGYVEGFDESTDSITFTVESTTAALYDLALTYNGPYGDKYTNVVLNNAAGSQVSLPATTAWTTVPAGQVLLNAGANTIQIQNNWGWYLVDSISLKPAATRGAHQITTKPVNKNANSDAKALLKYLGSIYGKKILSGQQDLSSLDWVTKNVGKTPAVLGLDTMDYSESRKSRGAVSTDVDKAIAFAKKGGIVTFCWHWGAPTGLFDSAAQPWYRGFYTDATDFNIETALKDTTNANYTLLMKDIDTIAVQLKKLQDAGVPVIWRPLHEAEGGWFWWGAKGPEPAKKLWKIMYDRLTNQHGLNNLVWTWNSVAPNWYPGDDTVDIVSADTYSQGDHGPISATYNNLLALTNDTKIIAAAEIGSVMEPAQLQAYQADWVYFCVWSGEFIDGGVWNSLDFLKKVYNDPYVLTLDEIQGWKTARGKPRVS

SEQ ID NO: 87 - protein, 312 aa, Yunnania penicillate
APSTTPVNEKATDAAKNLLSYLVEQAANGVTLSGQQDLESAQWVSDNVGKWPAILGIDFMDYSPSRVEYGAVGSTVPDAISYDSDGGIVTFCWHWGSPSGTYNTTDQPWWSNFYTEATAFDIAAAMDDPDSADYNLLVRDIDAISELLLQLQDLDIPILWRPLHEAEGGWFWWGAKGPEACIALYRLMFDRMTNHHGLNNLLWVWNSVDPSWYPGNDVVDIVSADIYADAGDHSPQEETFASLQSLTGDTKLVALGEVGNIPDPASTGGVADWAYWVTWNGDFIKGEDYNPLEYKKEVFSAENIITRDEVDV

SEQ ID NO: 88 - protein, 327 aa, Myrothecium roridum
GTIENRQWLTYNPVDSAATTEARALLRYIQSQYGWRYLSGQQERAEVQWLKSNIGKTPAIQGSDLIDYSPSRVSYGATSTAVEDAIAFDRQGGIVTFTWHWNAPNCLYNSADQPWYFGFYTKATCFNIQAALAQGSNGADYKLLIRDIDAIAVQLKRLRDAKVPILFRPLHEPDGAWFWWGAKGSGPFKQLWDILYDRLTKYHGLHNMLWVCNTEKSDWYPGNNKCDIATTDVYVNAGDHSVQKSHWDALYGVSGGQRILALGEVGVIPDPERQASENVPWAYWMTWNGYFIRDGNYNSRNFLQSTFSNARVVTLDGTSPLGNWKSS

SEQ ID NO: 89 - protein, 452 aa, Chaetomium brasiliense
VPCGGGSNSGPRTYEAEDADLTGTNIDTAQSGFTGSGYVTGFDQATDKVTFKVDSPSLKLYDLSIRVAAIYGEKRTNVVLNNGASSEVYFAASETFTTVAAGQVLLNEGANTIDLVSNWGWYLIDSITLTPSTQRPPHNINPSPVNPSANADAKGLYTYLRSIYGKKILSGQQELSWSNWITTQTGKTPALVSVDLMDYSPSRVERGTVGTAVEEAITHAQRGGIVSVLWHWNAPTGLYDTEENKWWSGFYTRATDFDVAAALSSTTNANYTLILRDIDAIAVQLKKLQDAGVPVLFRPLHEAEGGWFWWGAKGAEPCKKLYALLYDRLTNYHKINNLIWVWNSILEEWYPGDATVDILSADVYAQGNGPISTQYNQLIELGKDKKMIAAAEVGAAPLPNLLQAYEAHWLWFTVWGDTFINNAEWNSVDVLKQVYTSDYVLTLDEIQGWRGA

SEQ ID NO: 90 - protein, 541 aa, Ascobolus stictoideus
QTYTLEAEAGTLTGVTVMNEIAGFSGTGYVGGWDEDADTVSLTFTSDATKLYDVKIRYSGPYGSKYTRISYNGATGGDISLPETTEWATVNAGQALLNAGSNTIKLHNNWGWYLIDAVILTPSVPRPPHQVTDALVNTNSNAVTKQLMKFLVSKYHKAYITGQQELHAHQWVEKNVGKSPAILGLDFMDYSPSRVEFGTTSQAVEQAIDFDKRGGIVTFAWHWNAPSGLINTPGSEWWRGFYTEHTTFDVAAALQNTTNANYNLLIRDIDAIAVQLKRLQTAGVPVLWRPLHEAEGGWFWWGAKGPEPAKKLYKILYDRLTNYHKLNNLIWVWNSVAKDWYPGDEIVDVLSFDSYPAQPGDHGPVSAQYNALVELGKDKKLIAATEVGTIPDPDLMQLYESYWSFFVTWEGEFIENGVHNSLEFLKKLYNNSFVLNLDTIQGWKNGAGSSTTTVKSTTTTPTTTIKSTTTTPVTTPTTVKTTTTPTTTATTVKSTTTTAGPTPTAVAGRWQQCGGIGFTGPTTCEAGTTCNVLNPYYSQCL

SEQ ID NO: 91 - protein, 526 aa, Chaetomium virescens
PRDPGATARTFEAEDATLAGTNVDTALSGFTGTGYVTGFDQAADKVTFTVDSASTELYDLSIRVAAIYGDKRTSVVLNGGASSEVYFPAGETWTNVAAGQLLLNQGSNTIDIVSNWGWYLIDSITLTPSTPRPAHQINEAPVNAAADKNAKALYSYLRSIYGKKILSGQQELSLSNWIAQQTGKTPALVSVDLMDYSPSRVERGTVGTAVEEAIQHHNRGGIVSVLWHWNAPTGLYDTEEHRWWSGFYTSATDFDVAAALSSTTNANYTLLIRDIDAIAVQLKRLQSAGVPVLFRPLHEAEGGWFWWGAKGPEPAKKLWGILYDRVTNHHQINNLLWVWNSILPEWYPGDATVDILSADVYAQGNGPMSTQYNQLIELGKDKKMIAAAEVGAAPLPDLLQAYEAHWLWFTVWGDSFINNADWNSLDTLKKVYTSDYVLTLDEIQGWQGSTPSATTTSSTTTPSATTTTTTPSTTATTATPSATTTASPVTYAEHWGQCAGKGWTGPTTCRPPYTCKYQNDWYSQCL

SEQ ID NO: 92 - protein, 399 aa, Bacillus subtilis [sequence omitted]

SEQ ID NO: 93 - protein, 182 aa, Bacillus cibi [sequence omitted]

SEQ ID NO: 94 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK